(19) United States
(12) Patent Application Publication
(10) Pub. No.: US 2025/0276086 A1
(43) Pub. Date: Sep. 4, 2025

(54) LIPID NANOPARTICLES FOR TARGETED DELIVERY OF MRNA

(71) Applicants: Trustees of Tufts College, Medford, MA (US); The Brigham and Women's Hospital, Inc., Boston, MA (US)

(72) Inventors: Qiaobing Xu, Lexington, MA (US); Min Qiu, Medford, MA (US); Yan Tang, Boston, MA (US)

(21) Appl. No.: 18/272,024

(22) PCT Filed: Jan. 18, 2022
(86) PCT No.: PCT/US2022/012786

§ 371 Date: Jul. 12, 2023

Related U.S. Application Data
(60) Provisional application No. 63/256,811, filed on Oct. 18, 2021.
(60) Provisional application No. 63/251,791, filed on Oct. 4, 2021.
(60) Provisional application No. 63/138,047, filed on Jan. 15, 2021.

(51) Int. Cl. A61K 47/69

(52) U.S. Cl. CPC A61K 47/6911

(57) ABSTRACT

Disclosed are compositions including a pharmaceutical agent assembled with a lipid composition, wherein the lipid composition comprises a lipidoid having structural Formula (I):

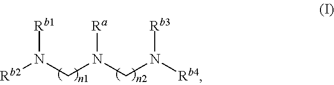

or a pharmaceutically acceptable salt thereof, wherein R^{a}, R^{b1}, R^{b2}, R^{b3}, R^{b4}, n1 and n2 are as described herein.

### RELATED APPLICATIONS

This application is the § 371 National Stage of PCT/US2022/012786, filed Jan. 18, 2022; which claims the benefit of priority to U.S. Provisional Application Nos. 63/138,047, filed Jan. 15, 2021; 63/256,811, filed Oct. 18, 2021; and 63/251,791, filed Oct. 4, 2021.

### GOVERNMENT SUPPORT

This invention was made with government support under grant number UG3 TR002636-01 awarded by the National Institutes of Health. The government has certain rights in the invention.

### BACKGROUND

Genome editing technologies enable the permanent repair of disease-causing genetic mutations. However, the application of this technology has been limited by the technical challenge of achieving safe, effective, and specific in vivo delivery of the CRISPR-Cas9 genome editing components.

### SUMMARY

Disclosed herein is the development of a newly identified lipid nanoparticle (LNP) for specific delivery of CRISPR-Cas9 mRNA to the lung or liver. While LNPs have recently been FDA-approved for delivery of siRNA to the liver, described herein is their application for genome editing. When compared head-to-head, the disclosed delivery platform significantly outperforms the FDA-approved LNP in the efficient delivery of Cas9 mRNA for knockdown of the Angptl3 gene and subsequent regulation of hypercholesterolemia, while matching the safety and specificity of the approved platform.

### BRIEF DESCRIPTION OF THE DRAWINGS

FIG. 1 is a schematic illustration of non-viral lipid nanoparticle (LNP)-mediated in vivo CRISPR-Cas9-based genome editing to induce loss-of-function mutations in Angptl3 to lower blood lipid levels. The Cas9 mRNA and Angptl3-specific single guide RNA (sgAngptl3) are encapsulated in the LNP and delivered to the liver hepatocytes where they cleave the Angptl3 target locus, leading to reduced ANGPTL3 protein. Reduced ANGPTL3 level leads to a dis-inhibition of Lipoprotein lipase (LPL), which allows LPL to regulate the levels of therapeutically-relevant circulating lipids such as low density lipoprotein cholesterol (LDL-C) and triglyceride (TG).

FIGS. 2A-2C show exemplary lipidoid nanoparticles synthesis. FIG. 2A shows lipidoid nanoparticles synthesis with the chemical structure of tail-branched bioreducible lipidoids.

FIG. 2B shows a whole body luciferase bioluminescence intensity of bioreducible LNPs versus MC3 LNP measured in Balb/c mice (n=3) at 6 h post-administration at a firefly luciferase (fLuc) mRNA dose of 0.5 mg/kg. Formulation: lipid/cholesterol/DSPC/DMG-PEG=50/38.5/10/1.5 (molar ratio), lipid/mRNA=12.5/1 (weight ratio).

FIG. 2C is a size and distribution of 306-O12B LNPs formulated with fLuc mRNA measured by DLS.

FIGS. 3A-3F shows the optimization of fLuc mRNA 306-O12B LNP formulations. FIG. 3A shows chemical structures of three different phospholipids.

FIG. 3B shows biodistribution of three different phospholipids of FIG. 3A.

FIG. 3C shows biodistribution of fLuc mRNA LNPs formulated with Cholesterol, DMG-PEG, and different phospholipids (DSPC or DOPE or DOPC). From top-to-bottom, luminescence signal is shown from the heart, liver, spleen, lungs, and kidneys of representative mice. *P<0.05, **P<0.01, ***P<0.001.

FIG. 3D shows formulation parameters of LNPs formulated with cholesterol, DOPC, and DMG-PEG at 7 different mole ratios.

FIG. 3E shows whole body luciferase bioluminescence intensity 6 h post-injection of 0.5 mg/kg fLuc mRNA for LNPs formulated with cholesterol, DOPC, and DMG-PEG at a molar ratio of 50/38.5/10/1.5 with differing 306-O12B/mRNA weight ratios. (n=3).

FIG. 3F shows whole body luciferase bioluminescence intensity of different formulations in Balb/c mice at 6 h post-injection at a fLuc mRNA dose of 0.5 mg/kg

FIGS. 4A & 4B show that 306-O12B LNP-mediated significant levels of in vivo genome editing of Angptl3 in wild-type C57BL/6 mice. FIG. 4A shows 306-O12B LNP-mediated indels percentage and serum ANGPTL3 levels of in vivo genome editing of Angptl3 in wild-type C57BL/6 mice.

FIG. 4B shows 306-O12B LNP-mediated indels percentage following injections of 306-O12B LNP formulated with Cas9 mRNA and sgAngptl3 at a mass ratio of 2:1, 1:1.2 and 1:2. (n=5). 306-O12B LNP was formulated at a molar ratio of [306-O12B:Cholesterol:DSPC:DMG-PEG] of [50:38.5:10:1.5] with a 7.5/1 weight ratio of 306-O12B/total RNAs.

FIGS. 5A-5C show that 306-O12B LNP is more efficient than MC-3 LNP in inducing loss-of-function mutations in Angptl3 through CRISPR-Cas9-based genome editing. FIG. 5A shows next generation sequence analysis of the indels in liver and serum analyses of ANGPTL3 protein, triglyceride, and LDL-C level of mice at day 7 post administrated with Cas9 mRNA and sgAngptl3 coloaded 306-O12B LNP at a total RNA dose of 3.0 mg/kg. MC-3 LNP were used as a positive control. (n=5 or 6). *P<0.05, **P<0.01, ***P<0.001.

FIG. 5B shows editing frequencies of specific edited alleles in each treatment group.

FIG. 5C shows editing frequencies at 9 top predicted off-target sites

FIGS. 6A & 6B show that 306-O12B LNP-mediated CRISPR editing remains durable after one hundred days. FIG. 6A shows next generation sequence analysis of the indels in liver and serum analyses of ANGPTL3 protein, triglyceride, and LDL-C level of mice at day 100 post administrated with Cas9 mRNA and sgAngptl3 coloaded 306-O12B LNP.

FIG. 6B shows serum levels of AST and ALT and TNF-alpha measured at day 100 post-injection. Female C57BL/6 mice were systemically injected with 306-O12B LNP co-formulated with Cas9 mRNA and sgAngptl3 with a single dose at 1.0, 2.0, and 3.0 mg/kg of total RNA. Mice serum were collected at day 100 after injection. (n=5) *P<0.05, **P<0.01, ***P<0.001.

FIG. 7A is a representative TEM images of 306-O12B LNP before formulated with fLuc mRNA. (Scale bar: 500 nm).

FIG. 7B is a representative TEM images of 306-O12B LNP after formulated with fLuc mRNA. (Scale bar: 500 nm).

FIG. 8 shows in vivo imaging of mice injected with fLuc mRNA loaded 306-O12B, 113-O12B, 306-O10B LNP and MC-3 LNP. Images were taken by IVIS imaging system at 6 h post-injection.

FIG. 9 shows encapsulation efficiency of 306-O12B LNP to fLuc mRNA.

FIGS. 10A & 10B show 306-O12B LNP enabled sgLoxP-mediated genome editing in Ai14/Cas9 crossing mice. FIG. 10A is an ex vivo image of organs collected from Ai14/Cas9 mice administrated with LNPs encapsulating sgLoxP at a dose of 0.75 mg/kg, tdTomato fluorescence was detected by IVIS imaging system.

FIG. 10B is a representative immunofluorescence images of liver section showed that tdTomato signal was mainly expressed in liver hepatocytes. (Scale bar: 20 μm).

FIGS. 11A-11C show 306-O12B LNP enabled Cas9/sgLoxP-mediated genome editing in Ai14 mice. FIG. 11A shows a Schematic illustration of codelivery of Cas9 mRNA and LoxP-targeted single guide RNA (sgLoxP) to genetically engineered tdTomato reporter Ai14 mice.

FIG. 11B shows an ex vivo images and a quantification of the ROI of organs collected from Ai14 mice administrated with LNPs encapsulating Cas9 mRNA/sgLoxP (1/1.2, wt) at a total RNA dose of 1.65 mg/kg, tdTomato fluorescence was detected by IVIS imaging system.

FIG. 11C shows confocal fluorescence microscopy of liver section showed that tdTomato signal was mainly expressed in liver hepatocytes. (Scale bar: 20 μm).

FIG. 12 shows size distribution of Cas9 mRNA/sgAngptl3 co-loaded 306-O12B LNP.

FIG. 13 show T7E1 assays performed with genomic DNA from liver samples from mice taken 7 days after receiving the Cas9 mRNA/sgAngptl3 coloaded 306-O12B LNP and MC-3 LNP. Arrows show the cleavage products resulting from the T7E1 assays.

FIG. 14 shows serum levels of AST, ALT and TNF-alpha measured at day 2 post-injection. (n=5).

FIG. 15 shows serum levels of IFN-α, IL-6, and IP-10 in mice. Mice were treated with 306-O12B LNP co-formulated with Cas9 mRNA and sgAngptl3 at 3.0 mg/kg of total RNAs (n=5).

FIG. 16 shows 306-O12B LNP induced long-term editing of Angptl3 after a single administration. C57BL/6 mice were administered 306-O12B LNP co-formulated with Cas9 mRNA and sgAngptl3 at 3 mg/kg. (n=5 for 7 and 100-day time point, n=3 for 150-day time point).

FIG. 17 shows representative liver targeting lipids.

FIGS. 18A-18D show the effects of injection of various lipids. They show ex vivo—8 h—postinjection.

FIGS. 19A-19E show the analysis of lung cell types transfected using Ai14 Cre/lox reporter mice. Formulation: Lipid/Chol/DOPC/DMG-PEG=50/38.5/10/1.5 (molar ratio), Lipid/mRNA=12.5/1 (weight ratio), Dosage: 0.5 mg/kg (10 ug/mouse) (unmodified fLuc-mRNA).

FIG. 19D shows luciferase protein/total protein in the lung after 6 h injection.

FIG. 19E shows analysis of lung cell types transfected using Ai14 Cre/lox reporter mice.

FIG. 20 shows a schematic for in vivo Cre-mRNA delivery—306-N16B, Cre-mRNA dosage: 7.5 ug/mouse/injection, with 2 injections in total.

FIGS. 21A-21C illustrate that the in vivo selectivity of mRNA encapsulated LNPs is governed by the chemical structure of active lipidoids. FIG. 21A is a schematic illustration of fine-tuning of the in vivo organ targeting behavior of mRNA loaded LNPs by simply change the linker group in the lipidoid tails.

FIG. 21B shows the synthetic route and representative chemical structure of lipidoids.

FIG. 21C shows a representative whole-body bioluminescence images of mice taken by the IVIS imaging system. Mice were injected with either Luc mRNA-loaded 306-O12B LNP or 306-N12B LNP at a single dose of 0.5 mg/kg. Pictures were taken at 6 h post-injection. (n=3).

FIGS. 22A and 22B shows a second-generation library of N-series LNPs confirmed linker-governed lung selectivity. The second-generation N-series LNPs were generated by reacting the N16B tail with the amines shown in FIG. 22A.

FIG. 22B shows IVIS images showed that the N-series LNPs mediated specific delivery of fLuc mRNA to the lungs.

FIGS. 23A-23D illustrate that 306-N16B LNP enables specific delivery of Cre mRNA to the lung endothelial cells. FIG. 23A shows schematic illustration of the delivery of Cre mRNA to the lung to activate tdTomato expression via Cre-mediated genetic deletion of the stop cassette in tdTomato transgenic Ai14 mice.

FIG. 23B shows representative ex vivo image of tdTomato fluorescence in edited Ai14 mouse organs captured by using the IVIS imaging system. Mice were i.v. injected with Cre mRNA-loaded 306-N16B LNP at a single dose of 0.75 mg mRNA equiv./kg.

FIG. 23C shows representative immunofluorescence images of lung tissue taken by confocal microscopy. Endothelial cells were stained by FITC-CD31 antibody, epithelial cells were stained by CD326-APC antibody, and macrophages were stained by F4/80-eFluor 660 antibody. Bar: 20 μm.

FIG. 23D shows quantification of the percentage of tdTomato+ cells within defined cell types of the lung by FACS. (n=3).

FIGS. 24A-24E show bioinformatic classification of corona components. FIG. 24A shows quantification of % of total proteins of the top-3 protein components in the protein corona of 306-O12B LNP (n=3).

FIG. 24B shows quantification of % of total proteins of the top-3 protein components in the protein corona of 306-N16B LNP (n=3).

FIG. 24C shows the top 20 most abundant corona proteins categorized based on their calculated molecular weight.

FIG. 24D shows the top 20 most abundant corona proteins were categorized based on their isoelectric point,

FIG. 24E shows the top 20 most abundant corona proteins were categorized based on their and biological function.

FIG. 25A shows distribution of luciferase protein expression in the mice treated with 306-O12B and 306-N16 LNPs. ex vivo image of mouse organs and quantification of luciferase protein in different organs 6 h after administration of mRNA-encapsulated 306-O12B LNP.

FIG. 25B shows distribution of luciferase protein expression in the mice treated with 306-O12B and 306-N16 LNPs. ex vivo image of mouse organs and quantification of luciferase protein in different organs 6 h after administration of mRNA-encapsulated 306-N16B LNP.

FIG. 26 shows a western blot analysis of Cas9 protein expression in different organs in Babl/c mouse following the injection of 306-N16B LNP co-formulated with Cas9 mRNA. Dosage: 0.5 mg equiv. Cas 9 mRNA/kg. Organs were collected at 6 h after injection

FIG. 27 is a Venn diagram showing common and unique proteins absorbed on 306-O12B LNP (light green) and 306-N16B LNP (light blue).

FIGS. 28A-28C show the synthesis and in vivo screening of N-series LNPs.

FIG. 28A shows synthetic route and representative chemical structure of lipidoids. Representative whole-body bioluminescence images of mice.

FIG. 28B and FIG. 28C show in vivo mRNA delivery efficacy of N-series LNPs measured by the IVIS imaging system. Mice were injected with either Luc mRNA-loaded N-series LNPs at a single dose of 0.5 mg/kg. Images were taken at 6 h post-injection. (n=3).

FIGS. 29A-29C show that different pulmonary cell types can be targeted by tuning the head structure of N-series LNPs.

FIG. 29A shows schematic illustration of the delivery of Cre mRNA to the lung to activate tdTomato expression via Cre-mediated genetic deletion of the stop cassette in tdTomato transgenic Ai14 mice.

FIG. 29B shows chemical structure, representative ex vivo image of tdTomato fluorescence in edited Ai14 mouse organs captured by using the IVIS imaging system, representative immunofluorescence images of lung tissue taken by confocal microscopy, and quantification of the percentage of tdTomato+ cells within defined cell types of the lungs by FACS of 306-N16B LNP. Mice were i.v. injected with Cre mRNA-loaded LNPs at a single dose of 0.75 mg mRNA equiv./kg. Endothelial cells were stained by FITC-CD31 antibody, epithelial cells were stained by CD326-PE-Cy7 antibody, and macrophages were stained by F4/80-eFluor 660 antibody. Bar: 20 μm. Quantification of the percentage of tdTomato+ cells within defined cell types of the lung by FACS. (n=3).

FIG. 29C shows chemical structure, representative ex vivo image of tdTomato fluorescence in edited Ai14 mouse organs captured by using the IVIS imaging system, representative immunofluorescence images of lung tissue taken by confocal microscopy, and quantification of the percentage of tdTomato+ cells within defined cell types of the lungs by 113-N16B LNP. Mice were i.v. injected with Cre mRNA-loaded LNPs at a single dose of 0.75 mg mRNA equiv./kg. Endothelial cells were stained by FITC-CD31 antibody, epithelial cells were stained by CD326-PE-Cy7 antibody, and macrophages were stained by F4/80-eFluor 660 antibody. Bar: 20 μm. Quantification of the percentage of tdTomato+ cells within defined cell types of the lung by FACS. (n=3).

FIGS. 30A-30B show proteomics study of protein coronas formed on LNPs.

FIG. 30A is a schematic illustration of different organ targetability of O- and N-series LNPs.

FIG. 30B shows a schematic illustration of interaction of LNPs with proteins in the blood vessel.

FIG. 31A is a schematic illustration of codelivery of Cas9 mRNA and sgLoxP to activate the tdTomato expression in Ai14 mice.

FIG. 31B shows a representative ex vivo image of organs collected from Ca9 mRNA and sgLoxP coloaded 306-N16B LNP treated transgenic Ai14 mice. Mice were injected via tail vein at a dosage of 1.67 mg/kg of total RNA.

FIG. 31C shows a representative microscopy images of the lung and liver dissected from Cas9 mRNA and sgLoxP co-encapsulated 306-N16B LNP treated Ai14 mice. Bar: 100 μm

FIGS. 32A-32C show that hybrid LNP (hLNP) enables specific delivery of mRNA to the TTJ tumor cells in vivo.

FIG. 32A is a schematic illustration of the preparation of hybrid LNP. hLNP was formulated at a molar ratio of 25:25:38.5:10:1.5 of 306-N16B: 306-O12B:Cholesterol:DOPC:DMG-PEG2000.

FIG. 32B shows representative images of immunohistochemistry (IHC) analysis of EGFP in mouse LAM lungs. Syngeneic C57BL/6J mice were tail vein injected with TSC2-deficient TTJ cells to form tumor nodules in the lungs. Mice were tail-vein injected with hLNP loading with GFP mRNA. Lungs were collected after 6 hours of injection.

FIG. 32C is a multiplex immunofluorescence staining showing co-localization of pS6 (a marker of tumor cell) and EGFP. (left) staining of pS6; (right) staining of EGFP.

FIGS. 33A-33E show the therapeutic effect of TSC2 mRNA loaded hLNP in antitumor growth.

FIG. 33A shows a treatment design. Syngeneic C57BL/6J mice were tail vein injected with 2×10⁶ TSC2-deficient TTJ cells to form tumor nodules in the lung. On day 24 after tumor cell inoculation, mice were randomly assigned to 3 groups: untreated control, empty LNPs treatment, and LNPs/mRNA-TSC2 (0.75 mg/kg of mRNA per injection) treatment.

Mice were treated every other day for a total of 5 times. (n=2).

FIG. 33B shows H&E images of lungs dissected from mice receiving different treatments.

FIG. 33C shows fraction of tumor nodules per lung in the 3 treatment groups.

FIG. 33D shows representative images of IHC assessment of ki67, cleaved caspase 3, macrophages, and CD3 in empty hLNP, and TSC2 mRNA loaded hLNP.

FIG. 33E shows quantitative analysis of FIG. 33D. Ten images were analyzed for each group. T cell: CD3 IHC; Apoptosis: cleaved caspase 3 IHC; Proliferation: Ki67 IHC; Macrophage: F4/80 IHC.

FIG. 34A shows in vitro transfection efficiency of LNPs in TTJ cells. Percentage of EGFP positive TTJ cells measured by flow cytometry.

FIG. 34B shows representative fluorescence EGFP images of transfected cells.

FIG. 35A shows in vitro transcription of full-length mouse Tsc2 mRNA assessed by bioanalyzer, showing integrity and full-length of mRNA.

FIG. 35B shows immunoblotting shows suppression of phospho-S6 by wildtype Tsc2 mRNA re-expression in mouse derived TSC2-null TTJ cells.

FIG. 35C shows in vitro transcription of full length human TSC2 mRNA assessed by bioanalyzer.

FIG. 35D shows that immunoblotting shows suppression of phospho-S6 by wildtype TSC2 mRNA re-expression in patient-derived TSC2-deficient cells. 621-101: patient-derived TSC2-deficient cell; 621-103: TSC2-reexpressed in 621-101 cell; last 2 columns: 2 replicates of TSC2 mRNA treatment on 621-101 cells for 24 hours

FIG. 36 show representative images of the lungs dissected from mice receiving various treatments (n=2 mice in each group).

### DETAILED DESCRIPTION

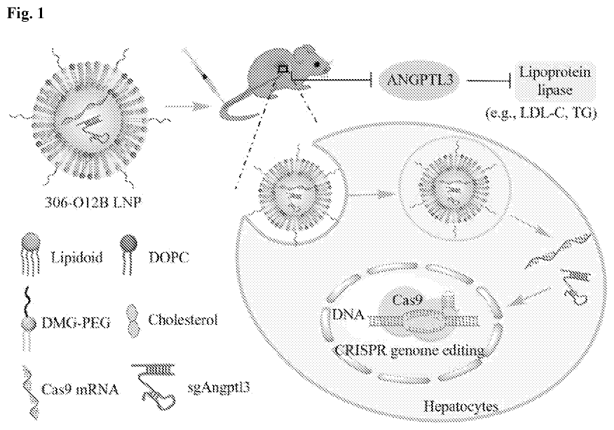

In one aspect, disclosed is a highly potent new non-viral LNP-mediated CRISPR-Cas9 delivery system for the liver delivery of Cas9 mRNA, and demonstrate its efficacy by targeting the Angptl3 gene. The system is composed of a leading tail-branched bioreducible lipidoid (306-O12B) co-formulated with an optimized mixture of excipient lipid molecules, and it successfully co-delivers SpCas9 mRNA and a single guide RNA targeting Angptl3 (sgAngptl3) via at least a single administration (FIG. 1).

Using a library screening approach, it was identified that N-series LNPs (containing an amide bond in the tail) are capable of selectively delivering mRNA to the mouse lung, in contrast to 0-series LNPs (containing an ester bond in the tail) tend to deliver mRNA to the liver. The protein corona was analyzed on the liver- and lung-targeted LNPs using LC/MS and identified a group of unique plasma proteins specifically absorbed onto the surface that may contribute to the targetability of these LNPs. The lipidoid tail structure has a profound impact on the interaction of LNPs with serum proteins and substantially governs the organ-selectivity of LNPs. Different pulmonary cell types can be targeted by simply tuning the headgroup structure of N-series LNPs. The first success of LNP-based RNA therapy is demonstrated herein in a preclinical model of Lymphangioleiomyomatosis (LAM), a destructive lung disease caused by loss-of-functions mutations in the Tsc2 gene. The lung targeting LNP exhibited highly efficient delivery of the mouse Tuberous Sclerosis Complex 2 (Tsc2) mRNA for the restoration of TSC2 tumor suppressor in tumor, and achieved remarkable therapeutic effect in reducing tumor burden. Overall, this study provides new insights into the rational and predictable design and engineering of next-generation organ- and cell-targeted LNPs for mRNA-based therapy.

Safe and efficacious systemic delivery of messenger RNA (mRNA) to specific organs and cells in vivo remains the major challenge in the development of mRNA-based therapeutics. Targeting of systemically administered lipid nanoparticles (LNPs) co-formulated with mRNA has largely been confined to the liver and spleen. Current LNP development strategies mainly rely on labor- and cost-intensive in vitro and in vivo screening processes, and it is currently challenging to design tissue-specific targeting LNPs in a predictable manner. Using a library screening approach, it is identified that N-series LNPs (containing an amide bond in the tail) are capable of selectively delivering mRNA to the mouse lung, in contrast to 0-series LNPs (containing an ester bond in the tail) tend to deliver mRNA to the liver. The protein corona was analyzed on the liver- and lung-targeted LNPs using LC/MS and identified a group of unique plasma proteins specifically absorbed onto the surface that may contribute to the targetability of these LNPs. The lipidoid tail structure has a profound impact on the interaction of LNPs with serum proteins and substantially governs the organ-selectivity of LNPs. Different pulmonary cell types can be targeted by simply tuning the headgroup structure of N-series LNPs. Importantly, The first success of LNP-based RNA therapy is demonstrated herein in a preclinical model of Lymphangioleiomyomatosis (LAM), a destructive lung disease caused by loss-of-functions mutations in the Tsc2 gene. Disclosed lung targeting LNP exhibited highly efficient delivery of the mouse Tuberous Sclerosis Complex 2 (Tsc2) mRNA for the restoration of TSC2 tumor suppressor in tumor, and achieved remarkable therapeutic effect in reducing tumor burden. Overall, this study provides new insights into the rational and predictable design and engineering of next-generation organ- and cell-targeted LNPs for mRNA-based therapy.

A major roadblock in the development of targeted LNPs is difficulty predicting the in vivo targeting behavior of newly designed LNPs due to the limited understanding of the nano-bio interactions between nanoparticles (NPs) and biological components. The outer surface of NPs can be rapidly covered with a layer of serum proteins, referred to as “protein corona”, which remodels the surface property of NPs and substantially affects the interaction of NPs with organs and cells. The lipidoid amine head structure can impact the delivery efficacy and even the in vivo targetability of mRNA loaded LNPs. Imidazole-based synthetic lipidoids preferentially target mRNA to the spleen (X. Zhao et al., Imidazole-Based Synthetic Lipidoids for In Vivo mRNA Delivery into Primary T Lymphocytes. Angew Chem Int Ed Engl 59, 20083-20089 (2020). For the lipidoid tail chemistry, although considerable progress has been made in the understanding of lipidoid tail length, degree of unsaturation, and degree of branching on the effect of mRNA delivery potency, the influence of lipidoid tail structures on in vivo selectivity of LNPs remains poorly understood.

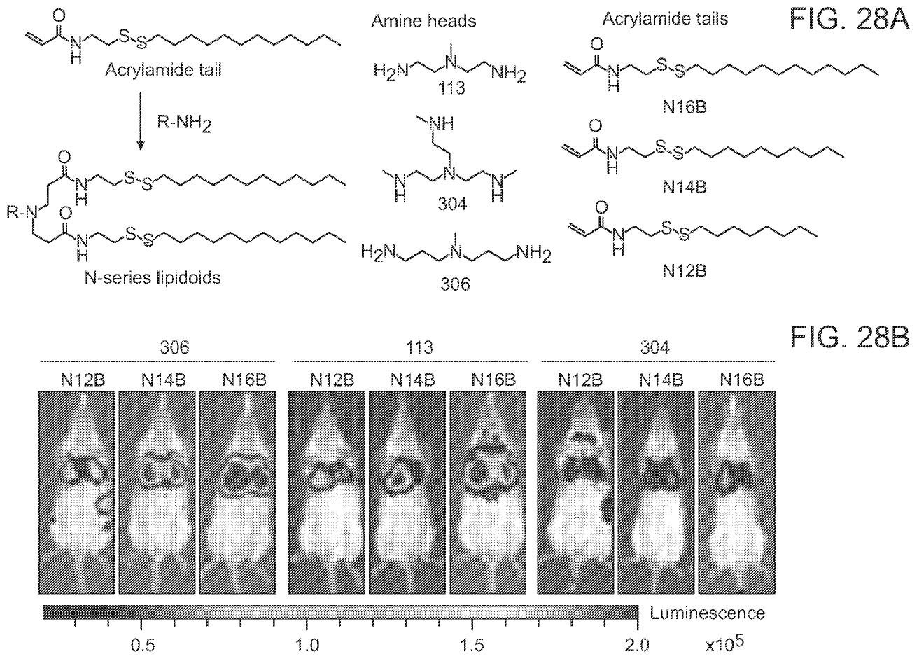

To address this important knowledge gap, a library of amide bond-containing lipidoids (N-series LNPs) was synthesized via Michael-Addition reaction between amine heads and acrylamide tails (FIG. 28A). Surprisingly, from in vivo screening, it was found that the N-series LNPs almost exclusively deliver mRNA to the lung following systemic administration (FIGS. 28B & 28C). Previous study demonstrated that the O-series lipidoids which contain an ester bond in the tails tend to deliver mRNA into the liver (M. Qiu et al., Lipid nanoparticle-mediated codelivery of Cas9 mRNA and single-guide RNA achieves liver-specific in vivo genome editing of Angptl3. Proc. Natl. Acad. Sci. U.S.A. 118, e2020401118 (2021)). To better understand why such a small change induces such striking organ specificity, the underlying mechanisms of these delivery differences was further investigated. Once injected into the bloodstream, the LNPs can selectively govern the adsorption of specific plasma proteins to serve as targeting ligands that direct LNPs to selected organs. Indeed, using proteomics, a group of unique plasma proteins was identified that specifically absorbed on the surface of two representative LNP candidates, 306-O12B and 306-N16B, that may affect the targetability of these LNPs. More importantly, different pulmonary subcellular populations can be targeted by changing the lipidoid head structure of N-series LNPs. Furthermore, the lung-targeting LNPs were evaluated for the in vivo targeted delivery of Tsc2 mRNA to TSC2-deficient cells to restore the expression of the TSC2 tumor suppressor for the treatment of pulmonary Lymphangioleiomyomatosis (LAM), a rare genetic disorder caused by biallelic mutations and loss of function of TSC complex genes. This study provides proof-of-concept that tuning the in vivo organ-targeting behavior of LNPs can be achieved by tailoring the composition of protein corona via simple chemistry. This work provides a novel strategy for the rational design of highly specific organ- and cell-selective LNPs for mRNA-based therapy

In one aspect, disclosed are

### Pharmaceutical Compositions

The compositions and methods of the present invention may be utilized to treat an individual in need thereof. The pharmaceutical composition described herein may comprise a therapeutic or prophylactic composition, or any combination thereof. In certain embodiments, the lipidoid compositions may be assembled with an antigen, an immune modulator, or any combination thereof. In certain embodiments, the individual is a mammal such as a human, or a non-human mammal. When administered to an animal, such as a human, the composition or the lipidoid composition is preferably administered as a pharmaceutical composition comprising, for example, a lipidoid composition of the invention and a pharmaceutically acceptable carrier. Pharmaceutically acceptable carriers are well known in the art and include, for example, aqueous solutions such as water or physiologically buffered saline or other solvents or vehicles such as glycols, glycerol, oils such as olive oil, or injectable organic esters. In preferred embodiments, when such pharmaceutical compositions are for human administration, particularly for invasive routes of administration (i.e., routes, such as injection or implantation, that circumvent transport or diffusion through an epithelial barrier), the aqueous solution is pyrogen-free, or substantially pyrogen-free. The excipients can be chosen, for example, to effect delayed release of an agent or to selectively target one or more cells, tissues or organs. The pharmaceutical composition can be in dosage unit form such as tablet, capsule (including sprinkle capsule and gelatin capsule), granule, lyophile for reconstitution, powder, solution, syrup, suppository, injection or the like. The composition can also be present in a transdermal delivery system, e.g., a skin patch. The composition can also be present in a solution suitable for topical administration, such as a lotion, cream, or ointment.

A pharmaceutically acceptable carrier can contain physiologically acceptable agents that act, for example, to stabilize, increase solubility or to increase the absorption of a lipidoid composition such as a lipidoid composition of the invention. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The preparation or pharmaceutical composition can be a self-emulsifying drug delivery system or a self-microemulsifying drug delivery system. The pharmaceutical composition (preparation) also can be a liposome or other polymer matrix, which can have incorporated therein, for example, a lipidoid composition of the invention. Liposomes, for example, which comprise phospholipids or other lipids, are nontoxic, physiologically acceptable and metabolizable carriers that are relatively simple to make and administer.

The phrase “pharmaceutically acceptable” is employed herein to refer to those lipidoid compositions, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

A pharmaceutical composition (preparation) can be administered to a subject by any of a number of routes of administration including, for example, orally (for example, drenches as in aqueous or non-aqueous solutions or suspensions, tablets, capsules (including sprinkle capsules and gelatin capsules), boluses, powders, granules, pastes for application to the tongue); absorption through the oral mucosa (e.g., sublingually); subcutaneously; transdermally (for example as a patch applied to the skin); and topically (for example, as a cream, ointment or spray applied to the skin). The lipidoid composition may also be formulated for inhalation. In certain embodiments, a lipidoid composition may be simply dissolved or suspended in sterile water. Details of appropriate routes of administration and compositions suitable for same can be found in, for example, U.S. Pat. Nos. 6,110,973, 5,763,493, 5,731,000, 5,541,231, 5,427,798, 5,358,970 and 4,172,896, as well as in patents cited therein.

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the lipidoid composition which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an active composition, such as a lipidoid (e.g., nanoparticle) composition as described herein, with the carrier and, optionally, one or more accessory ingredients. In general, the formulations are prepared by uniformly and intimately bringing into association a lipidoid (e.g., nanoparticle) composition as described herein with liquid carriers, or finely divided solid carriers, or both, and then, if necessary, shaping the product.

Formulations of the invention suitable for oral administration may be in the form of capsules (including sprinkle capsules and gelatin capsules), cachets, pills, tablets, lozenges (using a flavored basis, usually sucrose and acacia or tragacanth), lyophile, powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a lipidoid (e.g., nanoparticle) composition as described herein of the present invention as an active ingredient. Lipidoid compositions may also be administered as a bolus, electuary or paste.

To prepare solid dosage forms for oral administration (capsules (including sprinkle capsules and gelatin capsules), tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium lipidoid compositions; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; (10) complexing agents, such as, modified and unmodified cyclodextrins; and (11) coloring agents. In the case of capsules (including sprinkle capsules and gelatin capsules), tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered lipidoid composition moistened with an inert liquid diluent.

The tablets, and other solid dosage forms of the pharmaceutical compositions, such as dragees, capsules (including sprinkle capsules and gelatin capsules), pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in microencapsulated form, if appropriate, with one or more of the above-described excipients.

Liquid dosage forms useful for oral administration include pharmaceutically acceptable emulsions, lyophiles for reconstitution, microemulsions, solutions, suspensions, syrups and elixirs. In addition to the active ingredient, the liquid dosage forms may contain inert diluents commonly used in the art, such as, for example, water or other solvents, cyclodextrins and derivatives thereof, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active lipidoid compositions, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Dosage forms for the topical or transdermal administration include powders, sprays, ointments, pastes, creams, lotions, gels, solutions, patches and inhalants. The active lipidoid composition may be mixed under sterile conditions with a pharmaceutically acceptable carrier, and with any preservatives, buffers, or propellants that may be required.

The ointments, pastes, creams and gels may contain, in addition to an active lipidoid composition, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an active lipidoid composition, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a lipidoid composition of the present invention to the body. Such dosage forms can be made by dissolving or dispersing the active lipidoid composition in the proper medium. Absorption enhancers can also be used to increase the flux of the lipidoid composition across the skin. The rate of such flux can be controlled by either providing a rate controlling membrane or dispersing the lipidoid composition in a polymer matrix or gel.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion. Pharmaceutical compositions suitable for parenteral administration comprise one or more active lipidoid compositions in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsulated matrices of the subject lipidoid compositions in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

For use in the methods of this invention, active lipidoid compositions can be given per se or as a pharmaceutical composition containing, for example, 0.1 to 99.5% (more preferably, 0.5 to 90%) of active ingredient in combination with a pharmaceutically acceptable carrier.

Methods of introduction may also be provided by rechargeable or biodegradable devices. Various slow release polymeric devices have been developed and tested in vivo in recent years for the controlled delivery of drugs, including proteinaceous biopharmaceuticals. A variety of biocompatible polymers (including hydrogels), including both biodegradable and non-degradable polymers, can be used to form an implant for the sustained release of a lipidoid composition at a particular target site.

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular lipidoid composition or combination of lipidoid compositions employed, or the ester, salt or amide thereof, the route of administration, the time of administration, the rate of excretion of the particular lipidoid composition(s) being employed, the duration of the treatment, other drugs, lipidoid compositions and/or materials used in combination with the particular lipidoid composition(s) employed, the age, sex, weight, condition, general health and prior medical history of the patient being treated, and like factors well known in the medical arts.

A physician or veterinarian having ordinary skill in the art can readily determine and prescribe the therapeutically effective amount of the pharmaceutical composition required. For example, the physician or veterinarian could start doses of the pharmaceutical composition or lipidoid composition at levels lower than that required in order to achieve the desired therapeutic effect and gradually increase the dosage until the desired effect is achieved. By “therapeutically effective amount” is meant the concentration of a lipidoid composition that is sufficient to elicit the desired therapeutic effect. It is generally understood that the effective amount of the lipidoid composition will vary according to the weight, sex, age, and medical history of the subject. Other factors which influence the effective amount may include, but are not limited to, the severity of the patient's condition, the disorder being treated, the stability of the lipidoid composition, and, if desired, another type of therapeutic agent being administered with the lipidoid composition of the invention. A larger total dose can be delivered by multiple administrations of the agent. Methods to determine efficacy and dosage are known to those skilled in the art (Isselbacher et al. (1996) Harrison's Principles of Internal Medicine 13 ed., 1814-1882, herein incorporated by reference).

In general, a suitable daily dose of an active lipidoid composition used in the compositions and methods of the invention will be that amount of the lipidoid composition that is the lowest dose effective to produce a therapeutic effect. Such an effective dose will generally depend upon the factors described above.

If desired, the effective daily dose of the active lipidoid composition may be administered as one, two, three, four, five, six or more sub-doses administered separately at appropriate intervals throughout the day, optionally, in unit dosage forms. In certain embodiments of the present invention, the active lipidoid composition may be administered two or three times daily. In preferred embodiments, the active lipidoid composition will be administered once daily.

The patient receiving this treatment is any animal in need, including primates, in particular humans; and other mammals such as equines, cattle, swine, sheep, cats, and dogs; poultry; and pets in general.

In certain embodiments, lipidoid compositions of the invention may be used alone or conjointly administered with another type of therapeutic agent.

The present disclosure includes the use of pharmaceutically acceptable salts of lipidoid compositions of the invention in the compositions and methods of the present invention. In certain embodiments, contemplated salts of the invention include, but are not limited to, alkyl, dialkyl, trialkyl or tetra-alkyl ammonium salts. In certain embodiments, contemplated salts of the invention include, but are not limited to, L-arginine, benenthamine, benzathine, betaine, calcium hydroxide, choline, deanol, diethanolamine, diethylamine, 2-(diethylamino)ethanol, ethanolamine, ethylenediamine, N-methylglucamine, hydrabamine, 1H-imidazole, lithium, L-lysine, magnesium, 4-(2-hydroxyethyl)morpholine, piperazine, potassium, 1-(2-hydroxyethyl)pyrrolidine, sodium, triethanolamine, tromethamine, and zinc salts. In certain embodiments, contemplated salts of the invention include, but are not limited to, Na, Ca, K, Mg, Zn or other metal salts. In certain embodiments, contemplated salts of the invention include, but are not limited to, 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acid, 1-ascorbic acid, 1-aspartic acid, benzenesulfonic acid, benzoic acid, (+)-camphoric acid, (+)-camphor-10-sulfonic acid, capric acid (decanoic acid), caproic acid (hexanoic acid), caprylic acid (octanoic acid), carbonic acid, cinnamic acid, citric acid, cyclamic acid, dodecylsulfuric acid, ethane-1,2-disulfonic acid, ethanesulfonic acid, formic acid, fumaric acid, galactaric acid, gentisic acid, d-glucoheptonic acid, d-gluconic acid, d-glucuronic acid, glutamic acid, glutaric acid, glycerophosphoric acid, glycolic acid, hippuric acid, hydrobromic acid, hydrochloric acid, isobutyric acid, lactic acid, lactobionic acid, lauric acid, maleic acid, 1-malic acid, malonic acid, mandelic acid, methanesulfonic acid, naphthalene-1,5-disulfonic acid, naphthalene-2-sulfonic acid, nicotinic acid, nitric acid, oleic acid, oxalic acid, palmitic acid, pamoic acid, phosphoric acid, proprionic acid, 1-pyroglutamic acid, salicylic acid, sebacic acid, stearic acid, succinic acid, sulfuric acid, 1-tartaric acid, thiocyanic acid, p-toluenesulfonic acid, trifluoroacetic acid, and undecylenic acid salts.

The pharmaceutically acceptable acid addition salts can also exist as various solvates, such as with water, methanol, ethanol, dimethylformamide, and the like. Mixtures of such solvates can also be prepared. The source of such solvate can be from the solvent of crystallization, inherent in the solvent of preparation or crystallization, or adventitious to such solvent.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like.

### Definitions

Unless otherwise defined herein, scientific and technical terms used in this application shall have the meanings that are commonly understood by those of ordinary skill in the art. Generally, nomenclature used in connection with, and techniques of, chemistry, cell and tissue culture, molecular biology, cell and cancer biology, neurobiology, neurochemistry, virology, immunology, microbiology, pharmacology, genetics and protein and nucleic acid chemistry, described herein, are those well-known and commonly used in the art.

The methods and techniques of the present disclosure are generally performed, unless otherwise indicated, according to conventional methods well known in the art and as described in various general and more specific references that are cited and discussed throughout this specification. See, e.g. “Principles of Neural Science”, McGraw-Hill Medical, New York, N.Y. (2000); Motulsky, “Intuitive Biostatistics”, Oxford University Press, Inc. (1995); Lodish et al., “Molecular Cell Biology, 4th ed.”, W. H. Freeman & Co., New York (2000); Griffiths et al., “Introduction to Genetic Analysis, 7th ed.”, W. H. Freeman & Co., N.Y. (1999); and Gilbert et al., “Developmental Biology, 6th ed.”, Sinauer Associates, Inc., Sunderland, MA (2000).

Chemistry terms used herein, unless otherwise defined herein, are used according to conventional usage in the art, as exemplified by “The McGraw-Hill Dictionary of Chemical Terms”, Parker S., Ed., McGraw-Hill, San Francisco, C.A. (1985).

All of the above, and any other publications, patents and published patent applications referred to in this application are specifically incorporated by reference herein. In case of conflict, the present specification, including its specific definitions, will control.

As used herein, the terms “optional” or “optionally” mean that the subsequently described event or circumstance may occur or may not occur, and that the description includes instances where the event or circumstance occurs as well as instances in which it does not. For example, “optionally substituted alkyl” refers to the alkyl may be substituted as well as where the alkyl is not substituted.

It is understood that substituents and substitution patterns on the compounds of the present invention can be selected by one of ordinary skilled person in the art to result chemically stable compounds which can be readily synthesized by techniques known in the art, as well as those methods set forth below, from readily available starting materials. If a substituent is itself substituted with more than one group, it is understood that these multiple groups may be on the same carbon or on different carbons, so long as a stable structure results.

As used herein, the term “optionally substituted” refers to the replacement of one to six hydrogen radicals in a given structure with the radical of a specified substituent including, but not limited to: hydroxyl, hydroxyalkyl, alkoxy, halogen, alkyl, nitro, silyl, acyl, acyloxy, aryl, cycloalkyl, heterocyclyl, amino, aminoalkyl, cyano, haloalkyl, haloalkoxy, —OCO—CH₂—O-alkyl, —OP(O)(O-alkyl)₂ or —CH₂—OP(O)(O-alkyl)₂. Preferably, “optionally substituted” refers to the replacement of one to four hydrogen radicals in a given structure with the substituents mentioned above. More preferably, one to three hydrogen radicals are replaced by the substituents as mentioned above. It is understood that the substituent can be further substituted.

Articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

As used herein, the term “alkyl” refers to saturated aliphatic groups, including but not limited to C₁-C₁₀ straight-chain alkyl groups or C₁-C₁₀ branched-chain alkyl groups. Preferably, the “alkyl” group refers to C₁-C₆ straight-chain alkyl groups or C₁-C₆ branched-chain alkyl groups. Most preferably, the “alkyl” group refers to C₁-C₄ straight-chain alkyl groups or C₁-C₄ branched-chain alkyl groups. Examples of “alkyl” include, but are not limited to, methyl, ethyl, 1-propyl, 2-propyl, n-butyl, sec-butyl, tert-butyl, 1-pentyl, 2-pentyl, 3-pentyl, neo-pentyl, 1-hexyl, 2-hexyl, 3-hexyl, 1-heptyl, 2-heptyl, 3-heptyl, 4-heptyl, 1-octyl, 2-octyl, 3-octyl or 4-octyl and the like. The “alkyl” group may be optionally substituted.

The term “acyl” is art-recognized and refers to a group represented by the general formula hydrocarbylC(O)—, preferably alkylC(O)—.

The term “acylamino” is art-recognized and refers to an amino group substituted with an acyl group and may be represented, for example, by the formula hydrocarbylC(O)NH—.

The term “acyloxy” is art-recognized and refers to a group represented by the general formula hydrocarbylC(O)O—, preferably alkylC(O)O—.

The term “alkoxy” refers to an alkyl group having an oxygen attached thereto. Representative alkoxy groups include methoxy, ethoxy, propoxy, tert-butoxy and the like.

The term “alkoxyalkyl” refers to an alkyl group substituted with an alkoxy group and may be represented by the general formula alkyl-O-alkyl.

The term “alkyl” refers to saturated aliphatic groups, including straight-chain alkyl groups, branched-chain alkyl groups, cycloalkyl (alicyclic) groups, alkyl-substituted cycloalkyl groups, and cycloalkyl-substituted alkyl groups. In preferred embodiments, a straight chain or branched chain alkyl has 30 or fewer carbon atoms in its backbone (e.g., C₁₋₃₀ for straight chains, C₃₋₃₀ for branched chains), and more preferably 20 or fewer.

Moreover, the term “alkyl” as used throughout the specification, examples, and claims is intended to include both unsubstituted and substituted alkyl groups, the latter of which refers to alkyl moieties having substituents replacing a hydrogen on one or more carbons of the hydrocarbon backbone, including haloalkyl groups such as trifluoromethyl and 2,2,2-trifluoroethyl, etc.

The term “C_{x-y}” or “Cₓ-C_{y}”, when used in conjunction with a chemical moiety, such as, acyl, acyloxy, alkyl, alkenyl, alkynyl, or alkoxy is meant to include groups that contain from x to y carbons in the chain. C₀alkyl indicates a hydrogen where the group is in a terminal position, a bond if internal. A C₁₋₆alkyl group, for example, contains from one to six carbon atoms in the chain.

The term “alkylamino”, as used herein, refers to an amino group substituted with at least one alkyl group.

The term “alkylthio”, as used herein, refers to a thiol group substituted with an alkyl group and may be represented by the general formula alkylS—.

The term “amide”, as used herein, refers to a group

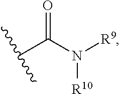

wherein R⁹ and R¹⁰ each independently represent a hydrogen or hydrocarbyl group, or R⁹ and R¹⁰ taken together with the N atom to which they are attached complete a heterocycle having from 4 to 8 atoms in the ring structure. If an amide is drawn without depicting a group required by valence on the constituent nitrogen atom (e.g., as in —C(O)—NR⁹), then a hydrogen atom is implied and understood to be present on the nitrogen atom to satisfy the aforementioned valence requirement (e.g., as in —C(O)—NHR⁹).

The terms “amine” and “amino” are art-recognized and refer to both unsubstituted and substituted amines and salts thereof, e.g., a moiety that can be represented by

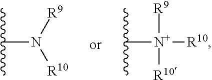

wherein R⁹, R¹⁰, and R¹⁰′ each independently represent a hydrogen or a hydrocarbyl group, or R⁹ and R¹⁰ taken together with the N atom to which they are attached complete a heterocycle having from 4 to 8 atoms in the ring structure.

The term “aminoalkyl”, as used herein, refers to an alkyl group substituted with an amino group.

The term “aralkyl”, as used herein, refers to an alkyl group substituted with an aryl group.

The term “aryl” as used herein include substituted or unsubstituted single-ring aromatic groups in which each atom of the ring is carbon. Preferably, the ring is a 5- to 7-membered ring, more preferably a 6-membered ring. The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Aryl groups include benzene, naphthalene, phenanthrene, phenol, aniline, and the like.

The term “carbamate” is art-recognized and refers to a group

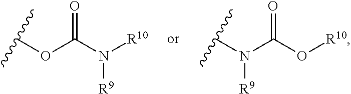

wherein R⁹ and R¹⁰ independently represent hydrogen or a hydrocarbyl group.

The term “carbocyclylalkyl”, as used herein, refers to an alkyl group substituted with a carbocycle group.

The term “carbocycle” includes 5-7 membered monocyclic and 8-12 membered bicyclic rings. Each ring of a bicyclic carbocycle may be selected from saturated, unsaturated and aromatic rings. Carbocycle includes bicyclic molecules in which one, two or three or more atoms are shared between the two rings. The term “fused carbocycle” refers to a bicyclic carbocycle in which each of the rings shares two adjacent atoms with the other ring. Each ring of a fused carbocycle may be selected from saturated, unsaturated and aromatic rings. In an exemplary embodiment, an aromatic ring, e.g., phenyl, may be fused to a saturated or unsaturated ring, e.g., cyclohexane, cyclopentane, or cyclohexene. Any combination of saturated, unsaturated and aromatic bicyclic rings, as valence permits, is included in the definition of carbocyclic. Exemplary “carbocycles” include cyclopentane, cyclohexane, bicyclo[2.2.1]heptane, 1,5-cyclooctadiene, 1,2,3,4-tetrahydronaphthalene, bicyclo[4.2.0]oct-3-ene, naphthalene and adamantane. Exemplary fused carbocycles include decalin, naphthalene, 1,2,3,4-tetrahydronaphthalene, bicyclo[4.2.0]octane, 4,5,6,7-tetrahydro-1H-indene and bicyclo[4.1.0]hept-3-ene. “Carbocycles” may be substituted at any one or more positions capable of bearing a hydrogen atom.

The term “carbocyclylalkyl”, as used herein, refers to an alkyl group substituted with a carbocycle group.

The term “carbonate” is art-recognized and refers to a group —OCO₂—.

The term “carboxy”, as used herein, refers to a group represented by the formula —CO₂H.

The term “ester”, as used herein, refers to a group —C(O)OR⁹ wherein R⁹ represents a hydrocarbyl group.

The term “ether”, as used herein, refers to a hydrocarbyl group linked through an oxygen to another hydrocarbyl group. Accordingly, an ether substituent of a hydrocarbyl group may be hydrocarbyl-O—. Ethers may be either symmetrical or unsymmetrical.

Examples of ethers include, but are not limited to, heterocycle-O-heterocycle and aryl-O-heterocycle. Ethers include “alkoxyalkyl” groups, which may be represented by the general formula alkyl-O-alkyl.

The terms “halo” and “halogen” as used herein means halogen and includes chloro, fluoro, bromo, and iodo.

The terms “hetaralkyl” and “heteroaralkyl”, as used herein, refers to an alkyl group substituted with a hetaryl group.

The terms “heteroaryl” and “hetaryl” include substituted or unsubstituted aromatic single ring structures, preferably 5- to 7-membered rings, more preferably 5- to 6-membered rings, whose ring structures include at least one heteroatom, preferably one to four heteroatoms, more preferably one or two heteroatoms. The terms “heteroaryl” and “hetaryl” also include polycyclic ring systems having two or more cyclic rings in which two or more carbons are common to two adjoining rings wherein at least one of the rings is heteroaromatic, e.g., the other cyclic rings can be cycloalkyls, cycloalkenyls, cycloalkynyls, aryls, heteroaryls, and/or heterocyclyls. Heteroaryl groups include, for example, pyrrole, furan, thiophene, imidazole, oxazole, thiazole, pyrazole, pyridine, pyrazine, pyridazine, pyrimidine, and the like.

The term “heteroatom” as used herein means an atom of any element other than carbon or hydrogen. Preferred heteroatoms are nitrogen, oxygen, and sulfur.

The term “heterocyclylalkyl”, as used herein, refers to an alkyl group substituted with a heterocycle group.

The terms “heterocyclyl”, “heterocycle”, and “heterocyclic” refer to substituted or unsubstituted non-aromatic ring structures, preferably 3- to 10-membered rings, more preferably 3- to 7-membered rings, whose ring structures include at least one heteroatom, preferably one to four heteroatoms, more preferably one or two heteroatoms. The terms “heterocyclyl” and “heterocyclic” also include polycyclic ring systems having two or more cyclic rings in which two or more carbons are common to two adjoining rings wherein at least one of the rings is heterocyclic, e.g., the other cyclic rings can be cycloalkyls, cycloalkenyls, cycloalkynyls, aryls, heteroaryls, and/or heterocyclyls. Heterocyclyl groups include, for example, piperidine, piperazine, pyrrolidine, morpholine, lactones, lactams, and the like.

The term “hydrocarbyl”, as used herein, refers to a group that is bonded through a carbon atom that does not have a ═O or ═S substituent, and typically has at least one carbon-hydrogen bond and a primarily carbon backbone, but may optionally include heteroatoms. Thus, groups like methyl, ethoxyethyl, 2-pyridyl, and even trifluoromethyl are considered to be hydrocarbyl for the purposes of this application, but substituents such as acetyl (which has a ═O substituent on the linking carbon) and ethoxy (which is linked through oxygen, not carbon) are not. Hydrocarbyl groups include, but are not limited to aryl, heteroaryl, carbocycle, heterocycle, alkyl, alkenyl, alkynyl, and combinations thereof.

The term “hydroxyalkyl”, as used herein, refers to an alkyl group substituted with a hydroxy group.

The term “lower” when used in conjunction with a chemical moiety, such as, acyl, acyloxy, alkyl, alkenyl, alkynyl, or alkoxy is meant to include groups where there are ten or fewer atoms in the substituent, preferably six or fewer. A “lower alkyl”, for example, refers to an alkyl group that contains ten or fewer carbon atoms, preferably six or fewer. In certain embodiments, acyl, acyloxy, alkyl, alkenyl, alkynyl, or alkoxy substituents defined herein are respectively lower acyl, lower acyloxy, lower alkyl, lower alkenyl, lower alkynyl, or lower alkoxy, whether they appear alone or in combination with other substituents, such as in the recitations hydroxyalkyl and aralkyl (in which case, for example, the atoms within the aryl group are not counted when counting the carbon atoms in the alkyl substituent).

The terms “polycyclyl”, “polycycle”, and “polycyclic” refer to two or more rings (e.g., cycloalkyls, cycloalkenyls, cycloalkynyls, aryls, heteroaryls, and/or heterocyclyls) in which two or more atoms are common to two adjoining rings, e.g., the rings are “fused rings”. Each of the rings of the polycycle can be substituted or unsubstituted. In certain embodiments, each ring of the polycycle contains from 3 to 10 atoms in the ring, preferably from 5 to 7.

The term “sulfate” is art-recognized and refers to the group —OSO₃H, or a pharmaceutically acceptable salt thereof.

The term “sulfonamide” is art-recognized and refers to the group represented by the general formulae

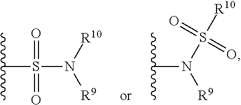

wherein R⁹ and R¹⁰ independently represents hydrogen or hydrocarbyl.

The term “sulfoxide” is art-recognized and refers to the group —S(O)—.

The term “sulfonate” is art-recognized and refers to the group SO₃H, or a pharmaceutically acceptable salt thereof.

The term “sulfone” is art-recognized and refers to the group —S(O)₂—.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate.

The term “thioalkyl”, as used herein, refers to an alkyl group substituted with a thiol group.

The term “thioester”, as used herein, refers to a group —C(O)SR⁹ or —SC(O)R⁹
wherein R⁹ represents a hydrocarbyl.

The term “thioether”, as used herein, is equivalent to an ether, wherein the oxygen is replaced with a sulfur.

The term “urea” is art-recognized and may be represented by the general formula

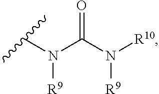

wherein R⁹ and R¹⁰ independently represent hydrogen or a hydrocarbyl.

The term “modulate” as used herein includes the inhibition or suppression of a function or activity (such as cell proliferation) as well as the enhancement of a function or activity.

The phrase “pharmaceutically acceptable” is art-recognized. In certain embodiments, the term includes compositions, excipients, adjuvants, polymers and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Salt” is used herein to refer to an acid addition salt or a basic addition salt.

Many of the lipidoid compositions (e.g., nanoparticles) useful in the methods and compositions of this disclosure have at least one stereogenic center in their structure. This stereogenic center may be present in a R or a S configuration, said R and S notation is used in correspondence with the rules described in Pure Appl. Chem. (1976), 45, 11-30. The disclosure contemplates all stereoisomeric forms such as enantiomeric and diastereoisomeric forms of the compounds, salts, prodrugs or mixtures thereof (including all possible mixtures of stereoisomers). See, e.g., WO 01/062726.

Furthermore, certain compounds which contain alkenyl groups may exist as Z (zusammen) or E (entgegen) isomers. In each instance, the disclosure includes both mixture and separate individual isomers. Some of the lipidoid compositions (e.g., nanoparticles) may also comprise chemical compound which exist in tautomeric forms. Such forms, although not explicitly indicated in the formulae described herein, are intended to be included within the scope of the present disclosure.

“Pharmaceutically acceptable” means approved or approvable by a regulatory agency of the Federal or a state government or the corresponding agency in countries other than the United States, or that is listed in the U.S. Pharmacopoeia or other generally recognized pharmacopoeia for use in animals, and more particularly, in humans.

“Pharmaceutically acceptable salt” refers to a salt of a compound of the invention that is pharmaceutically acceptable and that possesses the desired pharmacological activity of the parent compound. In particular, such salts are non-toxic may be inorganic or organic acid addition salts and base addition salts. Specifically, such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, chlorobenzenesulfonic acid, 2-naphthalenesulfonic acid, 4-toluenesulfonic acid, camphorsulfonic acid, 4-methylbicyclo [2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, N-methylglucamine and the like. Salts further include, by way of example only, sodium potassium, calcium, magnesium, ammonium, tetraalkylammonium, and the like; and when the compound contains a basic functionality, salts of nontoxic organic or inorganic acids, such as hydrochloride, hydrobromide, tartrate, mesylate, acetate, maleate, oxalate and the like.

The term “pharmaceutically acceptable basic addition salt” as used herein means any non-toxic organic or inorganic base addition salt of any acid compounds disclosed herein. Illustrative inorganic bases which form suitable salts include lithium, sodium, potassium, calcium, magnesium, or barium hydroxide. Illustrative organic bases which form suitable salts include aliphatic, alicyclic, or aromatic organic amines such as methylamine, trimethylamine and picoline or ammonia. The selection of the appropriate salt will be known to a person skilled in the art.

The term “pharmaceutically acceptable cation” refers to an acceptable cationic counterion of an acidic functional group. Such cations are exemplified by sodium, potassium, calcium, magnesium, ammonium, tetraalkylammonium cations, and the like (see, e. g., Berge, et al., J. Pharm. Sci. 66 (1):1-79 (January 77).

“Pharmaceutically acceptable vehicle” refers to a diluent, adjuvant, excipient or carrier with which a compound of the invention is administered.

“Pharmaceutically acceptable metabolically cleavable group” refers to a group that is cleaved in vivo to yield the parent molecule of the structural formula indicated herein. Examples of metabolically cleavable groups include —COR, —COOR, —CONRR and —CH₂OR radicals, where R is selected independently at each occurrence from alkyl, trialkylsilyl, carbocyclic aryl or carbocyclic aryl substituted with one or more of alkyl, halogen, hydroxy or alkoxy. Specific examples of representative metabolically cleavable groups include acetyl, methoxycarbonyl, benzoyl, methoxymethyl and trimethylsilyl groups.

The phrase “pharmaceutically acceptable carrier” as used herein means a pharmaceutically acceptable material, composition or vehicle, such as a liquid or solid filter, diluent, excipient, solvent or encapsulating material useful for formulating a drug for medicinal or therapeutic use.

“Prodrugs” refers to compounds, including derivatives of the compounds of the invention, which have cleavable groups and become by solvolysis or under physiological conditions the compounds of the invention which are pharmaceutically active in vivo. Such examples include, but are not limited to, choline ester derivatives and the like, N-alkylmorpholine esters and the like. Other derivatives of the compounds of this invention have activity in both their acid and acid derivative forms, but in the acid sensitive form often offers advantages of solubility, tissue compatibility, or delayed release in the mammalian organism (see, Bundgard, H., Design of Prodrugs, pp. 7-9, 21-24, Elsevier, Amsterdam 1985). Prodrugs include acid derivatives well known to practitioners of the art, such as, for example, esters prepared by reaction of the parent acid with a suitable alcohol, or amides prepared by reaction of the parent acid compound with a substituted or unsubstituted amine, or acid anhydrides, or mixed anhydrides. Simple aliphatic or aromatic esters, amides and anhydrides derived from acidic groups pendant on the compounds of this invention are particular prodrugs. In some cases it is desirable to prepare double ester type prodrugs such as (acyloxy)alkylesters or (alkoxycarbonyl)oxy)alkylesters. Particularly the C₁-C₈ alkyl, C₂-C₈ alkenyl, C₂-C₈ alkynyl, aryl, C₇-C₁₂ substituted aryl, and C₇-C₁₂ arylalkyl esters of the compounds of the invention.

“Solvate” refers to forms of the compound that are associated with a solvent or water (also referred to as “hydrate”), usually by a solvolysis reaction. This physical association includes hydrogen bonding. Conventional solvents include water, ethanol, acetic acid and the like. The compounds of the invention may be prepared e.g., in crystalline form and may be solvated or hydrated. Suitable solvates include pharmaceutically acceptable solvates, such as hydrates, and further include both stoichiometric solvates and non-stoichiometric solvates. In certain instances, the solvate will be capable of isolation, for example when one or more solvent molecules are incorporated in the crystal lattice of the crystalline solid. “Solvate” encompasses both solution-phase and isolable solvates. Representative solvates include hydrates, ethanolates and methanolates.

A “subject” to which administration is contemplated includes, but is not limited to, humans (i.e., a male or female of any age group, e.g., a pediatric subject (e.g, infant, child, adolescent) or adult subject (e.g., young adult, middle aged adult or senior adult) and/or a non-human animal, e.g., a mammal such as primates (e.g., cynomolgus monkeys, rhesus monkeys), cattle, pigs, horses, sheep, goats, rodents, cats, and/or dogs. In certain embodiments, the subject is a human. In certain embodiments, the subject is a non-human animal. The terms “human,” “patient,” and “subject” are used interchangeably herein.

An “effective amount” means the amount of a compound that, when administered to a subject for treating or preventing a disease, is sufficient to effect such treatment or prevention. The “effective amount” can vary depending on the compound, the disease and its severity, and the age, weight, etc., of the subject to be treated. A “therapeutically effective amount” refers to the effective amount for therapeutic treatment. A “prophylactically effective amount” refers to the effective amount for prophylactic treatment.

“Preventing” or “prevention” or “prophylactic treatment” refers to a reduction in risk of acquiring or developing a disease or disorder (i.e., causing at least one of the clinical symptoms of the disease not to develop in a subject not yet exposed to a disease-causing agent, or predisposed to the disease in advance of disease onset.

The term “prophylaxis” is related to “prevention,” and refers to a measure or procedure the purpose of which is to prevent, rather than to treat or cure a disease. Non limiting examples of prophylactic measures may include the administration of vaccines; the administration of low molecular weight heparin to hospital patients at risk for thrombosis due, for example, to immobilization, and the administration of an anti-malarial agent such as chloroquine, in advance of a visit to a geographical region where malaria is endemic or the risk of contracting malaria is high.

“Treating” or “treatment” or “therapeutic treatment” of any disease or disorder refers, in one embodiment, to ameliorating the disease or disorder (i.e., arresting the disease or reducing the manifestation, extent or severity of at least one of the clinical symptoms thereof). In another embodiment “treating” or “treatment” refers to ameliorating at least one physical parameter, which may not be discernible by the subject. In yet another embodiment, “treating” or “treatment” refers to modulating the disease or disorder, either physically, (e.g., stabilization of a discernible symptom), physiologically, (e.g., stabilization of a physical parameter), or both. In a further embodiment, “treating” or “treatment” relates to slowing the progression of the disease.

“Administering” or “administration of” a substance, a compound or an agent to a subject can be carried out using one of a variety of methods known to those skilled in the art. For example, a compound or an agent can be administered, intravenously, arterially, intradermally, intramuscularly, intraperitoneally, subcutaneously, ocularly, sublingually, orally (by ingestion), intranasally (by inhalation), intraspinally, intracerebrally, and transdermally (by absorption, e.g., through a skin duct). A compound or agent can also appropriately be introduced by rechargeable or biodegradable polymeric devices or other devices, e.g., patches and pumps, or formulations, which provide for the extended, slow or controlled release of the compound or agent. Administering can also be performed, for example, once, a plurality of times, and/or over one or more extended periods. Appropriate methods of administering a substance, a compound or an agent to a subject will also depend, for example, on the age and/or the physical condition of the subject and the chemical and biological properties of the compound or agent (e.g., solubility, digestibility, bioavailability, stability and toxicity). In some embodiments, a compound or an agent is administered orally, e.g., to a subject by ingestion. In some embodiments, the orally administered compound or agent is in an extended release or slow release formulation, or administered using a device for such slow or extended release.

As used herein, the phrase “conjoint administration” refers to any form of administration of two or more different therapeutic agents such that the second agent is administered while the previously administered therapeutic agent is still effective in the body (e.g., the two agents are simultaneously effective in the patient, which may include synergistic effects of the two agents). For example, the different therapeutic compounds can be administered either in the same formulation or in separate formulations, either concomitantly or sequentially. Thus, an individual who receives such treatment can benefit from a combined effect of different therapeutic agents.

A “therapeutically effective amount” or a “therapeutically effective dose” of a drug or agent is an amount of a drug or an agent that, when administered to a subject will have the intended therapeutic effect. The full therapeutic effect does not necessarily occur by administration of one dose, and may occur only after administration of a series of doses. Thus, a therapeutically effective amount may be administered in one or more administrations. The precise effective amount needed for a subject will depend upon, for example, the subject's size, health and age, and the nature and extent of the condition being treated, such as cancer or MDS. The skilled worker can readily determine the effective amount for a given situation by routine experimentation.

As used herein, the term “isotopic variant” refers to a compound that contains unnatural proportions of isotopes at one or more of the atoms that constitute such compound. For example, an “isotopic variant” of a compound can contain one or more non-radioactive isotopes, such as for example, deuterium (²H or D), carbon-13 (¹³C), nitrogen-15 (¹⁵N), or the like. It will be understood that, in a compound where such isotopic substitution is made, the following atoms, where present, may vary, so that for example, any hydrogen may be “²H/D, any carbon may be ¹³C, or any nitrogen may be ¹⁵N, and that the presence and placement of such atoms may be determined within the skill of the art. Likewise, the invention may include the preparation of isotopic variants with radioisotopes, in the instance for example, where the resulting compounds may be used for drug and/or substrate tissue distribution studies. The radioactive isotopes tritium, i.e., ³H, and carbon-14, i.e., ¹⁴C, are particularly useful for this purpose in view of their ease of incorporation and ready means of detection. Further, com pounds may be prepared that are substituted with positron emitting isotopes, such as ¹¹C, ¹⁸F, ¹⁵O and ¹³N, and would be useful in Positron Emission Topography (PET) studies for examining substrate receptor occupancy. All isotopic variants of the compounds provided herein, radioactive or not, are intended to be encompassed within the scope of the invention.

It is also to be understood that compounds that have the same molecular formula but differ in the nature or sequence of bonding of their atoms or the arrangement of their atoms in space are termed “isomers.” Isomers that differ in the arrangement of their atoms in space are termed “stereoisomers.”

Stereoisomers that are not mirror images of one another are termed “diastereomers” and those that are non-superimposable mirror images of each other are termed “enantiomers.” When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+)- or (−)-isomers respectively). A chiral compound can exist as either individual enantiomer or as a mixture thereof. A mixture containing equal proportions of the enantiomers is called a “racemic mixture”.

“Tautomers” refer to compounds that are interchangeable forms of a particular compound structure, and that vary in the displacement of hydrogen atoms and electrons. Thus, two structures may be in equilibrium through the movement of it electrons and an atom (usually H). For example, enols and ketones are tautomers because they are rapidly interconverted by treatment with either acid or base. Another example of tautomerism is the aci- and nitro-forms of phenylnitromethane, that are likewise formed by treatment with acid or base. Tautomeric forms may be relevant to the attainment of the optimal chemical reactivity and biological activity of a compound of interest.

As used herein a pure enantiomeric compound is substantially free from other enantiomers or stereoisomers of the compound (i.e., in enantiomeric excess). In other words, an “S” form of the compound is substantially free from the “R” form of the compound and is, thus, in enantiomeric excess of the “R” form. The term “enantiomerically pure” or “pure enantiomer” denotes that the compound comprises more than 95% by weight, more than 96% by weight, more than 97% by weight, more than 98% by weight, more than 98.5% by weight, more than 99% by weight, more than 99.2% by weight, more than 99.5% by weight, more than 99.6% by weight, more than 99.7% by weight, more than 99.8% by weight or more than 99.9% by weight, of the enantiomer. In certain embodiments, the weights are based upon total weight of all enantiomers or stereoisomers of the compound.

As used herein and unless otherwise indicated, the term “enantiomerically pure R-compound” refers to at least about 95% by weight R-compound and at most about 5% by weight S-compound, at least about 99% by weight R-compound and at most about 1% by weight S-compound, or at least about 99.9% by weight R-compound and at most about 0.1% by weight S-compound. In certain embodiments, the weights are based upon total weight of compound.

As used herein and unless otherwise indicated, the term “enantiomerically pure S-compound” or “S-compound” refers to at least about 95% by weight S-compound and at most about 5% by weight R-compound, at least about 99% by weight S-compound and at most about 1% by weight R-compound or at least about 99.9% by weight S-compound and at most about 0.1% by weight R-compound. In certain embodiments, the weights are based upon total weight of compound.

In the compositions provided herein, an enantiomerically pure compound or a pharmaceutically acceptable salt, solvate, hydrate or prodrug thereof can be present with other active or inactive ingredients. For example, a pharmaceutical composition comprising enantiomerically pure R-compound can comprise, for example, about 90% excipient and about 10% enantiomerically pure R-compound. In certain embodiments, the enantiomerically pure R-compound in such compositions can, for example, comprise at least about 95% by weight R-compound and at most about 5% by weight S-compound, by total weight of the compound. For example, a pharmaceutical composition comprising enantiomerically pure S-compound can comprise, for example, about 90% excipient and about 10% enantiomerically pure S-compound. In certain embodiments, the enantiomerically pure S-compound in such compositions can, for example, comprise, at least about 95% by weight S-compound and at most about 5% by weight R-compound, by total weight of the compound. In certain embodiments, the active ingredient can be formulated with little or no excipient or carrier.

The compounds of this invention may possess one or more asymmetric centers; such compounds can therefore be produced as individual (R)- or (S)-stereoisomers or as mixtures thereof.

Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art.

One having ordinary skill in the art of organic synthesis will recognize that the maximum number of heteroatoms in a stable, chemically feasible heterocyclic ring, whether it is aromatic or non-aromatic, is determined by the size of the ring, the degree of unsaturation and the valence of the heteroatoms. In general, a heterocyclic ring may have one to four heteroatoms so long as the heteroaromatic ring is chemically feasible and stable.

### List of Embodiments

The following list of embodiments of the invention are to be considered as disclosing various features of the invention, which features can be considered to be specific to the particular embodiment under which they are discussed, or which are combinable with the various other features as listed in other embodiments. Thus, simply because a feature is discussed under one particular embodiment does not necessarily limit the use of that feature to that embodiment.

Embodiment 1. A composition comprising: a pharmaceutical agent assembled with a lipid composition, wherein the lipid composition comprises a lipidoid having structural Formula (I):

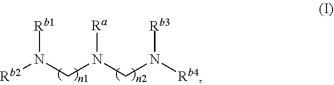

or a pharmaceutically acceptable salt thereof, wherein:
R^{a} is an alkyl;
n1 and n2 are each independently 1, 2, 3, or 4; and
R^{b1}, R^{b2}, R^{b3} and R^{b4} are each independently H,

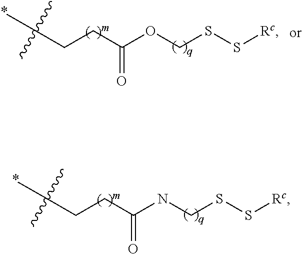

wherein:
* indicates the point of attachment to N;
each m is independently 1, 2, 3, 4, or 5;
each q is independently 1, 2, 3, 4, or 5; and
each R^{c} is independently an alkyl (e.g., C₄-C₂₀) or an alkenyl (e.g., C₄-C₂₀); and
wherein at least one of R^{b1}, R^{b2}, R^{b3} and R^{b4} is not H.

Embodiment 2. The composition of embodiment 1, wherein the composition is for preferential delivery to a target organ or a target cell

Embodiment 3. The composition of embodiment 1, wherein the composition is for modifying an expression profile of a target gene or a gene product thereof in the target organ or the target cell

Embodiment 4. The composition of embodiment 3, wherein the target gene or the gene product is a target protein or a functional variant thereof, or a target transcript.

Embodiment 5. The composition of any one of embodiments 1-4, wherein the pharmaceutical agent is a therapeutic agent, a gene modulating agent, or a vaccine.

Embodiment 6. The composition of embodiment 1-5, wherein R^{a} C₁-C₄ alkyl.

Embodiment 7. The composition of embodiment 1-6, wherein R^{a} is C₁-C₃ alkyl

Embodiment 8. The composition of any one of embodiments 1-7, wherein R^{a} is C₁ alkyl.

Embodiment 9. The composition of any one of embodiments 1-8, wherein n1 is 1, 2 or 3.

Embodiment 10. The composition of embodiment 9, wherein n1 is 2 or 3.

Embodiment 11. The composition of any one of embodiments 1-10, wherein n2 is 1, 2 or 3.

Embodiment 12. The composition of embodiment 11, wherein n2 is 2 or 3.

Embodiment 13. The composition of any one of embodiments 1-12, wherein n1 and n2 are identical.

Embodiment 14. The composition of any one of embodiments 1-13, wherein at least two of R^{b1}, R^{b2}, R^{b3} and R^{b4} are not H.

Embodiment 15. The composition of any one of embodiments 1-14, wherein at least three of R^{b1}, R^{b2}, R^{b3} and R^{b4} are not H.

Embodiment 16. The composition of any one of embodiments 1-13, wherein none of R^{b1}, R^{b2}, R^{b3} and R^{b4} is H.

Embodiment 17. The composition of any one of embodiments 1-13, wherein R^{b1}, R^{b2}, R^{b3} and R^{b4} are each independently

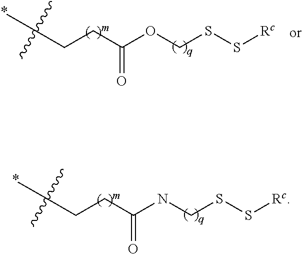

Embodiment 18. The composition of any one of embodiments 1-13, wherein R^{b1}, R^{b2}, R^{b3} and R^{b4} are each independently

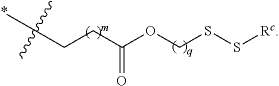

Embodiment 19. The composition of any one of embodiments 1-13, wherein R^{b1}, R^{b2}, R^{b3} and R^{b4} are each independently

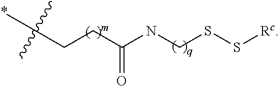

Embodiment 20. The composition of any one of embodiments 1-19, wherein each R^{c} is independently C₄-C₁₆ alkyl or C₄₋₁₆ alkenyl.

Embodiment 21. The composition of any one of embodiments 1-20, wherein R^{c} is independently C₆-C₁₂ alkyl or C₆-C₁₂ alkenyl.

Embodiment 22. The composition of any one of embodiments 1-19, wherein each R^{c} is independently C₄-C₂₀ alkyl (e.g., C₄-C₁₆ alkyl, such as C₄-C₁₂ alkyl).

Embodiment 23. The composition of any one of embodiments 1-22, wherein each R^{c} is independently C₄-C₁₆ alkyl.

Embodiment 24. The compositions of any one of embodiments 1-23, wherein each R^{c} is independently C₄-C₁₂ alkyl.

Embodiment 25. The composition of any one of embodiments 1-22, wherein each m is independently 1, 2, or 3.

Embodiment 26. The composition of embodiment 25, wherein each m is independently 1 or 2.

Embodiment 27. The composition of embodiment 25, wherein each m is 1.

Embodiment 28. The composition of any one of embodiments 1-27, wherein each q is independently 1, 2, 3, or 4.

Embodiment 29. The composition of embodiment 28, wherein each q is independently 1, 2, or 3.

Embodiment 30. The composition of embodiment 28, wherein each q is 2.

Embodiment 31. The composition of any one of embodiments 1-30, wherein the lipidoid of Formula (I), or the pharmaceutically acceptable salt thereof, is present in the lipid composition at a molar percentage of no more than about 60%.

Embodiment 32. The composition of any one of embodiments 1-31, wherein the lipidoid of Formula (I), or the pharmaceutically acceptable salt thereof, is present in the lipid composition at a molar percentage of no more than about 50%.

Embodiment 33. The composition of any one of embodiments 1-32, wherein the lipidoid of Formula (I), or the pharmaceutically acceptable salt thereof, is present in the lipid composition at a molar percentage of no more than about 40%.

Embodiment 34. The composition of any one of embodiments 1-33, wherein the lipid composition further comprises a steroid or steroid derivative.

Embodiment 35. The composition of any of embodiments 1-34, wherein the lipid composition further comprises a cholesterol or cholesterol derivative.

Embodiment 36. The composition of embodiment 35, wherein the steroid or steroid derivative is present in the lipid composition at a molar percentage of no more than about 50%.

Embodiment 37. The composition of any one of embodiments 1-36, wherein the lipid composition further comprises a polymer-conjugated lipid.

Embodiment 38. The composition of any of embodiments 1-37, wherein the lipid composition further comprises a poly(ethylene glycol) (PEG) conjugated lipid.

Embodiment 39. The composition of embodiment 37 or 38, wherein the polymer-conjugated lipid is present in the lipid composition at a molar percentage of no more than about 10%.

Embodiment 40. The composition of any one of embodiments 1-39, wherein the lipid composition further comprises a phospholipid.

Embodiment 41. The composition of any one of embodiments 1-40, wherein the lipid composition further comprises a phosphoethanolamine lipid or a phosphocholine lipid.

Embodiment 42. The composition of embodiment 40, wherein the phospholipid is present in the lipid composition at a molar percentage of no more than about 30%.

Embodiment 43. The composition of any one of embodiments 1-42, wherein the lipidoid of Formula (I), or the pharmaceutically acceptable salt thereof, is present in the composition at a mass or weight ratio to the pharmaceutical agent of about 1:1 to about 200:1.

Embodiment 44. The composition of any one of embodiments 1-43, wherein the lipidoid of Formula (I), or the pharmaceutically acceptable salt thereof, is present in the composition at a mass or weight ratio to the pharmaceutical agent of about 1:1 to about 100:1.

Embodiment 45. The composition of any one of embodiments 1-44, wherein the pharmaceutical agent comprises a polynucleotide.

Embodiment 46. The composition of any one of embodiments 1-44, wherein the pharmaceutical agent is a messenger ribonucleic acid (mRNA), an oligonucleotide, a polypeptide, an oligopeptide, a small molecule compound, or any combination thereof.

Embodiment 47. The composition of embodiment 46, wherein the polypeptide is a protein.

Embodiment 48. The composition of embodiment 45, wherein the polynucleotide is configured to up regulate or downregulate a target gene or a gene product thereof.

Embodiment 49. The composition of embodiment 48, wherein the target gene or the gene product thereof is a target protein or a functional variant thereof or a target transcript.

Embodiment 50. The composition of any one of embodiments 1-49, wherein the pharmaceutical agent comprises a polynucleotide.

Embodiment 51. The composition of embodiment 50, wherein the polynucleotide is a messenger ribonucleic acid (mRNA) that encodes or is configured to upregulate or downregulate a target gene or a gene product thereof.

Embodiment 52. The composition of embodiment 51, wherein the target gene or the gene product thereof is a target protein or a function variant thereof or a target transcript.

Embodiment 53. The composition of any of one of embodiments 1-52, wherein the target transcript is in a target organ or a target cell.

Embodiment 54. The composition of any one of embodiments 1-53, wherein the pharmaceutical agent comprises:
(a) a gene modulating moiety configured to specifically bind at least a portion of a target gene or a gene product thereof; or
(b) a polynucleotide that encodes the gene modulating moiety of (a).

Embodiment 55. The composition of embodiment 54, wherein the gene modulating moiety comprises a guide nucleic acid configured to complex with at least a portion of the target gene or the gene product thereof, or a polynucleotide sequence that encodes the guide nucleic acid.

Embodiment 56. The composition of embodiment 54 or 55, wherein the target gene or the gene product is a target protein or a functional variant thereof or a target transcript.

Embodiment 57. The composition of any one of embodiments 54-56, wherein the polynucleotide is a messenger ribonucleic acid (mRNA)) that encodes the gene modulating moiety of (a).

Embodiment 58. The composition of any one of embodiments 54-57, wherein the gene modulating moiety comprises a heterologous endonuclease or a polynucleotide comprising a sequence that encodes the heterologous endonuclease.

Embodiment 59. The composition of any one of embodiments 54-58, wherein the heterologous endonuclease is a clustered regularly interspaced short palindromic repeats (CRSIPR)-associated (Cas) nuclease.

Embodiment 60. The composition of any one of embodiments 54-69, wherein the polynucleotide is a messenger ribonucleic acid (mRNA).

Embodiment 61. The composition of embodiment 60, wherein the heterologous endonuclease is present in the gene modulating moiety at a mass or weight ratio to the guide nucleic acid of about 1:20 to about 20:1.

Embodiment 62. The composition of embodiment 61, wherein the heterologous endonuclease is present in the gene modulating moiety at a mass or weight ratio to the guide nucleic acid of about 1:10 to about 10:1.

Embodiment 63. The composition of any one of embodiments 1-62, wherein the target gene or the gene product thereof is specific to or primarily found in a target organ or a target cell of a subject.

Embodiment 64. The composition of embodiment 63, wherein the target gene or the gene product thereof is a target protein or a functional variant thereof or the target transcript.

Embodiment 65. The composition of embodiment 63 or 64, wherein the target gene or the gene product thereof is associated with a disease or disorder of the target organ or the target cell.

Embodiment 66. The composition of any one of embodiments 54-65, wherein the gene modulating moiety is configured to provide a modified expression profile of the target gene or the gene product thereof in a target organ or a target cell of a subject.

Embodiment 67. The composition of any one of embodiments 1-66, wherein the target organ is lung or liver.

Embodiment 68. The composition of any one of embodiments 1-67, wherein the target cell is a lung cell or a liver cell.

Embodiment 69. The composition of any one of embodiments 1-68, wherein the composition is formulated for systemic administration.

Embodiment 70. The composition of any one of embodiments 1-68, wherein the composition is formulated for local administration.

Embodiment 71. A composition according to any one of embodiments 1-70, wherein, in the lipidoid of Formula (I) or the pharmaceutically acceptable salt thereof, R^{b1}, R^{b2}, R^{b3} and R^{b4} are each independently H or

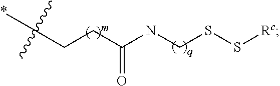

and
at least one of R^{b1}, R^{b2}, R^{b3} and R^{b4} is

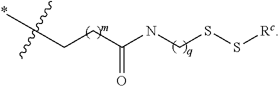

Embodiment 72. The composition of embodiment 71, wherein at least two (e.g., at least three, or all four) of R^{b1}, R^{b2}, R^{b3} and R^{b4} are each independently

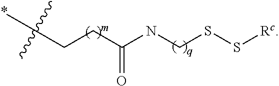

Embodiment 73. The composition of embodiment 72, wherein at least three of R^{b1}, R^{b2}, R^{b3} and R^{b4} are each independently

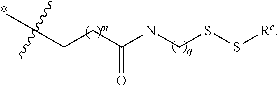

Embodiment 74. The composition of embodiment of 73, wherein all four of R^{b1}, R^{b2}, R^{b3} and R^{b4} are each independently

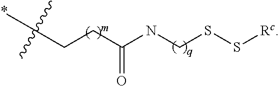

Embodiment 75. The composition of any one of embodiments 71-74, wherein the composition is for preferential delivery of the pharmaceutical agent to a lung.

Embodiment 76. The composition of any one of embodiments 71-75, wherein the lipidoid of Formula (I), or the pharmaceutically acceptable salt thereof, is a lipidoid having structural Formula (II_{A}), (II_{B}), (II_{C}), (II_{D}), or (II_{E}):

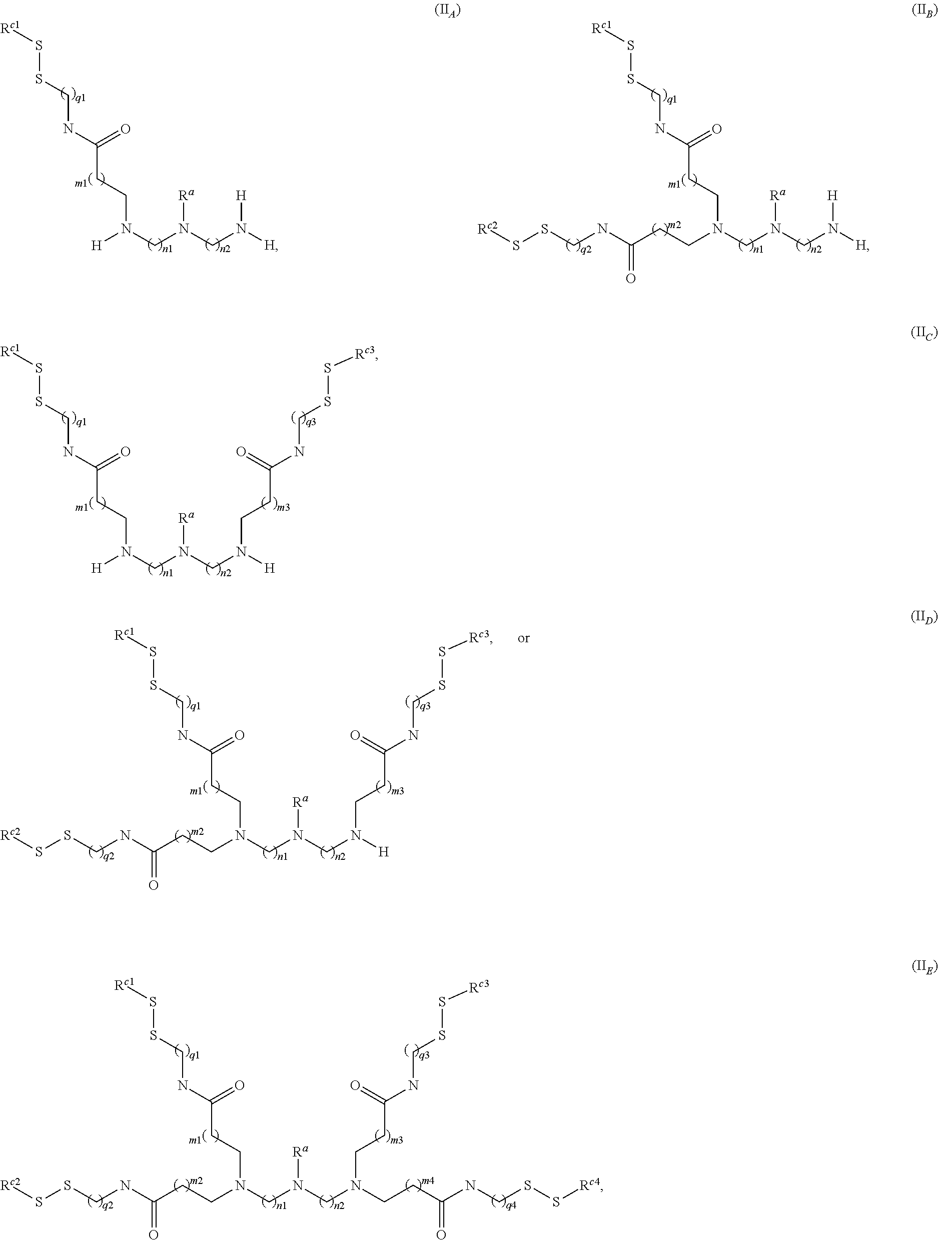

or a pharmaceutically acceptable salt thereof, wherein:
m1, m2, m3, and m4, when present, are each independently 1, 2, or 3;
q1, q2, q3 and q4, when present, are each independently 1, 2, 3, or 4; and
R^{c1}, R^{c2}, R^{c3} and R^{c4}, when present, are each independently C₄-C₂₀ alkyl or C₄-C₂₀ alkenyl.

Embodiment 77. The composition of embodiment 76, wherein the lipidoid of Formula (I), or the pharmaceutically acceptable salt thereof, is the lipidoid of Formula (II_{E}) or the pharmaceutically acceptable salt thereof.

Embodiment 78. The composition of embodiment 76 or 77, wherein R^{c1} is C₄-C₁₆ (e.g., C₆-C₁₂) alkyl or C₄-C₁₆ (e.g., C₆-C₁₂) alkenyl.

Embodiment 79. The composition of embodiment 78, wherein R^{c1} is C₆-C₁₂ alkyl or C₆-C₁₂ alkenyl.

Embodiment 80. The composition of any one of embodiments 76-79, wherein, in Formula (II_{B}), (II_{D}), or (II_{E}), R^{c2} is C₄-C₁₆ (e.g., C₆-C₁₂) alkyl or C₄-C₁₆ (e.g., C₆-C₁₂) alkenyl.

Embodiment 81. The composition of any one of embodiments 76-80, wherein in Formula (II_{B}), (II_{D}), or (II_{E}), R^{c2} is C₆-C₁₂ alkyl or C₆-C₁₂ alkenyl

Embodiment 81. The composition of any one of embodiments 76-81, wherein, in Formula (II_{C}), (II_{D}), or (II_{E}), R^{c3} is C₄-C₁₆ (e.g., C₆-C₁₂) alkyl or C₄-C₁₆ (e.g., C₆-C₁₂) alkenyl.

Embodiment 83. The composition of any one of embodiments 76-82, wherein, in Formula (II_{C}), (II_{D}), or (II_{E}), R^{c3} is C₆-C₁₂ alkyl or C₆-C₁₂ alkenyl

Embodiment 84. The composition of any one of embodiments 76-82, wherein, in Formula (II_{E}), R^{c4} is C₄-C₁₆ (e.g., C₆-C₁₂) alkyl or C₄-C₁₆ (e.g., C₆-C₁₂) alkenyl.

Embodiment 85. The composition of any one of embodiments 76-84, wherein, in Formula (II_{E}), R^{c4} is C₆-C₁₂ alkyl or C₆-C₁₂ alkenyl.

Embodiment 86. The composition of any one of embodiments 76-85, wherein m1 is 1 or 2.

Embodiment 87. The composition of any one of embodiments 76-86, wherein, in Formula (II_{B}), (II_{D}), or (II_{E}), m2 is 1 or 2.

Embodiment 88. The composition of any one of embodiments 76-86, wherein, in Formula (II_{C}), (II_{D}), or (II_{E}), m3 is 1 or 2.

Embodiment 89. The composition of any one of embodiments 76-86, wherein, in Formula (II_{E}), m4 is 1 or 2.

Embodiment 90. The composition of any one of embodiments 76-89, wherein q1 is 1, 2, or 3.

Embodiment 91. The composition of any one of embodiments 76-90, wherein, in Formula (II_{B}), (II_{D}), or (II_{E}), q2 is 1, 2, or 3.

Embodiment 92. The composition of any one of embodiments 76-91, wherein, in Formula (II_{C}), (II_{D}), or (II_{E}), q3 is 1, 2, or 3.

Embodiment 93. The composition of any one of embodiments 76-92, wherein, in Formula (II_{E}), q4 is 1, 2, or 3.

Embodiment 94. The composition of embodiment 71, wherein the lipidoid is selected from:

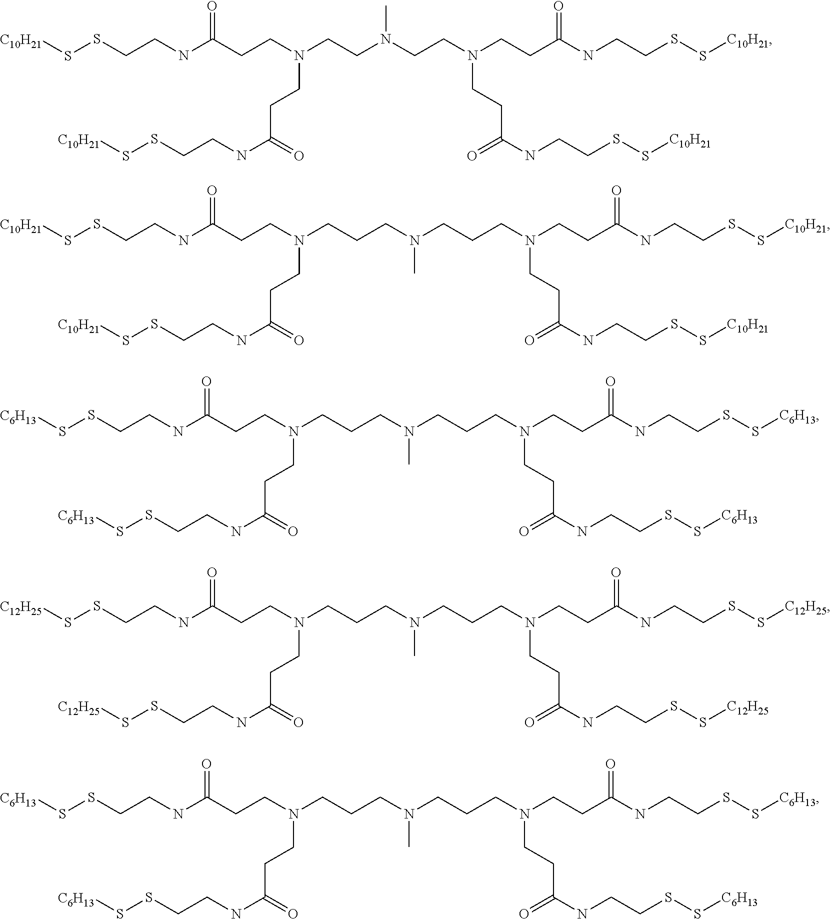

and any pharmaceutically acceptable salt of any of the foregoing.

Embodiment 95. The composition of any one of embodiments 71-94, wherein the lipidoid of Formula (I), (II_{A}), (II_{B}), (II_{C}), (II_{D}), or (II_{E}), or the pharmaceutically acceptable salt thereof, is present in the lipid composition at a molar percentage of about 20% to about 50%.

Embodiment 96. The composition of any one of embodiments 71-95, wherein the lipid composition comprises a steroid or steroid derivative at a molar percentage of about 10% to about 50%.

Embodiment 97. The composition of any one of embodiments 71-96, wherein the lipidoid of Formula (I), (II_{A}), (II_{B}), (II_{C}), (II_{D}), or (II_{E}), or the pharmaceutically acceptable salt thereof, is present in the composition at a mass or weight ratio to the pharmaceutical agent of about 5:1 to about 100:1.

Embodiment 98. The composition of any one of embodiments 71-97, wherein the composition is for a preferential delivery of the pharmaceutical agent to a lung or a lung cell as compared to a delivery to a non-lung organ or a non-lung cell.

Embodiment 99. The composition of embodiment 98, wherein pharmaceutical agent is delivered to a lung or a lung cell in a subject.

Embodiment 100. The composition of embodiment 98, wherein the non-lung organ is a liver, heart, spleen or kidney, and wherein the non-lung cell is a liver cell, a heart cell, a spleen cell, or a kidney cell.

Embodiment 101. The composition of embodiment 98, wherein the pharmaceutical agent encodes or is configured to up-regulate a target gene or a gene product thereof that is specific to or primarily found in the lung or the lung cell.

Embodiment 102. The composition of embodiment 98, wherein the pharmaceutical agent encodes or is configured to down-regulate a target gene or a gene product thereof that is specific to or primarily found in the lung or the lung cell.

Embodiment 103. The composition of any one of embodiments 71-103, wherein the target gene or gene product thereof is a target protein or a functional variant thereof or a target transcript.

Embodiment 104. The composition of embodiment 101, wherein the pharmaceutical agent is configured to provide a modified expression profile of the target gene or the gene product thereof in the lung or the lung cell.

Embodiment 105. The composition of any one of embodiments 98-104, wherein the pharmaceutical agent is associated with a lung disease or disorder.

Embodiment 106. A method for preferential delivery of a pharmaceutical agent to a lung or a lung cell in a subject in need thereof, the method comprising administering the composition according to any one of embodiments 71-105, thereby providing a greater amount (e.g., a 2-fold greater amount) of expression or activity of the pharmaceutical agent in the lung or the lung cell of the subject as compared to that achieved in a non-lung organ or a non-lung cell in the subject.

Embodiment 107. The method of embodiment 106, wherein the method comprises administering the composition according to any one of embodiments 71-105, thereby providing at least a 5-fold greater amount expression or activity of the pharmaceutical agent in the lung or the lung cell of the subject as compared to that achieved in a non-lung organ or a non-lung cell.

Embodiment 108. The method of embodiment 106, wherein the method comprises administering the composition according to any one of embodiments 71-105, thereby providing at least a 10-fold greater amount expression or activity of the pharmaceutical agent in the lung or the lung cell of the subject as compared to that achieved in a non-lung organ or a non-lung cell.

Embodiment 109. The method of any one of embodiments 71-108, wherein the non-lung organ is a liver, heart, spleen, or kidney, and wherein the non-lung cell is a liver cell, a heart cell, a spleen cell, or a kidney cell.

Embodiment 110. A method for preferential delivery of a pharmaceutical agent to a lung or a lung cell in a subject in need thereof, the method comprising administering the composition according to any one of embodiments 71-109, thereby providing a greater amount (e.g., at least about a 2-fold greater amount) of expression or activity of the pharmaceutical agent in the lung or the lung cell of the subject as compared to that achieved with a corresponding reference lipid composition comprising a corresponding reference lipidoid.

Embodiment 111. The method of embodiment 110, wherein the corresponding reference lipidoid has an ester-containing tail.

Embodiment 112. The method of embodiment 106 or 110, wherein the method modulates a greater amount (e.g., at least about 2-fold greater amount) of activity of a target gene or a gene product thereof in the lung or the lung cell of the subject as compared to that achieved with a corresponding reference lipid composition comprising a corresponding reference lipidoid.

Embodiment 113. The method of embodiment 112, wherein the corresponding reference lipidoid has an ester-containing tail.

Embodiment 114. The method of embodiment 112, wherein the method provides a modified expression profile of the target gene or the gene product thereof in the lung or the lung cell of the subject.

Embodiment 115. A composition according to any one of embodiments 1-114, wherein, in the lipidoid of Formula (I) or the pharmaceutically acceptable salt thereof,
R^{b1}, R^{b2}, R^{b3} and R^{b4} are each independently H or

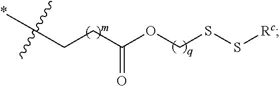

and
at least one of R^{b1}, R^{b2}, R^{b3} and R^{b4} is

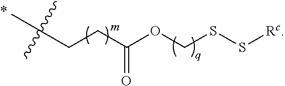

Embodiment 116. The composition of embodiment 115, wherein at least two (e.g., at least three, or all four) of R^{b1}, R^{b2}, R^{b3} and R^{b4} are independently

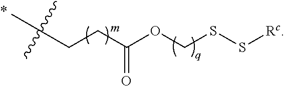

Embodiment 117. The composition of embodiment 116, wherein at least three of R^{b1}, R^{b2}, R^{b3} and R^{b4} are independently

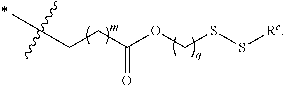

Embodiment 118. The composition of embodiment 117, wherein all four of R^{b1}, R^{b2}, R^{b3} and R^{b4} are independently

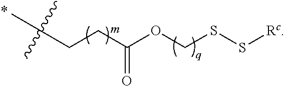

Embodiment 119. The composition of any one of embodiments 115-118, wherein the lipidoid of Formula (I), or the pharmaceutically acceptable salt thereof, is a lipidoid having structural Formula (III_{A}), (III_{B}), (III_{C}), (III_{D}), or (III_{E}):

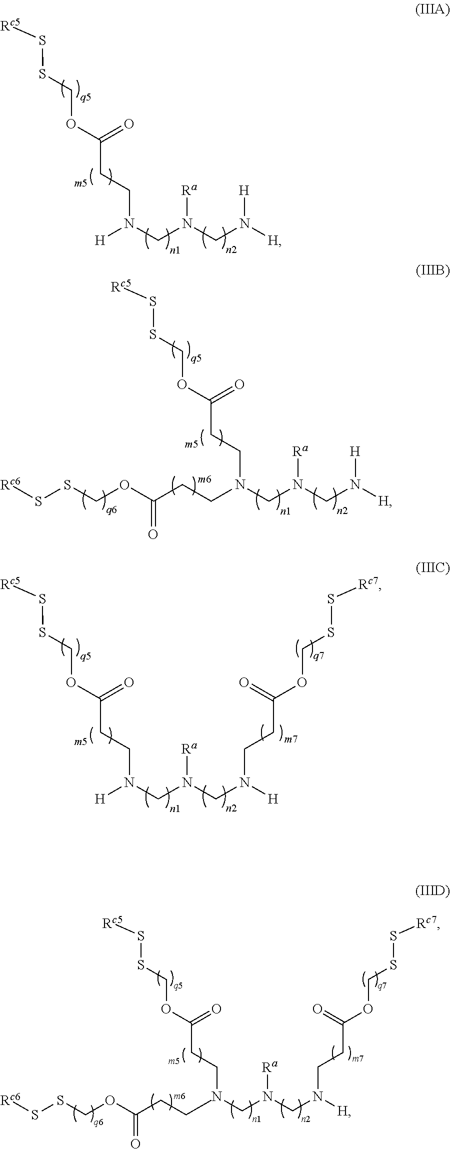

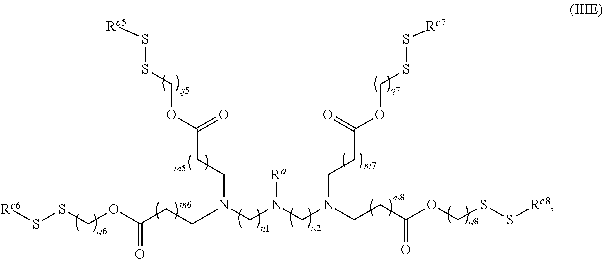

or a pharmaceutically acceptable salt thereof, wherein:
m5, m6, m7, and m8, are each independently 1, 2, or 3;
q5, q6, q7 and q8, are each independently 1, 2, 3, or 4; and
R^{c5}, R^{c6}, R^{c7} and R^{c8}, when present, are each independently C₄-C₂₀ alkyl or C₄-C₂₀ alkenyl.

Embodiment 120. The composition of embodiment 119, wherein the lipidoid of Formula (I), or the pharmaceutically acceptable salt thereof, is the lipidoid of Formula (III_{E}) or the pharmaceutically acceptable salt thereof.

Embodiment 121. The composition of embodiment 119 or 120, wherein R^{c5} is C₄-C₁₆ (e.g., C₆-C₁₂) alkyl or C₄-C₁₆ (e.g., C₆-C₁₂) alkenyl.

Embodiment 122. The composition of any one of embodiments 119-121, wherein, in Formula (III_{B}), (III_{D}), or (III_{E}), R^{c6} is C₄-C₁₆ (e.g., C₆-C₁₂) alkyl or C₄-C₁₆ (e.g., C₆-C₁₂) alkenyl.

Embodiment 123. The composition of any one of embodiments 119-122, wherein, in Formula (III_{B}), (III_{D}), or (III_{E}), R^{c6} is C₆-C₁₂ alkyl or C₆-C₁₂ alkenyl.

Embodiment 124. The composition of any one of embodiments 119-123, wherein, in Formula (III_{C}), (III_{D}), or (III_{E}), R^{c7} is C₄-C₁₆ (e.g., C₆-C₁₂) alkyl or C₄-C₁₆ (e.g., C₆-C₁₂) alkenyl.

Embodiment 125. The composition of any one of embodiments 119-124, wherein, in Formula (III_{C}), (III_{D}), or (III_{E}), R^{c7} is C₆-C₁₂ alkyl or C₆-C₁₂ alkenyl.

Embodiment 126. The composition of any one of embodiments 119-125, wherein, in Formula (III_{E}), R^{c8} is C₄-C₁₆ (e.g., C₆-C₁₂) alkyl or C₄-C₁₆ (e.g., C₆-C₁₂) alkenyl.

Embodiment 127. The composition of any one of embodiments 119-126, wherein, in Formula (III_{E}), R^{c8} is C₆-C₁₂ alkyl or C₆-C₁₂ alkenyl.

Embodiment 128. The composition of any one of embodiments 119-127, wherein m5 is 1 or 2.

Embodiment 129. The composition of any one of embodiments 119-128, wherein, in Formula (III_{B}), (III_{D}), or (III_{E}), m6 is 1 or 2.

Embodiment 130. The composition of any one of embodiments 119-129, wherein, in Formula (III_{C}), (III_{D}), or (III_{E}), m7 is 1 or 2.

Embodiment 131. The composition of any one of embodiments 119-130, wherein, in Formula (III_{E}), m8 is 1 or 2.

Embodiment 132. The composition of any one of embodiments 119-131, wherein q5 is 1, 2, or 3.

Embodiment 133. The composition of any one of embodiments 119-132, wherein, in Formula (III_{B}), (III_{D}), or (III_{E}), q6 is 1, 2, or 3.

Embodiment 134. The composition of any one of embodiments 119-133, wherein, in Formula (III_{C}), (III_{D}), or (III_{E}), q7 is 1, 2, or 3.

Embodiment 135. The composition of any one of embodiments 119-134, wherein, in Formula (III_{E}), q8 is 1, 2, or 3.

Embodiment 136. The composition of embodiment 115, wherein the lipidoid is selected from:

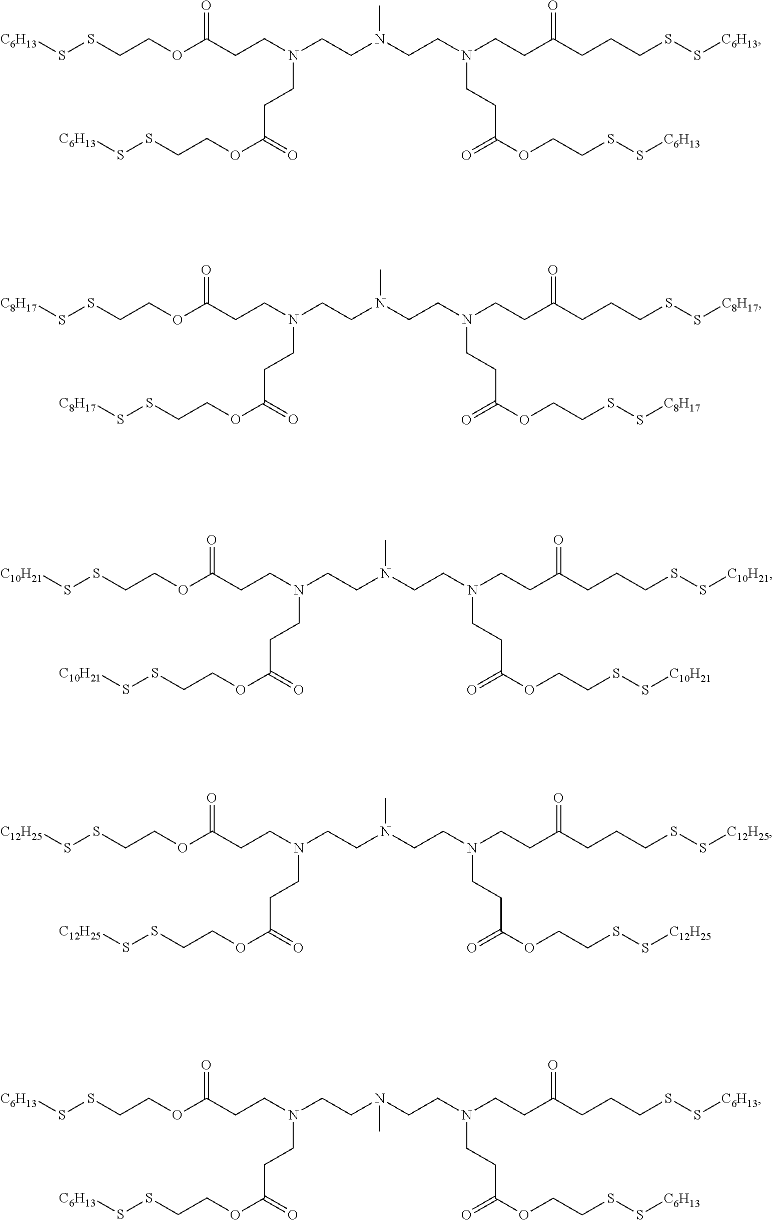

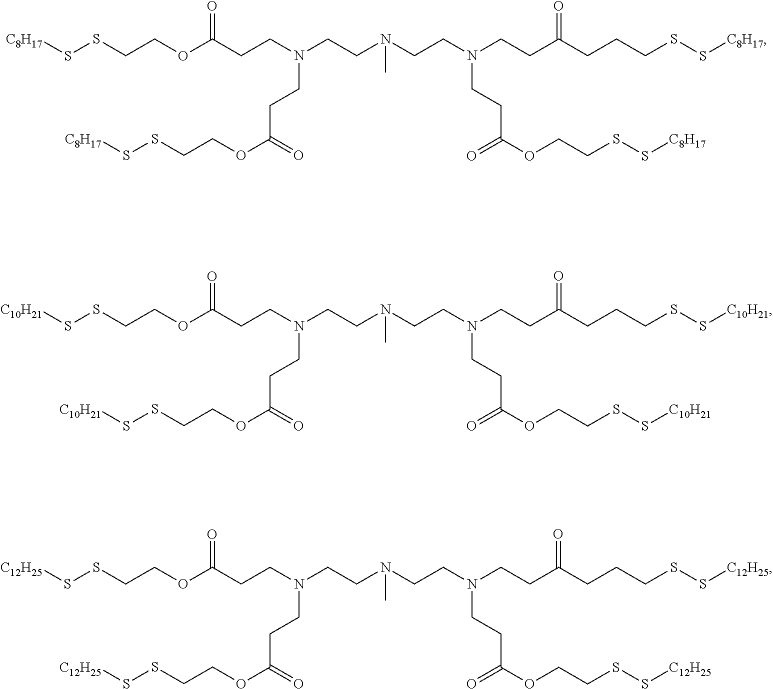

and any pharmaceutically acceptable salt of any of the foregoing.

Embodiment 137. The composition of any one of embodiments 115-136, wherein the lipidoid of Formula (I), (III_{A}), (III_{B}), (III_{C}), (III_{D}), or (III_{E}), or the pharmaceutically acceptable salt thereof, is present in the lipid composition at a molar percentage of about 20% to about 50%.

Embodiment 138. The composition of any one of embodiments 115-137, wherein the lipid composition comprises a steroid or steroid derivative at a molar percentage of about 10% to about 50%.

Embodiment 139. The composition of any one of embodiments 115-138, wherein the lipidoid of Formula (I), (III_{A}), (III_{B}), (III_{C}), (II_{D}), or (III_{E}), or the pharmaceutically acceptable salt thereof, is present in the composition at a mass or weight ratio to the pharmaceutical agent of about 5:1 to about 100:1.

Embodiment 140. The composition of any one of embodiments 115-139, wherein the composition is for a preferential delivery of the pharmaceutical agent to liver or a liver cell as compared to a delivery to a non-liver organ or a non-liver cell.

Embodiment 141. The composition of embodiment 140, wherein the non-liver organ is a lung, and wherein the non-liver cell is a lung cell.

Embodiment 142. The composition of embodiment 140 or 141, wherein the pharmaceutical agent encodes or is configured to up-regulate a target gene or a gene product thereof that is specific to or primarily found in the liver or the liver cell.

Embodiment 143. The composition of embodiment 140 or 141, wherein the pharmaceutical agent encodes or is configured to down-regulate a target gene or a gene product thereof that is specific to or primarily found in the liver or the liver cell.

Embodiment 144. The composition of any one of embodiments 140-143, wherein the target gene is an angiopoietin-like 3 (ANGPTL3) gene.

Embodiment 145. The composition of embodiment 142 or 143, wherein the pharmaceutical agent is configured to provide a modified expression profile of the target gene or the gene product thereof (e.g., the target protein or the functional variant thereof, or the target transcript) in the liver or the liver cell.

Embodiment 146. The composition of any one of embodiments 140-145, wherein the pharmaceutical agent is associated with a liver disease or disorder.

Embodiment 147. A method for preferential delivery of a pharmaceutical agent to liver or a liver cell in a subject in need thereof, the method comprising administering the composition according to any one of embodiments 115-146, thereby providing a greater amount (e.g., at least about 2-fold greater amount) of expression or activity of the pharmaceutical agent in the liver or the liver cell of the subject as compared to that achieved in a non-liver organ or a non-liver cell in the subject.

Embodiment 148. A method for preferential delivery of a pharmaceutical agent to liver or a liver cell in a subject in need thereof, the method comprising administering the composition according to any one of embodiments 115-146, thereby providing a at least about 5-fold greater amount, expression or activity of the pharmaceutical agent in the liver or the liver cell of the subject as compared to that achieved in a non-liver organ or a non-liver cell in the subject.

Embodiment 149. A method for preferential delivery of a pharmaceutical agent to liver or a liver cell in a subject in need thereof, the method comprising administering the composition according to any one of embodiments 115-146, thereby providing a at least about 10-fold greater amount, expression or activity of the pharmaceutical agent in the liver or the liver cell of the subject as compared to that achieved in a non-liver organ or a non-liver cell in the subject.

Embodiment 150. The method of any one of embodiments 147-149, wherein the non-liver organ is a lung, and wherein the non-liver cell is a lung cell.

Embodiment 151. A method for preferential delivery of a pharmaceutical agent to liver or a liver cell in a subject in need thereof, the method comprising administering the composition according to any one of embodiments 115-150, thereby providing a greater amount (e.g., at least about 2-fold greater amount) of expression or activity of the pharmaceutical agent in the liver or the liver cell of the subject as compared to that achieved with a corresponding reference lipid composition comprising a corresponding reference lipidoid.

Embodiment 152. A method for preferential delivery of a pharmaceutical agent to liver or a liver cell in a subject in need thereof, the method comprising administering the composition according to any one of embodiments 115-150, thereby providing a at least about 5-fold greater amount, expression or activity of the pharmaceutical agent in the liver or the liver cell of the subject as compared to that achieved with a corresponding reference lipid composition comprising a corresponding reference lipidoid.

Embodiment 153. A method for preferential delivery of a pharmaceutical agent to liver or a liver cell in a subject in need thereof, the method comprising administering the composition according to any one of embodiments 115-150, thereby providing a at least about 10-fold greater amount, expression or activity of the pharmaceutical agent in the liver or the liver cell of the subject as compared to that achieved with a corresponding reference lipid composition comprising a corresponding reference lipidoid.

Embodiment 154. The method of any one of embodiments 151-153, wherein the corresponding reference lipidoid has an amide-containing tail.

Embodiment 155. The method of embodiment 154, wherein the amide-containing tail has a corresponding lipidoid having structural Formula (II_{A}), (II_{B}), (II_{C}), (II_{D}), or (II_{E}).

Embodiment 156. The method of any one of embodiments 147 or 155, wherein the method modulates a greater amount or activity of a target gene or a gene product thereof in the liver or the liver cell of the subject as compared to that achieved with a corresponding reference lipid composition comprising a corresponding reference lipidoid.

Embodiment 157. The method of embodiment 156, wherein the target gene is an angiopoietin-like 3 (ANGPTL3) gene.

Embodiment 158. The method of embodiment 156 or 157, wherein the corresponding reference lipidoid as an amide-containing tail.

Embodiment 159. The method of embodiment 158, wherein the amide-containing tail has a corresponding lipidoid having structural Formula (II_{A}), (II_{B}), (II_{C}), (II_{D}), or (II_{E}).

Embodiment 160. The method of any one of embodiments 156-159, wherein the method provides a modified expression profile of the target gene or the gene product thereof in the liver or the liver cell of the subject.

Embodiment 161. The method of any one of embodiments 1-160, wherein the gene product is a target protein or a functional variant thereof the target transcript.

### EXAMPLES

In order that the invention described herein may be more fully understood, the following examples are set forth. The examples described in this application are offered to illustrate the compounds, compositions, materials, device, and methods provided herein and are not to be construed in any way as limiting their scope.

### Materials and Methods

### Study Design:

The overall objective of this study is to explore a non-viral lipid nanoparticle (LNP)-mediated CRISPR-Cas9 mRNA-based strategy for in vivo organ-specific genome editing of Angptl3 for blood lipids regulation. The study comprises (i) identifying a liver-targeted bioreducible lipid nanoparticle platform that can effectively deliver mRNA to hepatocytes; (ii) optimizing the LNP formulation to maximize the in vivo mRNA delivery efficacy; (iii) demonstrating in vivo genome editing effects of 306-O12B LNP-mediated Cas9 mRNA delivery by targeting the therapeutically relevant Angptl3 gene, resulting in profound reductions in serum ANGPTL3, low density lipoprotein cholesterol, and triglyceride levels; and (iv) revealing that the therapeutic effect of LNP-mediated CRISPR-Cas9 mRNA-based genome editing of Angptl3 was stable for at least one hundred days after a single dose administration. All animal experiments were conducted according to the protocols approved by the Tufts University Institutional Animal Care and Use Committee (IACUC) and performed in accordance with the National Institutes of Health (NIH) guidelines for the care and use of experimental animals. The genome editing efficacy, off-target side effects, safety profile, and changes in levels of lipids were thoroughly evaluated.

### LNPs Formulation:

The lipidoids were synthesized as per previous reports (S. Tenzer et al., Rapid formation of plasma protein corona critically affects nanoparticle pathophysiology. Nat Nanotechnol 8, 772-781 (2013), K. A. Hajj et al., Branched-Tail Lipid Nanoparticles Potently Deliver mRNA In Vivo due to Enhanced Ionization at Endosomal pH. Small 15, e1805097 (2019)). LNPs were prepared using a NanoAssemblr microfluidic system (Precision Nanosystems). Briefly, lipidoids, cholesterol (Sigma), phospholipids (DSPC, DOPE, and DOPC, Avanti Polar Lipids), and methoxypolyethylene glycol (DMG-PEG₂₀₀₀) (Avanti Polar Lipids) were dissolved in 100% ethanol at molar ratios of 50/38.5/10/1.5 at a final lipidoids concentration of 10 mg/mL. The MC-3 LNP formulation was prepared by following the procedure as previously described by Sedic et al with a slight modification. In brief, the lipids (6Z,9Z,28Z,31Z)-heptatriaconta-6,9,28,31-tetraen-19-yi 4-(dimethylamino)butanoate (MC-3), DSPC, Cholesterol, and DMG-PEG₂₀₀₀ were dissolved in pure ethanol at a molar ratio of 50% MC-3, 38.5% Cholesterol, 10% DSPC, and 1.5% DMG-PEG₂₀₀₀ with a final MC-3 concentration of 10 mg/mL. The lipid solution was then mixed with an acidic sodium acetate buffer containing mRNA (0.45 mg/mL, pH 4.0) by using the NanoAssemblr microfluidic system. The resulting LNP was dialyzed against PBS (pH 7.4, 10 mM) overnight at 4° C. Cas9 mRNA and gRNA (either sgANGPTL3 or sgLoxP) were mixed at the appropriate weight ratio in sodium acetate buffer (25 mM, pH 5.2). The mRNA solution and the lipid solution were each injected into the NanoAssemblr microfluidic device at a ratio of 3:1, and the device resulted in the rapid mixing of the two components and thus the self-assembly of LNPs. Formulations were further dialyzed against PBS (10 mM, pH 7.4) in dialysis cassettes overnight at 4° C. The particle size of formulations was measured by dynamic light scattering (DLS) using a ZetaPALS DLS machine (Brookhaven Instruments). RNA encapsulation efficiency was characterized by Ribogreen assay.

### In Vivo LNPs Delivery

All procedures for animal experiment were approved by the Tufts University Institutional Animal Care and Use Committee (IACUC) and performed in accordance with the National Institutes of Health (NIH) guidelines for the care and use of experimental animals. All the animals were ordered from Charles River. Female Balb/c mice (6-8 weeks) were used for in vivo firefly luciferase mRNA (fLuc mRNA, TriLink Biotechnologies) encapsulated LNPs screening and formulations optimization. Briefly, fLuc mRNA LNPs were intravenously injected into the mice at a dose of 0.5 mg/kg mRNA. At predetermined time point, mice were injected with 100 μL of D-Luciferin potassium salt (Goldbio) solution (15 mg/mL in PBS), anesthetized under isoflurane anesthesia, and measured by IVIS imaging system (Caliper Life Sciences).

### Commercially-Sourced Nucleic Acids

Cas9 mRNA was sourced from Tri-Link Biotechnologies. All sgRNAs used in this manuscript were sourced from Synthego, using their “end-modified” synthesis. Briefly, the first and last three bases of these gRNA are synthesized using 2′-O-Methyl-nucleosides, joined via 3′ phosphorothioate bonds. All other bases throughout the rest of the molecule are traditional ribonucleosides and phosphate bonds that are typical for native RNA.

In Vivo Cas9 mRNA/sgLoxP Delivery

Cas9 mRNA (TriLink Biotechnologies) and LoxP-targeted single guide RNA (sgLoxP, sequence: 5′-AAGTAAAACCTCTACAAATG, end-modified, Synthego) were coloaded into 306-O12B LNPs and intravenously injected into female Ai14 mouse at a dose of 1.65 mg/kg total RNA. At day 7 post-injection, mouse organs were collected and imaged by IVIS to detect the tdTomato expression. The liver tissue was further sectioned.

### Immunostaining

Tissue samples were embedded with OCT, frozen completely in liquid nitrogen, and stored at −80° C. until ready for sectioning. The frozen tissue block was sectioned into a desired thickness (10 μm) using the cryotome and placed onto glass slides suitable for immunofluorescence staining. The tissue sections were fixed with pre-cooled acetone (−20° C.) for 10 min, and then washed twice with PBS, 5 min each. The fixed tissue sections were incubated in 10% BSA blocking buffer at room temperature (r.t.) for 1 h, and then washed with PBS. A hepatocyte specific primary antibody (1:100 diluted in 1% BSA buffer, anti-hepatocyte specific antigen (HepPar1), Novus) (43) was applied to the sections on the slides and incubated in a humidified chamber at 4° C. overnight. The slices were rinsed with PBS for 2 changes, 5 min each, and then stained with eFlour660 conjugated F(ab′)2-Goat anti-mouse secondary antibody (1:50, Invitrogen) and incubated in a humidified chamber protected from light at room temperature for 1 h, and then washed 3 times with PBS. Fluorescent mounting medium containing DAPI(Sigma) was used to coverslip the slides. Sections were analyzed using a Leica SP8 confocal microscope.

### In Vivo Genome Editing of Angptl3:

Guide RNA sequence targeting Angptl3 was designed using the Benchling software. Female wild-type C57BL/6 mice, aged 6-8 weeks, were intravenously dosed with Cas9 mRNA and ANGPTL3-targeted single guide RNA (sgAngptl3, sequence: 5′-AGCCCTTCAACACAAGGTCA, end-modified, Synthego) co-loaded 306-O12B LNP at a dose of 1.0, 2.0, and 3.0 mg/kg in total RNA. PBS administrated mice were treated as negative control. At day 7 post-injection, mice were sacrificed (without fasting), blood was collected for circulating ANGPTL3 protein and blood lipids quantitation by ELISA, and liver tissue was collected from the median and left lateral lobe for DNA extraction and next generation sequence (NGS) analysis. To control for the influence of circadian rhythm on serum lipid levels, serum was collected at approximately the same time of day (early afternoon) for all experiments. In addition, to evaluate the in vivo toxicity and immune response, blood was collected and proceeded to serum from mice 2 days after the injection. Aspartate aminotransferase (AST) and alanine aminotransferase (ALT), and tumor necrosis factor alpha (TNF-alpha) were measured using assay kits for AST (G-Biosciences), ALT (G-Biosciences), and TNF-alpha (R&D Systems) per manufacturer's protocols. Serum levels of IFN-α, IL-6, and IP-10 were determined with the mouse IFN-α ELISA Kit (PBL Assay Science), mouse IL-6 Quantikine ELISA Kit (R&D Systems), and Abcam mouse IP-10 ELISA Kit, respectively.

### NGS Sequencing Analysis:

DNA was extracted from the median and left lateral lobe of the liver using a commercial extraction kit (Qiagen DNEasy Blood & Tissue). PCR primers were designed to amplify the region surrounding the target site in the Angptl3 gene, or the regions surrounding the predicted off-target sites (Table 1). Off-target sites were predicted using the Cas-Off-Finder software (rgenome.net/cas-offinder/). PCR amplicons were prepared for sequencing on an Illumina MiSeq (Tufts Genomics Core Facility). Sequencing data was analyzed using the OutKnocker 2 software (outknocker.org/outknocker2.htm).

TABLE 1
PCR primers used in this study

| ID | Forward | Reverse |
|---|---|---|
| Angptl3 | CTCCAAAGCCCTGACCTTGT | TCTGCACCTTCAGAGCCAAA |
| NGS | | |
| Angptl3 | TTCTGCACCTTCAGAGCCAA | GCAAAGCAAACCCTGAACTGA |
| T7E1 | | |
| Off1 | ACCTTGGTTAGGATGCCTGC | TTGCATTCCTGTCAGAGTGAT |
| Off2 | ACATAGCCATCCCCCAACAA | GCTGCAACTTGGTGCTACCT |
| Off3 | TGACATGACTCATTGCCACCA | GTGAACCATATCTTAATAGCACCAT |
| Off4 | TCTAAATGCCAGGGTTCTGACT | CATGCCATGTGGGGTGATACAA |
| Off5 | TACCCAGCCATATTGTGCAG | AGAAGACCAGATAGAAGTCAGGATG |
| Off6 | CGGATGTTTGCATGAGGGGA | GGTTAGCTGCTGGACACTGA |
| Off7 | GGTGTCCACTTGTTAAATGTCA | ACAACAACATGGAAAACTCTA |
| Off8 | GCCAGCCACAGTTTTCATCG | GGGAGTCACAGTCATGGGTC |
| Off9 | GCAGTCATCAGACAGGAGGG | GCAGCTCTCCAATCCAGACA |

### Serum ANGPTL3 Protein, Low Density Lipoprotein Cholesterol (LDL-C), and Triglyceride (TG) Analysis

Mouse blood was collected without using an anticoagulant and allows to clot for 2 h at r.t., and centrifuged at 2000×g for 15-20 min at r.t. to collect mouse serum. Serum levels of ANGPTL3 protein, LDL-C, and TG were determined using a Mouse Angiopoietin-like 3 Quantikine ELISA kit (R&D systems), Mouse LDL-Cholesterol kit (Crystal Chem), and Triglyceride Colorimetric Assay kit (Cayman Chemical) as per manufacturer's protocols, respectively.

### T7E1 Cleavage Assay:

The genomic regions flanking the on-target sites were amplified using extracted genomic DNA template, Platinum SuperFi Green DNA polymerase (Invitrogen), and specific primers (see Table 1). The following cycles were run: 30 s at 98° C., followed by 33 cycles of 10 s at 98° C., 15 s at 65° C., and 30 s at 72° C., followed 10 min at 72° C. The PCR products were purified using the GeneJET PCR Purification Kit (Thermo Scientific). 400 ng of purified PCR products were hybridized in NEBuffer 2 (New England Biolabs) by heating to 95° C. for 5 min, followed by a 2° C./second ramp down to 85° C. and a 0.1° C./second ramp down to 25° C. on a AppliedBiosystems PCR system (Thermo Fisher Scientific). The annealed samples were digested by T7 Endonuclease I (New England Biolabs) at 37° C. for 15 min, followed by incubating at 65° C. for 5 min to stop the reaction. The products were further purified and run on a 4-20% Novex TBE gel (Invitrogen).

### Statistical Analysis:

Data were expressed as mean±SD. All data were analyzed using Graphpad Prism software. The statistical difference was analyzed by One-Way ANOVA. *p<0.05 was considered significant, and **p<0.01, ***p<0.001 were considered highly significant.

### Lipidoid Nanoparticles Synthesis.

Lipidoids were synthesized through the Michael addition reaction between amine heads and alkyl-acrylate tails as previous reports. Lipidoid nanoparticles were formulated by rapidly mixing active lipidoids, cholesterol (Sigma Aldrich), DOPC (Avanti Polar Lipids), and mPEG₂₀₀₀-DMG (Avanti Polar Lipids) at a molar ratio of 50:38.5:10:1.5 in ethanol solution with sodium acetate buffer (pH 5.2, 25 mM) containing mRNA by using the NanoAssemblr microfluidic system. The final weight ration of active lipidoid to mRNA was set at 10/1. The resulting LNPs were further dialyzed against PBS (10 mM, pH 7.4) in 3,500 MWCO cassettes (Thermo Scientific) overnight at 4° C. The size and surface zeta potential of LNPs were measured by using a ZetaPALS DLS machine (Brookhaven Instruments).

In Vivo mRNA Delivery

All animal experiments were conducted in accordance with the approved animal protocols. For in vivo firefly luciferase mRNA (fLuc mRNA) transfection, female Balb/c mice aged at 6-8 weeks purchased from Charles River were intravenously (i.v.) administered with fLuc mRNA loaded LNPs (0.5 mg/kg mRNA). At 6 h post-injection, mice were intraperitoneally injected with D-Luciferin (Goldbio, 15 mg/mL in PBS) and imaged by using the IVIS imaging system (Perkin Elmer). To further quantify the luciferase protein expression in different organs, tissues were harvested, homogenized, and centrifuged to collect the supernatant. The supernatant was then assayed for luciferase expression using Firefly Luciferase Assay Kit 2.0 (Biotium). The results were presented as nanogram luciferase protein per gram of tissue.

For gene recombinant Cre mRNA delivery, LNPs co-formulated with Cre mRNA were i.v. injected into Ai14 Cre reporter mice (The Jackson Laboratory). After 7 d, mice were sacrificed, and major organs were collected and imaged using an IVIS imaging system. To further identify the specific cell populations that were transfected, tissues were frozen sectioned into 10 μm in depth, and further imaged by using an SP8 confocal microscope (Leica). For flow cytometry studies, lungs were minced and then treated with a mouse Lung Dissociation Kit (Milteny Biotec). Next, the lung solution was filtered through a 70-μm strainer to proceed to single-cell suspension, centrifuged, and then treated with red blood cell lysis buffer (eBioscience) for 5 min. The solution was then centrifuged again to harvest a cell pellet. Cells were resuspended in flow cytometry staining buffer (eBioscience), and then antibodies were added and incubated for 30 min on ice in the dark. The stained cells were washed twice with cold PBS, resuspended in PBS, and measured using an LSR-II flow cytometer (BD Bioscience). The antibodies used in this study are as follows: CD326-PE-Cy7 (eBioscience), CD31-FITC (Biolegend), and F4/80-eFluor 660 (eBioscience). The dilution rates of all antibodies were used as per manufacturers' suggestions.

For genome editing Cas9 mRNA delivery, Cas9 mRNA-loaded LNPs were i.v. injected into Balb/c mice (0.5 mg/kg). 6 h post-injection, mice were sacrificed, and organs were collected, and western blot was performed to detect the Cas9 expression in different organs. Briefly, organs were homogenized in lysis buffer and centrifuged at 13,000 g for 10 min at 4° C. The supernatants were collected, and the protein concentrations were measured using a BCA assay kit (Thermo Fisher). Then 20 μg of total proteins were loaded onto a 4-20% polyacrylamide gel (Thermo Fisher) and the gel was then run for 90 min under a stable voltage of 120 V. The gel was cut and transferred to a PVDF membrane, blocked with 5% skimmed milk for 1 h at room temperature, and incubated with anti-CRISPR-Cas9 primary antibody (Abcam, ab189380) overnight at 4° C. The membrane was washed with TBST 5 times, incubated with HRP rabbit anti-mouse secondary antibody (Abcam) for 1 h at room temperature, and then imaged with an ECL substrate using a gel imaging system.

### Isolation of Protein Corona

Mouse plasma was centrifugated at 13, 000 g at 4° C. to remove protein aggregates before use. LNPs were mixed with an equal volume of C57BL/6 mouse plasma to mimic the protein concentration in vivo and incubated for 1 h at 37° C. under shaking. The protein corona-coated LNPs were isolated by centrifugation at 13,000 g for 30 min, followed by washing with cold PBS three times to remove unbound proteins. The same procedure was performed for plasma aliquots without adding the LNPs to verify the absence of protein precipitation. All experiments were conducted three times. The amount of proteins in protein corona coated LNPs was determined using a BCA assay kit (Thermo Fisher). The obtained protein samples were further lyophilized and stored at −20° C. for further experiment.

### Reduction, Alkylation, and Digestion of Proteins

The lyophilized protein samples were reconstituted to 1 mg/mL in M-PER Mammalian Protein Extraction Reagent (Thermo Scientific). For each sample, 100 μg of protein was reduced with 2.1 μL of 500 mM dithioerythritol (DTT), 99+% (Acros Organics) at 50° C. for 45-minutes. The samples were then alkylated with 11.5 ul of iodoacetamide (Acros Organics) in the dark for 30 min at room temperature. The samples were then digested with trypsin/Lys-C Mix (Mass Spec Grade, Promega) overnight at 37° C. at an enzyme-to protein ratio of 1-50. The samples were then stored at −20° C. until LC-MS analysis.

### Mass Spectrometry

The resulting samples were further analyzed by electrospray liquid chromatography mass spectrometry (LC-MS/MS) using an Orbitrap Fusion Lumos mass spectrometer (Thermo Fisher) coupled online to an EASY-nLC 1200 (Thermo Fisher). Samples were run at a flow rate of 300 nL/min using the following gradient: 0-4 minutes 0-5% B, 4-74 minutes 0-30% B, 74-79 minutes 30-90% B, 79-89 minutes 90% B, 89-94 minutes at 90-0% B, and 94-105 minutes 0% B for column re-equilibration. Mobile phase A was 96.1:3.9 0.1% FA in water/0.1% FA in ACN. Mobile phase B was 80.0:20.0 0.1% FA in water/0.1% FA in ACN. The samples were first desalted on a Thermo Fisher Scientific Acclaim PepMap 100 C18 HPLC column (3 μm particle size, 75 μm×2 cm, 100 Å) before separation on a Thermo Fisher Scientific PepMap RSLC C18 EASY-Spray Column (3 μm particle size, 75 μm×15 cm, 100 Å). The data was analyzed using the Proteome Discoverer 2.1.0.81 software (Thermo Fisher). The data was run against the Mus musculus Uniprot FASTA file (Modified 26 Aug. 2020) to identify the mouse proteins in the corona.

In Vitro mRNA Delivery

The TSC2-deficient TTJ cells were seeded in 48-well plates with 1×10⁴ cells per well. After 24 h incubation, cells were transfected with EGFP mRNA by using LNPs (100 ng of mRNA per well) for 24 h. Lipofectamine2k was used as a positive control. To measure the transfection efficiency, cells were digested with trypsin, collected, and further evaluated the GFP expression using flow cytometry (Attune NxT Cytometer, ThermoFisher). The percentage of EGFP-positive cells was calculated by Flowjo.

In Vivo Treatment of TTJ Tumor with TSC2 mRNA

C57BL/6 mice were tail vein injected with 2×10⁶ TSC2-deficient TTJ cells to form tumor nodules in the lungs. On day 24 after tumor cell inoculation, mice were randomly assigned to 3 groups: untreated control group, empty LNP treatment group, and Tsc2 mRNA-loaded LNP (dosage: 0.75 mg/kg of mRNA per injection) treatment group. Mice were treated every other day for a total of 5 times. After treatment, mice were euthanized, and lungs were collected for further morphological and immunohistochemical analyses.

Disclosed herein is a highly potent new non-viral LNP-mediated CRISPR-Cas9 delivery system for the liver delivery of Cas9 mRNA, and demonstrate its efficacy by targeting the Angptl3 gene. The system is composed of a leading tail-branched bioreducible lipidoid (306-O12B) co-formulated with an optimized mixture of excipient lipid molecules, and it successfully co-delivers SpCas9 mRNA and a single guide RNA targeting Angptl3 (sgAngptl3) via a single administration (FIG. 1). 306-O12B LNP specifically delivered Cas9 mRNA and sgAngptl3 to liver hepatocytes of wild-type C57BL/6 mice, resulting in a median editing rate of 38.5% and a corresponding 65.2% reduction of serum ANGPTL3 protein. The delivery using 306-O12B LNP was more efficient than delivery with MC-3 LNP, a gold standard LNP that was recently FDA-approved for liver-targeted delivery of nucleic acids. Moreover, liver specific knockdown of Angptl3 resulted in profound lowering of LDL-C and TG levels. Importantly, no evidence of off-target mutagenesis at 9 top predicted sites was observed, nor any apparent liver toxicity. The CRISPR-mediated genome editing was maintained at a therapeutically relevant level for at least one hundred days after the injection of a single dose. This system offers a clinically viable approach for liver-specific delivery of CRISPR-Cas9-based genome editing tools.

### Example 1: In Vivo Screening of Lipid Nanoparticles for mRNA Delivery

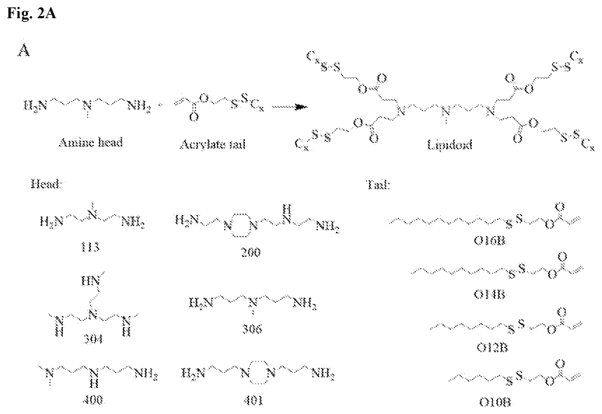

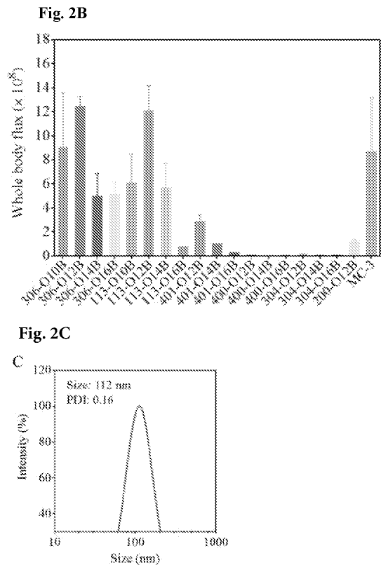

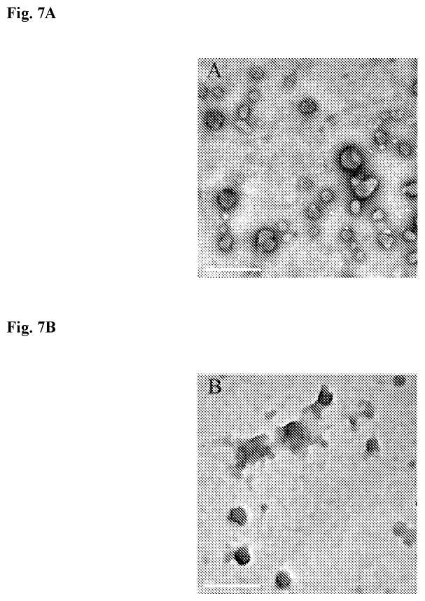

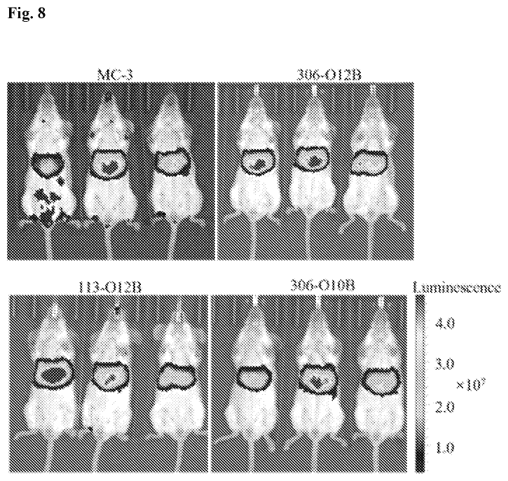

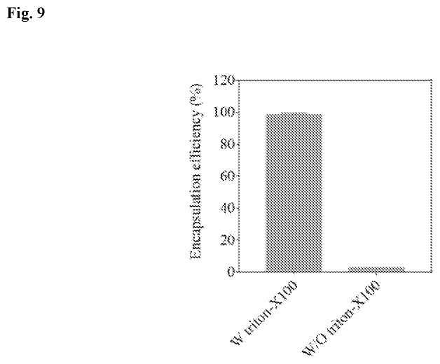

The tail-branched bioreducible lipidoids were prepared via a combinatory solvent free Michael-Addition reaction between disulfide bond-incorporated acrylate lipid tails and amine-containing heads according to previous reports (FIG. 2A). The in vivo mRNA delivery efficacy of these lipids was evaluated by encapsulating firefly luciferase mRNA (fLuc mRNA) into LNPs and delivering these LNPs intravenously to female wild-type Balb/c mice. These LNPs were formulated with disclosed synthetic ionizable lipids, along with the excipient compounds cholesterol, DSPC, and DMG-PEG. Representative transmission electron microscopy images of blank (unloaded) and fLuc mRNA-loaded LNPs are shown in FIG. 7A and FIG. 7B. The gold standard MC-3 LNP formulated with cholesterol, DSPC, and DMG-PEG was included as a positive control. Six hours after mRNA delivery, mice were injected intraperitoneally with luciferin substrate, and whole-body fLuc activity was measured using an IVIS in vivo imaging system (PerkinElmer). As shown in FIG. 2B, mRNA delivery with 306-O12B, 113-O12B, and 306-O10B LNPs resulted in comparable luciferase bioluminescence intensity as compared with MC-3 LNP delivery. In vivo images of mice showed that the luciferase protein was mainly expressed in the liver (FIG. 8). Based on these preliminary results, 306-O12B LNP was selected for further development due to its relatively high luciferase expression as well as the high specificity. The fLuc mRNA could be efficiently encapsulated into the 306-O12B LNP with an encapsulation efficiency of ˜96% (FIG. 9). Following the encapsulation of fLuc mRNA, 306-O12B LNPs had an average diameter of 112 nm (FIG. 2C)

### Example 2: Optimization of the Formulation of 306-O12B LNP

To further increase the luciferase expression in vivo, a variety of formulation parameters used for the assembly of these LNPs were optimized, including the excipient phospholipid identity, the molar composition ratios of the four-components of the LNP formulation, and the lipid/mRNA weight ratio of fLuc mRNA encapsulated 306-O12B LNP.

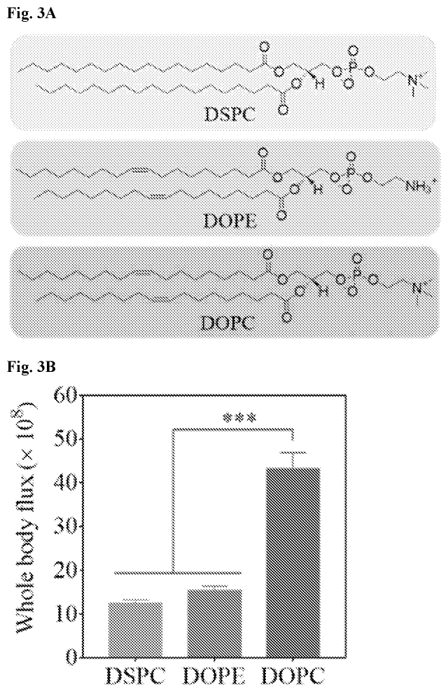

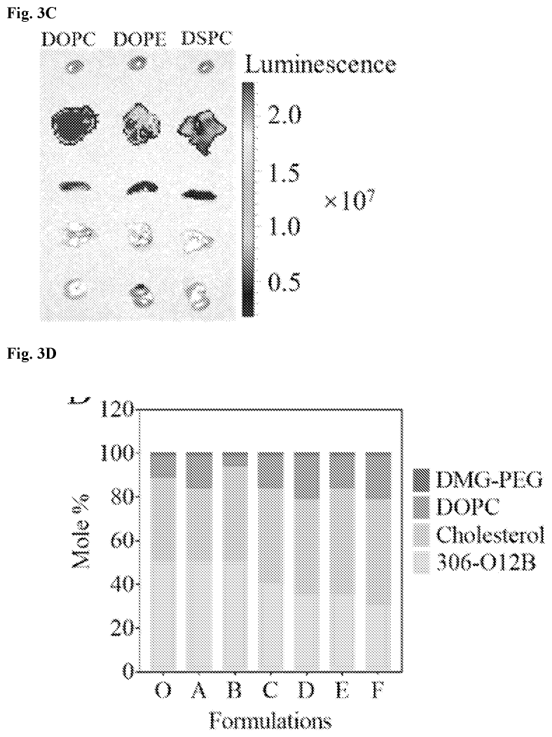

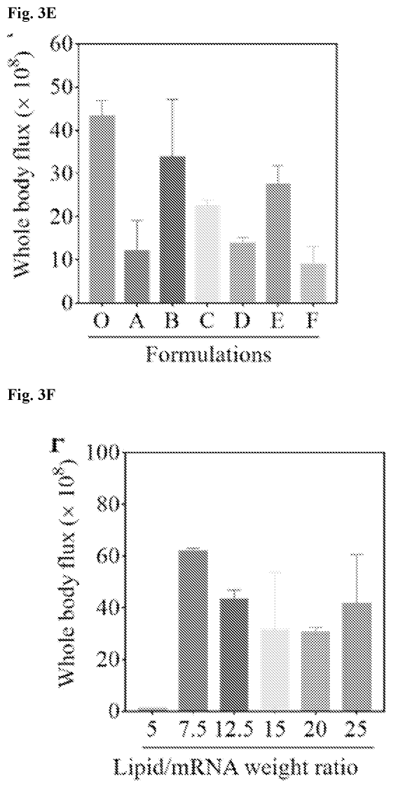

First, two additional phospholipids, DOPE and DOPC (FIG. 3A), which have similar structures but different head groups and tail saturation than the original DSPC phospholipid, were selected to evaluate the effect of phospholipid excipient on the efficacy of luciferase expression in vivo. DOPC and DOPE each contain one degree of unsaturation in the carbon tail, whereas DSPC is fully saturated. Furthermore, DSPC and DOPC each contain a quaternary amine headgroup, whereas DOPE contains a primary amine headgroup. Each of these features has been reported to influence LNP delivery efficiency in the literature. It has been reported that quaternized amine head groups show a stronger proton sponge effect than primary amine head groups, which can facilitate the endosomal escape of the cargo mRNA into the cytoplasm, thus increasing translation of the mRNA. Furthermore, the degree of saturation of the lipid tail has been shown to influence membrane fluidity, which may also influence endosomal escape. Unsaturated lipid tails may result in higher membrane fluidity, which may also help improve endosomal escape via destabilization of the endosomal membrane upon fusion of the LNP with the membrane. LNPs formed with DOPC, which contains a quaternary amine and unsaturated tail, would show the most efficient fLuc delivery. As shown in FIGS. 3B & 3C, fLuc mRNA LNPs formulated with DOPC indeed resulted in significantly higher luciferase expression in the liver than that of formulations with DOPE and the original DSPC phospholipid. fLuc mRNA delivered with DOPC-containing LNPs resulted in approximately 4-fold higher luminescence signal compared to the original DSPC-containing LNPs.

In initial screenings, the active lipid and excipient components were formulated at a molar ratio of [Lipid:Cholesterol:DSPC:DMG-PEG] of [50:38.5:10:1.5]. This excipient ratio has been reported in the literature to be effective. After identifying the optimal phospholipid as DOPC and adjusting the formulation accordingly, LNPs formulated at a variety of molar ratios were tested to identify the optimal parameters (FIG. 3D). As shown in FIG. 3E, the original formulation 0 (306-O12B:cholesterol:DOPC:DMG-PEG at a molar ratio of 50:38.5:10:1.5) displayed the highest luciferase bioluminescence intensity; none of the new formulation parameters surpassed the original.

In an effort to further increase the in vivo efficacy, the LNPs with this optimal ratio of four components were formulated with different weight ratios of active lipid 306-O12B:mRNA ranging from 5:1 to 25:1. Interestingly, the highest efficacy was achieved when the weight ratio was 7.5:1. It appears as though increasing the amount of lipid beyond this point does not improve in vivo delivery efficacy (FIG. 3F). Collectively, these results indicated that the optimized formulation of 306-O12B LNP had 50% 306-O12B, 38.5% cholesterol, 10% DOPC, and 1.5% DMG-PEG molar composition with a 7.5/1 weight ratio of 306-O12B/mRNA.

### Example 3: In Vivo Hepatocyte-Specific Codelivery of Cas9 mRNA and sgRNA with mRNA-Optimized 306-O12B LNP

Identification of the specific cell types that are preferentially edited by Cas9 mRNA/sgRNA LNPs is critical for evaluating the potential applications of a CRISPR delivery system. the Ai14 reporter mouse line was used, which is genetically engineered with a LoxP-Flanked STOP cassette controlling tdTomato expression. Although this mouse line is frequently used with Cre recombinase, successful CRISPR-mediated excision of the LoxP-flanked stop codon will also induce the expression of tdTomato. When paired with Cas9 and a guide RNA targeting this site, examination of the cells with tdTomato expression indicates which cell types LNP delivery system targets.

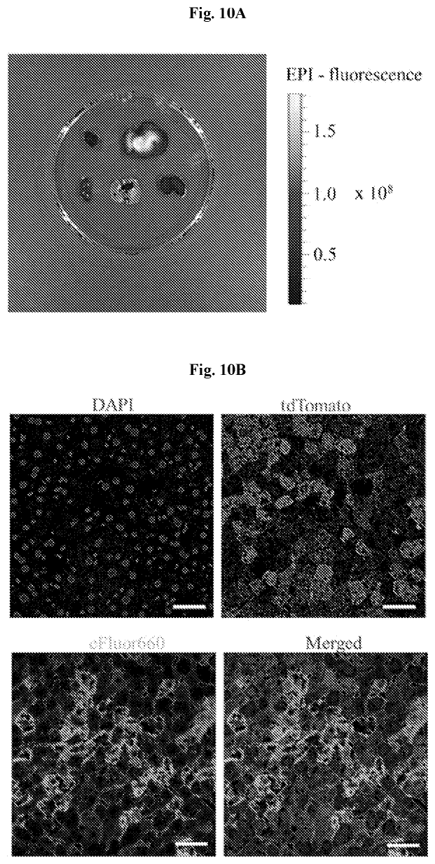

To validate this approach, the 306-O12B LNP was used to deliver the LoxP-targeted sgRNA (sgLoxP) to mice engineered to express both the Ai14 construct and a constitutively-expressed Cas9 construct (Ai14+/Cas9+ mouse model). FIGS. 10 A and 10B show 306-O12B LNP enabled sgLoxP-mediated genome editing in Ai14/Cas9 crossing mice. As shown in FIG. 10A, delivery of sgLoxP with optimized 306-O12B LNP system resulted in red fluorescence detected specifically in the liver. Interestingly, further histological analysis as shown in FIG. 10B revealed that the tdTomato signal was mainly observed in the liver hepatocytes, indicating that 306-O12B LNP can also specifically deliver sgRNA to this therapeutically relevant cell type.

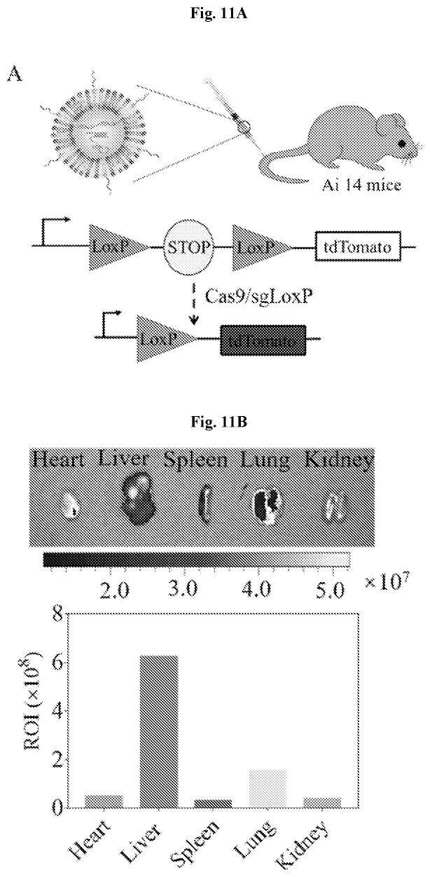

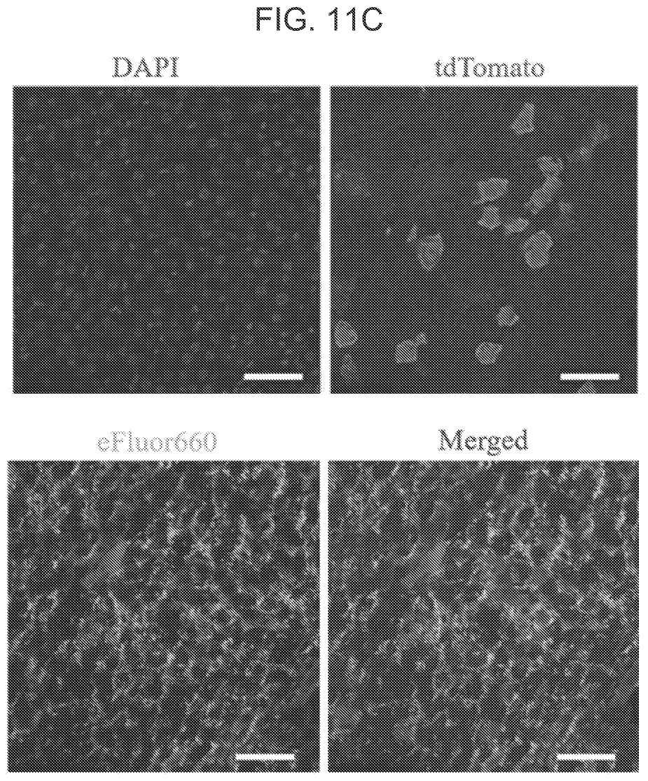

Next, Cas9 mRNA and sgLoxP were co-formulated into one single LNP and injected into Ai14 mice via tail vein at 1.65 mg/kg total RNA dose (FIG. 11A). Seven days after delivery, organs were harvested and imaged ex vivo using the IVIS system. Imaging of mouse organs showed that this system could indeed induce red fluorescence, indicating successful functional codelivery of both the mRNA and sgRNA components, and that tdTomato signal was predominantly detected in the liver (FIG. 11B). Immunofluorescent staining for hepatocyte specific antigen was also performed, and the confocal images demonstrated that the majority of the tdTomato protein was expressed in the hepatocytes (FIG. 11C). These findings indicate that 306-O12B LNP can specifically transport Cas9 mRNA and gRNA to liver hepatocytes.

### Example 4: In Vivo Genome Editing of Angptl3

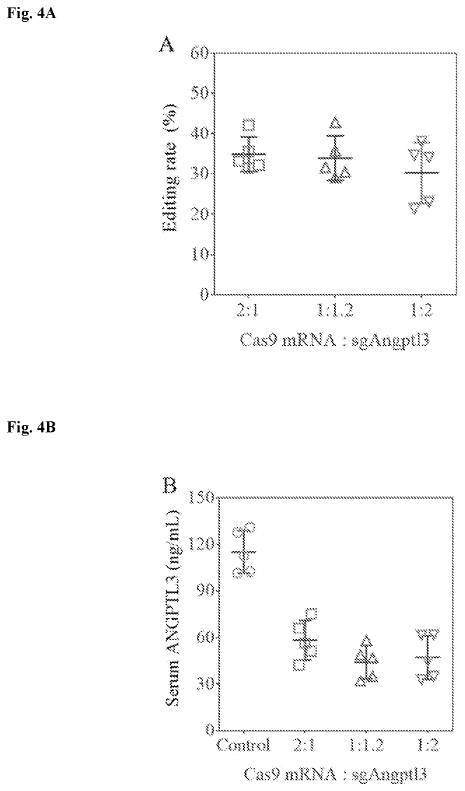

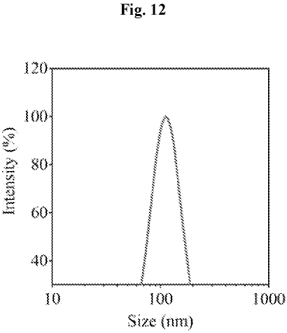

Encouraged by the positive results achieved above, the ability of 306-O12B LNP to deliver Cas9 mRNA to manipulate the expression of a functional endogenous gene was tested, Angptl3. Angptl3 encodes angiopoietin-like 3 (ANGPTL3), a central regulator of lipoprotein metabolism which inhibits both lipoprotein lipase and endothelial lipase activity. The ratio of Cas9 and guide RNA was explored, which may affect the in vivo genome editing efficacy, by injecting C57BL/6 mice with 306-O12B LNP co-formulated with different Cas9 mRNA to sgAngptl3 mass ratios of 2:1, 1:1.2, and 1:2 at a total RNA dose of 3.0 mg/kg. Nanoparticles were formulated using the optimized formulation parameters determined above; namely helper molecules at a molar ratio of 50:37.5:10:1.5 (306-O12B:cholesterol:DOPC:PEG), with a 306-O12B:total RNA weight ratio of 7.5:1. At day 7 after injection, blood serum was collected for ELISA analysis of serum ANGPTL3 protein levels, and liver tissue samples were collected for DNA extraction and next-generation sequencing (NGS) to determine targeted Cas9-mediated genome editing. Genome editing in liver tissue is shown in FIG. 4A, and a reduction of serum ANGPTL3 protein levels (FIG. 4B) at all Cas9 mRNA/sgAngptl3 ratios, however, no significant differences were observed between these groups. 1:1.2 Cas9 mRNA/sgAngptl3 ratio was used for the following experiments. The Cas9 mRNA/sgAngptl3 encapsulated 306-O12B LNP had similar characteristics as fLuc mRNA LNP with an average size of 110 nm (FIG. 12).

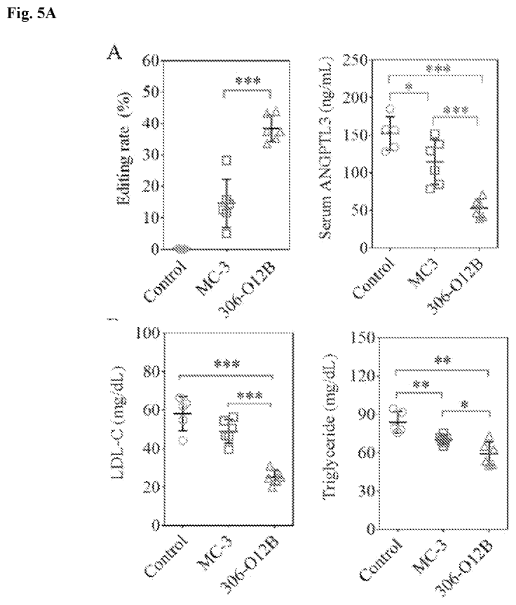

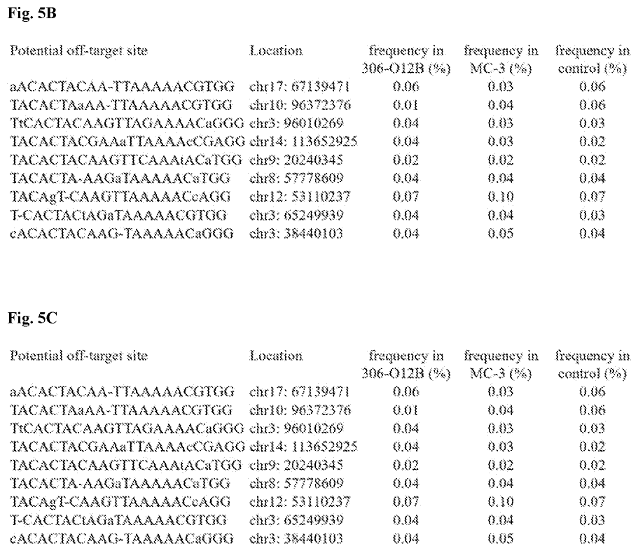

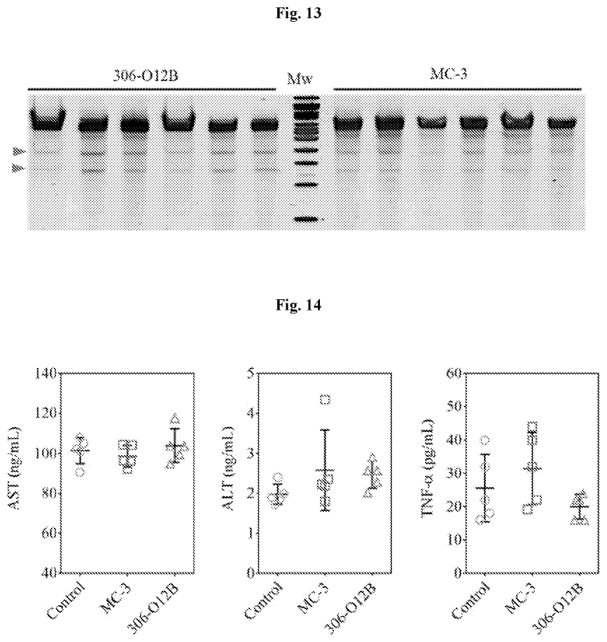

Given the significant levels of editing, a more detailed in vivo editing experiment was performed. As a gold standard, 306-O12B LNP was compared against LNP composed of the FDA-approved liver-targeting lipid MC-3 (termed MC3 LNP), encapsulating the same RNA components. Mice were administered with 306-O12B LNP or MC-3 LNP at a total RNA dose of 3.0 mg/kg. At day 7 after administration, the editing at the desired site in the liver using a T7E1 assay was observed (FIG. 13). NGS of the Angptl3 target site in liver samples further demonstrated that 306-O12B LNP-mediated delivery resulted in a median editing rate of 38.5%, which is significantly higher than that of MC-3 LNP mediated delivery (14.6%) (FIG. 5A). More importantly, the serum analyses revealed that the serum ANGPTL3 protein, LDL-C, and TG levels in 306-O12B LNP treatment group (65.2%, 56.8%, 29.4% reductions respectively) were significantly lower than that of MC-3 LNP treated mice (25%, 15.7%, 16.3% reductions respectively) (FIG. 5A). A detailed analysis of the NGS sequencing results revealed that the most frequent editing event was a 1-nt deletion precisely at the predicted Cas9 cut site, followed by a 1-nt insertion at the same location (FIG. 5B). As expected, the same editing events were observed in both MC3 LNP and 306-O12B LNP-treated livers, the main difference was in the frequency of these events. This indicates that the observed serum component reductions were likely a result of the Cas9-mediated genome editing, and implies that the difference in observed outcomes between the two lipids was solely a result of delivery efficiency, rather than a change in the innate activity of the Cas9 mRNA.

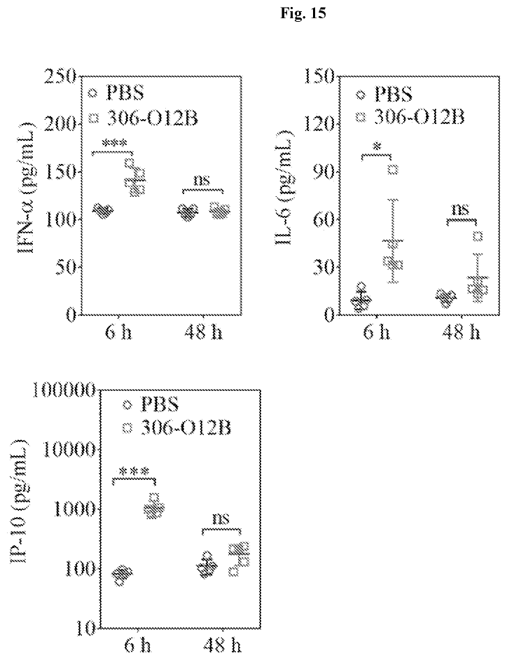

With any CRISPR delivery system, care must be taken to avoid off-target editing events or delivery-induced toxicity. The top 9 most likely off-target genomic mutagenesis sites were predicted computationally, and these loci were interrogated via NGS of DNA extracted from the liver. No evidence of editing at any of the 9-top predicted off-target mutagenesis sites was observed (FIG. 5C). To evaluate the in vivo toxicity and potential immunoinflammatory response, serum levels of the liver function markers aspartate aminotransferase (AST) and alanine aminotransferase (ALT) and of the proinflammatory cytokine tumor necrosis factor-alpha (TNF-alpha) were measured 48 h postdose. No significant changes in these markers were detected after treatment with the Cas9 LNP (FIG. 14). In addition, it was observed that the serum cytokine levels, i.e., IFN-α, IL-6, and IP-10 were upregulated at 6 h post administration. However, by 48 h post administration, the level of all cytokines returned to baseline, indicating that the delivery does not induce a long-term systemic inflammatory response (FIG. 15).”

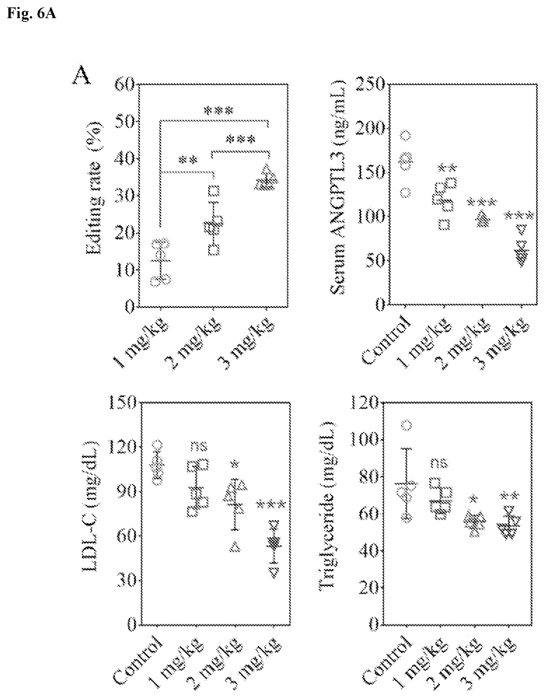

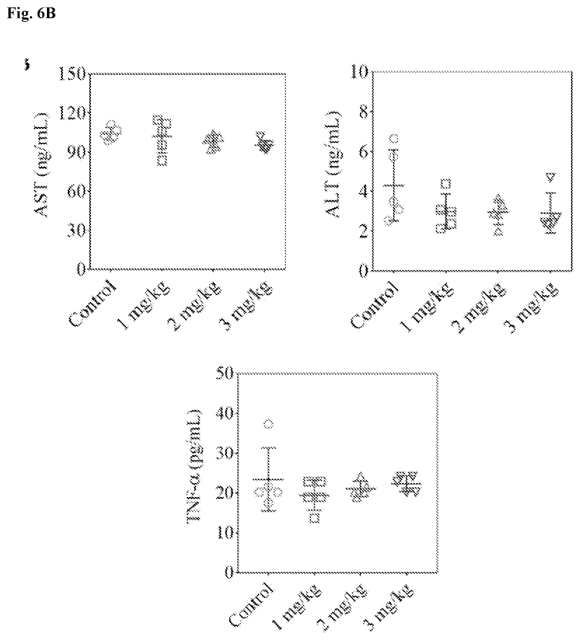

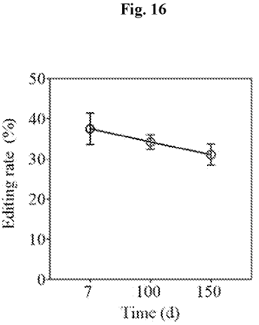

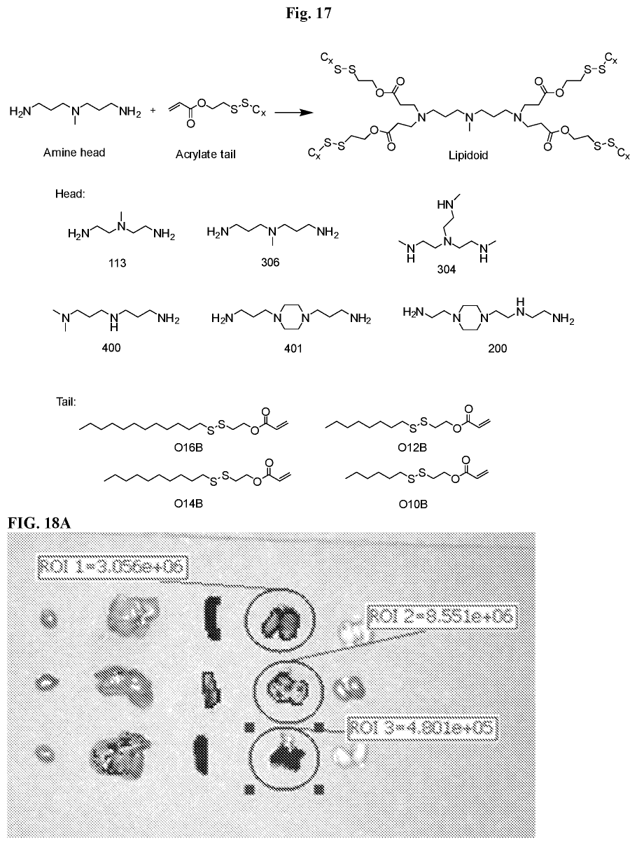

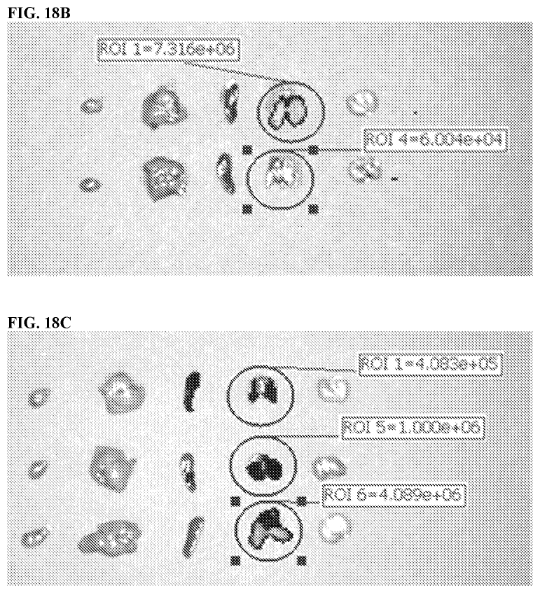

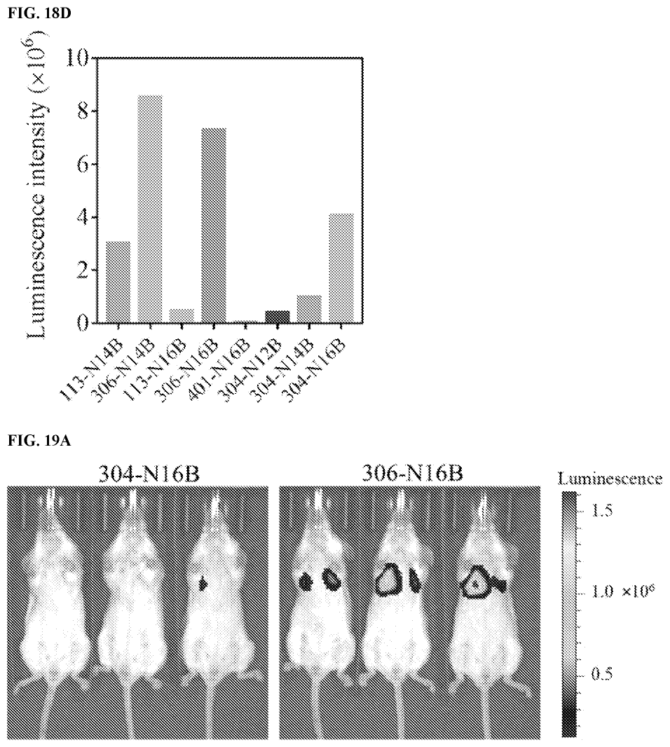

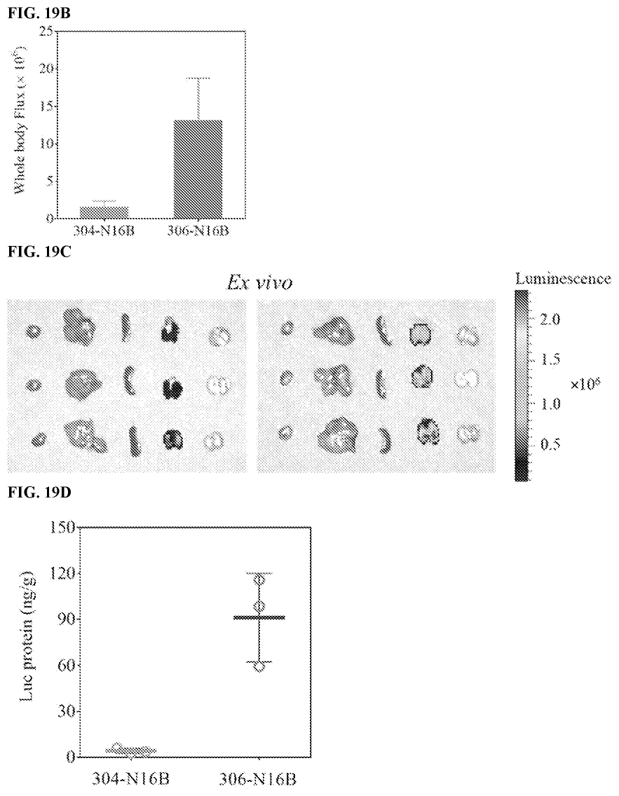

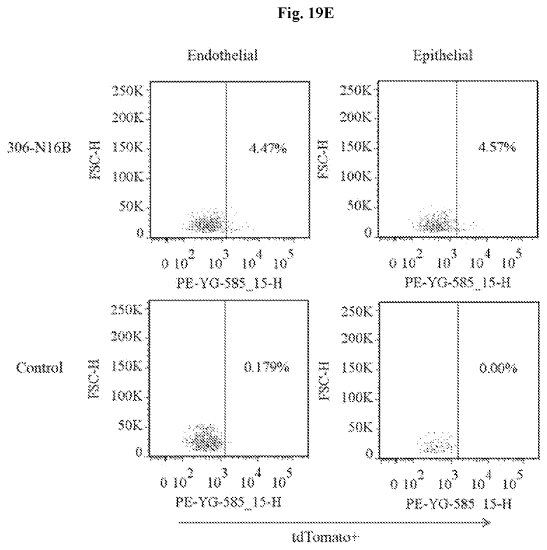

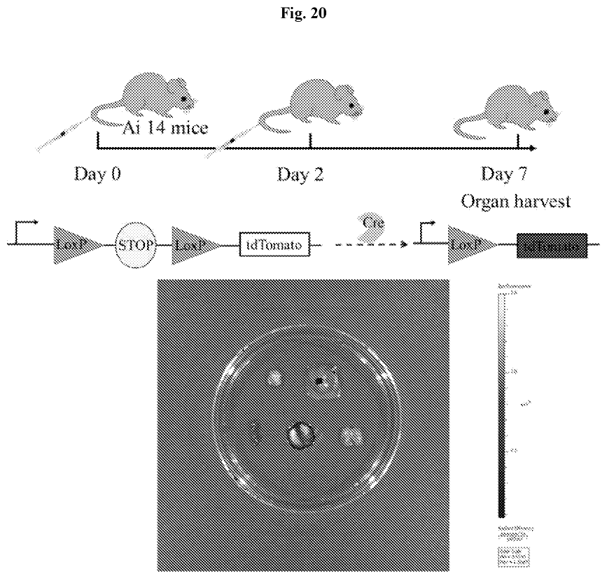

One of the tremendous advantages of CRISPR-Cas9 over other therapies is the potential to generate a long-term therapeutic efficacy with only one injection. To assess the long-term durability of editing and knockdown in the liver, 306-O12B LNP was injected into C57BL/6 mice at a total RNA dose of 1.0, 2.0, or 3.0 mg/kg. Importantly, it was found that the therapeutic effect was stable for at least one hundred days after a single dose administration. One hundred days after a single administration, a dose-dependent genome editing of Angptl3 in the liver and knockdown of serum ANGPTL3 was still observed, with profound reductions in serum ANGPTL3, LDL-C, and TG levels (60%, 48.1%, and 28.6% reductions respectively) at the highest dose (FIG. 6A). This indicates both the potential ability to titrate the knockdown effect by varying the total RNA dose, and the ability of a single dose to generate a therapeutically relevant knockdown effect over a longer time scale. No evidence of long-term systemic toxicity was observed (FIG. 6B). Furthermore, continued observation has indicated that the genomic editing could still be detected at least 150 days after the single initial administration of 306-O12B LNP (FIG. 16). While further study is necessary to establish the maximum duration of genomic editing effects after a single administration, as well as to confirm that the serum lipid profile continues to track with the genomic editing profile over longer time periods, this is a promising indicator of the long-term efficacy of delivery platform.

Taken together, these results indicated that 306-O12B LNP is more efficient than the gold standard MC-3 LNP in inducing loss-of-function mutations in Angptl3 through CRISPR-Cas9-based genome editing. The non-viral LNP mediated in vivo genome editing of Angptl3 in the liver led to therapeutically relevant reduction of blood lipid levels, with minimal off-target editing events, systemic toxicity, or inflammation in the mouse.

### Discussion

Disclosed herein is a highly potent lipidoid nanoparticle, composed of the synthetic, bioreducible lipidoid 306-O12B, capable of liver specific delivery of Cas9 mRNA and gRNA for in vivo Angptl3 editing. In recent years, the CRISPR-Cas9 system has emerged as a potent therapeutic platform to correct genetic disorders for disease treatment. However, the application of this technology has been significantly hampered for a number of reasons, many relating to the lack of safe and efficient delivery vehicles. First, the large size of the Cas9-gRNA complex makes it difficult to encode the entire complex within a single viral genome, or to deliver the 160 kDa protein directly into cells. Second, the host immune response to the CRISPR machinery or the delivery vector itself, as well as pre-existing anti-Cas9 antibodies or anti-AAV-antibodies in humans, can result in dramatically reduced CRISPR efficacy and potential immunotoxicity. Finally, the potential undesired host genome insertional mutagenesis of the CRISPR construct, or off-target genomic editing events caused by insufficient specificity of the CRISPR machinery, may induce fatal genotoxicity. Some of these issues can be ameliorated by delivering Cas9 as mRNA in complex with gRNA. For example, unlike plasmid DNA or virus, mRNA does not need to be transported into the cell nucleus; the translation of mRNA to protein occurs in the cytoplasm, thus simplifying the delivery path and avoiding the possibility of insertional mutagenesis of the CRISPR machinery. Furthermore, delivery of a mRNA compound results in transient expression of the CRISPR components, unlike viral delivery which may result in prolonged expression. It has been suggested that transient expression may reduce off-target genomic editing, simply by reducing the opportunity for such adverse editing events to occur.

To facilitate the clinical translation of mRNA-mediated CRISPR technology, delivery systems with superb safety, high specificity and efficacy are needed. Non-viral nanoparticles prepared from synthetic biomaterials have shown promise to improve cytosolic delivery of nucleic acids in vivo. Lipid nanoparticles (LNPs) are one of the most developed formulations for RNA delivery. In August 2018, the US Food and Drug Administration (FDA) approved the first LNPs-based siRNA formulation. Encouraged by the success of LNPs, tremendous efforts have been made using LNPs for in vivo mRNA transfection.

In vitro potency of an LNP formulation, however, rarely reflects its in vivo performance, and therefore, directly in vivo screening of a library of tail branched bioreducible lipidoids was conducted. It was found that the amine heads and the hydrophobic chain length in the lipidoids play critical roles in determining in vivo mRNA delivery efficacy. Lipid nanoparticle formulations are typically composed of an ionizable active lipid, a phosphor helper lipid, cholesterol, and lipid-anchored polyethylene glycol (PEG). The relative ratio of these components may have profound effects on the in vivo mRNA delivery efficacy. The published formulation conditions of MC-3 LNP as a starting formulation was used. While the structure of the lipidoid used is different from MC-3, the published LNPs use similar excipient lipids as those used for MC-3 LNP. The excipient lipid components may serve a similar role in both types of LNPs, and thus using similar excipient ratios would provide a valid starting point. To further maximize protein expression in vivo, these formulation parameters were optimized for the LNP used herein. The optimal formulation for the LNP contained 50% 306-O12B, 38.5% cholesterol, 10% DOPC, and 1.5% DMG-PEG (by molar ratio) with a 7.5/1 weight ratio of 306-O12B/mRNA.

Previous reports have shown that inhibition of ANGPTL3 activity by ANGPTL3 antibody or ASO provided therapeutically beneficial reduction in triglyceride and LDL cholesterol levels in humans. Further, the complete loss-of-function of Angptl3, which has been found naturally-occurring in certain families, does not appear to cause adverse health consequences in humans. Angptl3 is predominantly expressed in the liver, an ideal target organ for in vivo genome editing because of its tissue accessibility by intravenous injection. Together, these features make Angptl3 an attractive therapeutic target for genome editing. The genome editing enabled by delivery of Cas9 mRNA and gRNA with 306-O12B LNP occurs mainly in hepatocytes, as determined by the biodistribution of fluorescent signal in the Ai14 model mouse line. Moreover, the serum triglycerides and LDL-C levels are effectively reduced as a result of the hepatocyte-specific disruption of Angptl3.

The 306-O12B LNP led to more efficient genome editing than MC-3 LNP at the same dose of mRNA. Notably, a recent preclinical study of ASO-mediated silencing of Angptl3 in a similar mouse model achieved a 50% reduction in serum ANGPTL3 protein levels, 7% reduction of LDL-C, and 35% reduction in TG levels. A single dose of CRISPR-mediated knockdown using 306-O12B LNP matched or exceeded these knockdown benchmarks (65.2%, 56.8%, and 29.4% reductions, respectively), while MC3 LNP did not (25%, 15.7%, and 16.3% reductions, respectively). That particular ASO mouse study was followed up with a Phase I human clinical trial, suggesting the achieved levels of knockdown are likely therapeutically relevant. Direct comparisons of mouse study to human clinical trials for ANGPTL3 antibody or ASO therapies, however, are challenging due to the differences in species and dosing regimens. Clinical trials of both ASO and antibody therapies have demonstrated the ability to titer the percentage knockdown of these factors based on dosage amount and frequency, and in particular saw the strongest effects upon multiple repeated doses of drug per patient at the highest experimental dose level. A dose-dependent reduction in serum ANGPTL3 levels was observed upon a single injection of 306-O12B LNP. This indicates that in a clinical setting, LNP system could achieve similar dose- and frequency-dependent knockdown, and that the correct dosage scheme could achieve a similar magnitude of knockdown as reported in these clinical trials. The knockdown effects of a single dose, detectable at the genomic and protein levels, as well as in the downstream therapeutic metrics, were stable for at least 100 days. The human clinical trials indicated a maximum knockdown effect within 1 week of receiving the drug, with serum levels slowly returning to baseline over the subsequent months. While this result is expected, as CRISPR generates a permanent change in the target cell's genome while ASOs and antibodies are transient treatments, this difference is particularly notable, and may have tremendous therapeutic consequences.

In vivo CRISPR mRNA delivery may result in a genomic knockdown which is durable for at least 1 full year after administration. Although thorough analysis of serum lipid levels was only performed out to 100 days after administration, sequencing data indicated durable genomic editing at least to 150 days. Disclosed data indicates a very slight reduction in genomic editing efficiency over time, which may reflect the slow turnover of hepatocytes in the liver, which have a predicted life of approximately 300 days. The genomic editing efficiency of a 3 mg/kg dose detected at 150 days after injection (31%) is still higher than the editing efficiency of a lower 2 mg/kg dose detected at only 100 days after injection (22%). The 22% genome editing rate was sufficient to generate a statistically significant reduction of ANGPTL3 protein, as well as serum cholesterol and LDL-C. Thus, it is highly likely that the full suite of therapeutic metrics would also show a significant knockdown out to five months or beyond. This point warrants further study.

In addition to genome editing efficacy, off-target effects are a major concern for the clinical translation of CRISPR-Cas9 technology. Off-target effects can be discussed in two contexts: off-target delivery effects (i.e., the delivery of a liver drug to the kidneys) and biological off-target editing (i.e., editing events at unintended genomic loci). In this work, the luciferase and tdTomato model systems are used to demonstrate specific delivery to the liver and specifically hepatocytes, largely minimizing off-target delivery effects. To address the biological off-target editing issue, a highly specific sgRNA target to Angptl3 was designed by using publicly available computational-based design tools to mitigate off-target editing events. Targeted NGS data confirmed that no editing was detected in the liver at the top 9 off-target sites predicted by Cas-Off-Finder. While further studies, using techniques such as whole-genome sequencing and direct comparisons to viral-mediated CRISPR delivery or other guide sequences may be necessary to further validate the safety of disclosed platform for general liver delivery applications, the data presented here suggests that under the conditions examined, 306-O12B LNP were found to be highly specific with regards to both off-target delivery and off-target editing. From a systemic perspective, the LNP system did not cause any significant changes to ALT or AST levels, markers of liver health, and disclosed data indicates that the LNP are not detectably toxic to the liver. Similarly, the lack of significant change in TNF-alpha indicates that the LNPs delivery does not induce a significant immune response. Together, this indicates that disclosed treatment is safe in mice.

Outcomes of this study may advance the systemic delivery of CRISPR genome editing machinery in the clinic. To realize the final clinical application, more detailed preclinical studies for the chronic tolerability, off-target effects, and the efficacy in large animals should be performed in the future. Overall, 306-O12B LNP as a highly potent and safe non-viral delivery platform for clinically relevant liver-specific CRISPR-Cas9-based genome editing.

### Example 5: Synthesis of Lipidoids and In Vivo Screening of mRNA-Loaded LNPs

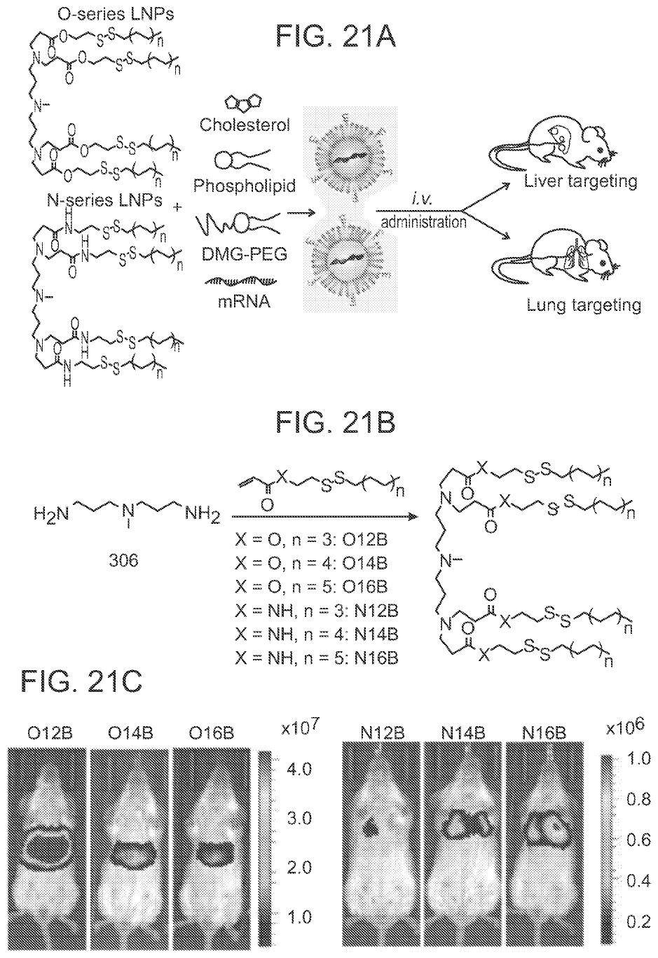

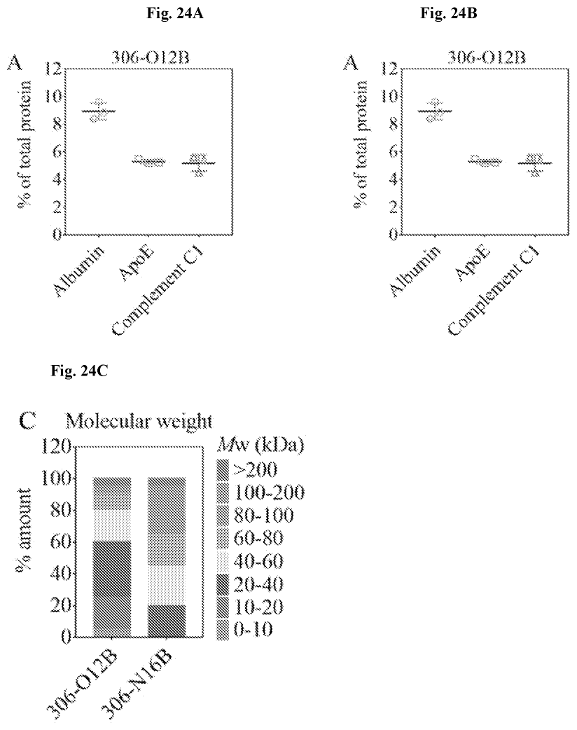

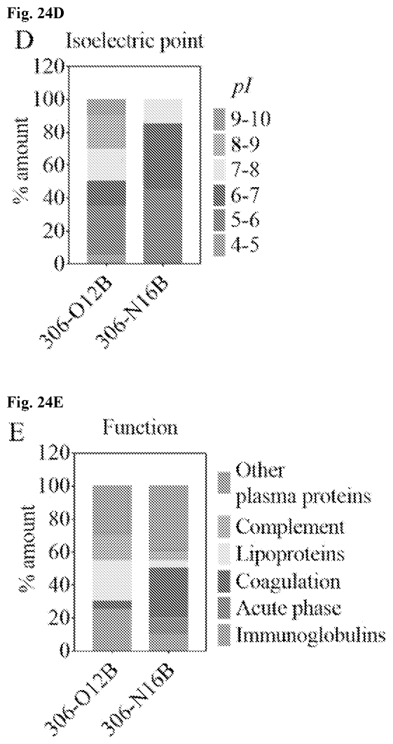

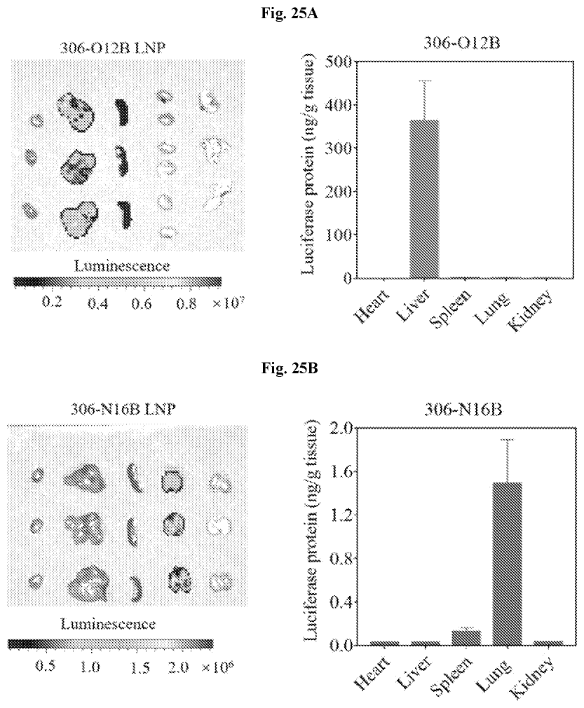

A set of bioreducible lipidoids were generated based on the particular amine head 3,3′-diamino-N-methyldipropylamine, referred to as 306 amine head, which has been previously demonstrated as a promising amine candidate for siRNA, ASO, and mRNA delivery in vivo. These lipidoids were created by reacting the 306 amine head with 3 ester bond incorporated alkyl-acrylate tails (0-series) and 3 alkyl-acrylate tails containing an amide bond (N-series) using a solvent-free Michael addition reaction (FIG. 21B). LNPs were then formulated with the previously optimized formulation condition of 306-O12B LNP for mRNA delivery with 50% synthetic active lipidoid, 38.5% cholesterol, 10% DOPC, and 1.5% DMG-PEG2000 in molar ratio. To investigate the in vivo systemic mRNA delivery profiles of these LNPs, firefly luciferase mRNA (fLuc mRNA) encoding was encapsulated for firefly luciferase, a reporter protein that can be easily visualized in vivo by using the IVIS imaging system (Perkin Elmer). The bioluminescence signals in the O-series LNPs (306-O12B, 306-O14B, and 306-O16B) treated mice were mainly located in the livers (FIG. 21A, FIG. 21C & FIG. 25A). Surprisingly, the fLuc mRNA was almost exclusively been delivered to the lungs by the N-series LNPs (306-N12B, 306-N14B, and 306-N16B) (FIG. 21A, FIG. 21C & FIG. 25B). Quantification of luciferase protein in different tissue homogenates further demonstrated the translation of luciferase-encoded mRNA with 306-O12B LNP and 306-N16B LNP was predominantly located in the liver and lung, respectively (FIG. 25A and FIG. 25B). In addition to the different organ-targeting profiles, the tail length showed exactly the opposite effect on the mRNA delivery efficacy of these two series LNPs. For the O-series LNPs, 306-O12B showed the highest potency, while in the N-series LNPs case, the efficacy trend was observed as 306-N16B>306-N14B>306-N12B.

### Example 6: A Second-Generation Library of N-Series LNPs Confirmed Tail Structure-Dependent Lung-Selectivity

Considering the fact that the amine head structure may also affect the physical-chemical property of lipidoids as well as the resulting LNPs, that will subsequently affect the in vivo fate of LNPs. The liver-targeted mRNA delivery allowed by the 0-series LNPs containing different amine and alkyl-tail structures has been extensively explored and demonstrated. Hence, the tail structure-dependent lung-targeted delivery of mRNA enabled by N-series LNPs using amines other than 306 was confirmed. Therefore, a secondary library of N-series lipidoids was synthesized by reacting the N16 tail with 80, 113, 304, 400, and 401 amines (FIG. 22A). The fLuc mRNA encapsulated LNPs with these lipidoids were prepared and investigated the in vivo organ-selectivity of the resulting LNPs. As shown in FIG. 22B, the luciferase bioluminescence signals were mainly observed in the lungs for all these N-series LNPs, confirming that the lung-selectivity of these LNPs is determined by the lipidoid linker tail structure, namely the amide linkage.

### Example 7: 306-N16B LNP Enables the Delivery of mRNA to the Pulmonary Endothelial Cells

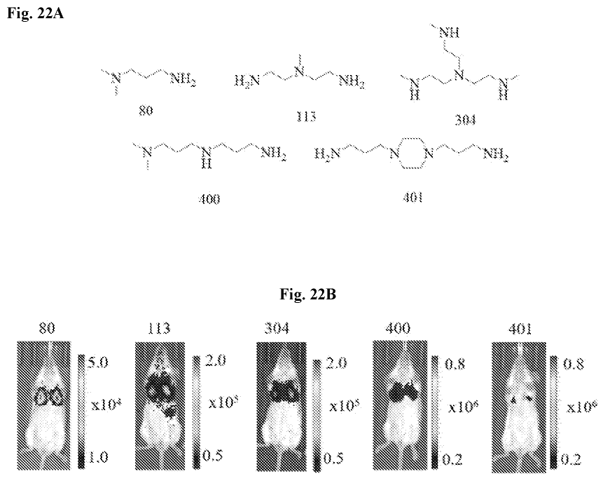

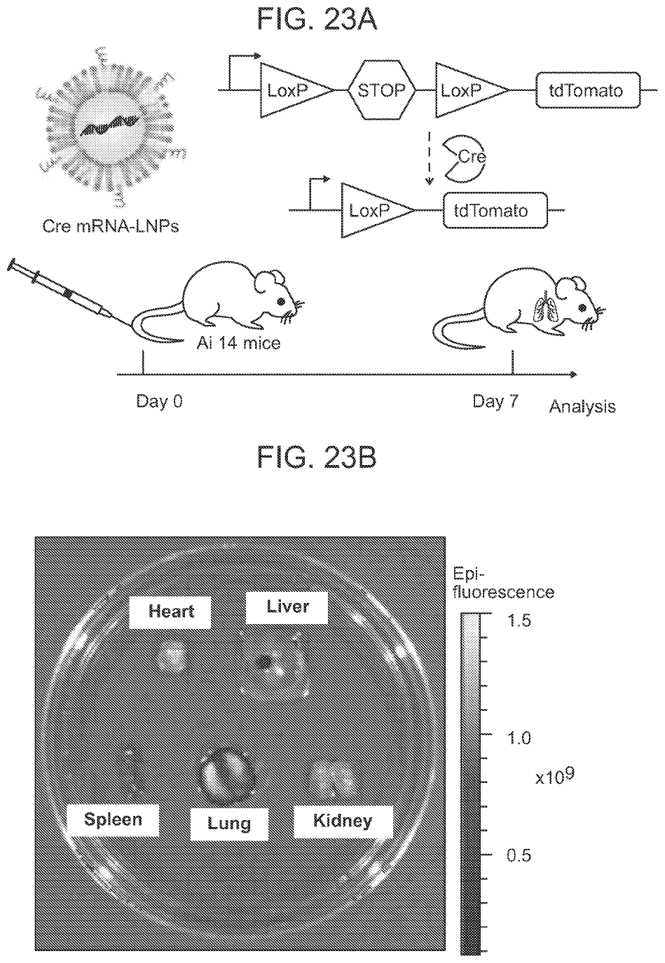

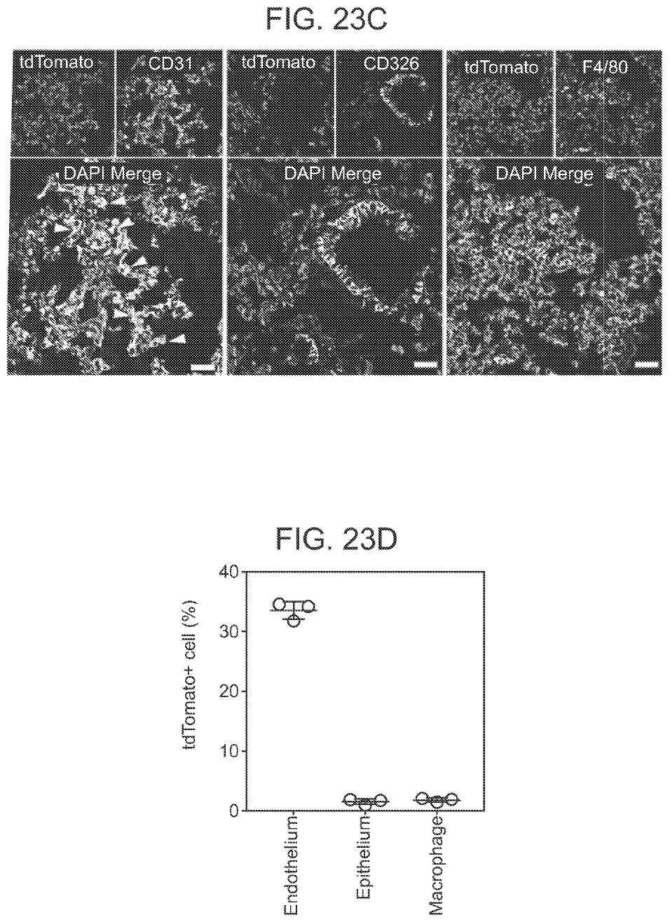

The clinical translation of mRNA-based therapeutics requires the specific mRNA delivery to the organs and cell types of interest. Given the ability of N-series LNPs to specifically deliver mRNA to the lungs, the primary organs of the respiratory system in humans. The lungs can be affected by many respiratory diseases caused by the dysfunction of endothelial, epithelial, and immune cells. Thus, it is critical to identify which lung cell subpopulation was specifically transfected for the further evaluation of the potential applications of disclosed lung-targeting LNPs in the treatment of pulmonary diseases. The genetically engineered Cre/LoxP Ai9 reporter mouse line that is widely used to achieve cell-type-specific expression of the tdTomato protein was utilized. The Ai9 mice was injected with 306-N16B LNP carrying the Cre mRNA, encoding Cre recombinase that cuts the stop cassette to activate the expression of tdTomato fluorescence signal in the edited cells (FIG. 23A). As shown in the ex vivo image of organs collected from the treated mice, the red tdTomato signal was detected specifically in the lung (FIG. 23B). The tissue sections were immune-stained with fluorescent dye-labeled antibodies against endothelial, epithelial, and macrophage cells to gain an initial insight on which specific subcellular population was edited in the lung. Interestingly, the red tdTomato signal was mainly localized in the pulmonary endothelial cells, while little colocalization with epithelial or macrophage cells was observed (FIG. 23C). To further quantify the transfected specific cell types in the lung, lung tissues were proceeded into single-cell suspension and analyzed using flow cytometry. ˜33.6% of pulmonary endothelium was transfected, while a very small portion of the epithelium (˜1.66%) and macrophage cells (˜1.92%) were transfected (FIG. 23D). These findings suggest that the 306-N16 LNP is capable of selectively deliver mRNA to the pulmonary endothelial cells, which is important for the further development of potential therapeutic applications against lung diseases associated with the endothelial cells.

### Example 8: 306-N16B LNP Allows Lung-Specific Delivery of Genome Editing Cas9 mRNA

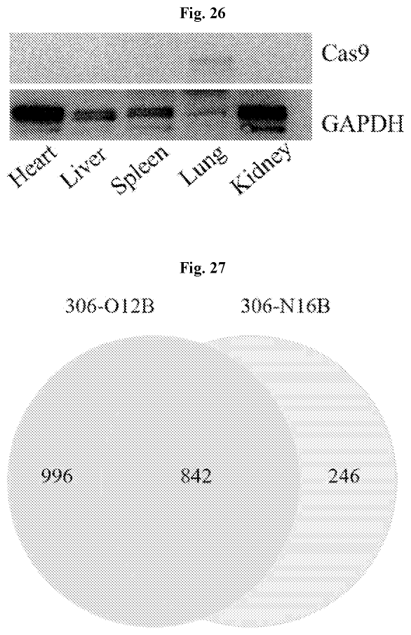

As demonstrated above that the 306-N16B LNP is capable of specifically deliver fLuc mRNA and Cre mRNA to the lungs, and the length of these two mRNAs is less than 2000 nucleotides (1960 nucleotides for fLuc mRNA and 1350 nucleotides for Cre mRNA). Some specific diseases may require the delivery of even larger mRNA molecules. It was tested whether 306-N16B LNP could similarly have the potential to deliver longer mRNA to the lung. 306-N16B LNP was formulated with Cas9 mRNA, a genome editing mRNA that has 4521 nucleotides which is more than three times longer than the fLuc mRNA, the LNP was then injected into the Babl/c mice. After 6 h, mice were sacrificed, and organs were collected for further western blot analysis to detect the Cas9 protein expression in different organs. Notably, the Cas9 protein can be only detected in the lung (FIG. 26), indicating that 306-N16B LNP-mediated specific delivery of Cas9 mRNA to the lung. The 306-N16B LNP holds tremendous potential in the application of treating genetic pulmonary diseases via specific genome editing.

### Example 9: Characterization of Proteins Constituting the Protein Corona in 306-O12B LNP and 306-N16B LNP

It is believed that the outer surface of nanoparticles (NPs) is immediately masked with a layer of biomolecular corona shortly after intravenous administration, which can dramatically alter the surface properties of NPs and determine theirs in vivo fate. The controlled adsorption of specific plasma proteins on the NPs surface would serve as targeting molecules that could selectively direct NPs to specific organs. For the dramatically different in vivo organ-targetability of 306-O12B and 306-N16B LNPs, It might be attributed to the serum proteins formed on their surfaces. To identify and quantify the proteins on the 306-O12B and 306-N16B LNPs, these two LNPs were incubated with mouse plasma at 37° C. for 1 h, and isolated and recovered the protein-coated LNPs using centrifugation followed by extensive washing with PBS. Proteomics was then performed to analyze the composition of proteins on LNPs. 1838 and 1088 proteins were identified and quantified on the 306-O12B LNP and 306-N16B LNP, respectively. Venn diagrams showed the common and unique proteins between 306-O12B LNP and 306-N16B LNP (FIG. 27). The top-20 most-abundant corona proteins, which may play the dominant role in the protein corona, for each LNP are listed in Table 1 above. 306-O12B LNP and 306-N16B LNP only shared 6 common proteins among the top-20 proteins, which demonstrated the proteins absorbed on the LNP surface play a key role in determining the in vivo targetability of LNPs. Previous studies reported apolipoprotein E (ApoE)-mediated delivery of LNPs to the liver. ApoE was identified as the second dominant protein composition in the corona of liver-targeting 306-O12B LNP (FIG. 24A), which was in line with the literature reports. On the contrary, the top-3 protein compositions in the corona of lung-targeting 306-N16B LNP are serum albumin, fibrinogen beta chain, and fibrinogen gamma chain (FIG. 24B). It has been reported that the coating of fibrinogen could improve the endothelial cell adhesion as well as the endothelialization. Whether 306-N16B LNP-mediated delivery of mRNA to the lung endothelium was via a fibrinogen-dependent mechanism, however, remains to be further proved.

The top-20 proteins are classified according to their molecular weight (Mw) (FIG. 24C). A slight difference between these two LNPs was observed, the protein corona of 306-O12B LNP was enriched in low molecular weight proteins that 80% of proteins have had an Mw<60 kDa. However, 55% of proteins in the protein corona of 306-N16B LNP are larger than 60 kDa. The proteins were categorized based on their isoelectric point (pI). As shown in FIG. 24D, 50% and 80% of proteins displaying a negative charge (pI<7) at physiological pH 7.4 constitute the protein corona of 306-O12B LNP and 306-N16B LNP, respectively. It was suggested that positively charged NPs prefer to attract negatively charged proteins (M. P. Monopoli et al., Physical-Chemical Aspects of Protein Corona: Relevance to in Vitro and in Vivo Biological Impacts of Nanoparticles. J. Am. Chem. Soc. 133, 2525-2534 (2011)). However, significant difference of the zeta potential of two LNPs was not observed, both LNPs showed slight negative surface charge (Table 2), indicating that surface charge may not be the only factor that affects the interaction between nanoparticles and proteins in the biological fluids. The proteins concerning their biological functions were further sorted as shown in FIG. 24E. From the function point of view, it was found that proteins abundant on 306-O12B LNP and 306-N16B LNP serve very similar functions, they are all associated with the function of lipid metabolism, complement activation, immune responses, acute-phase response, and coagulation. It should be noted, however, that aside from the other plasma proteins, the highest enriched proteins in the corona of 306-O12B LNP are the apolipoproteins that involve in lipid and cholesterol metabolism (FIG. 24E). While coagulation-relevant corona proteins represent the largest fraction in the corona of 306-N16B LNP (FIG. 24E).

TABLE 2
Characterization of fLuc mRNA loaded LNP formulations^{a}.

| LNPs | Size (nm) | PDI | Zeta (mV) |
|---|---|---|---|
| 306-O12B | 117 | 0.19 | −8.7 |
| 306-N16B | 81 | 0.20 | −5.0 |

^{a}The size, PDI, and zeta potential of LNPs were measured using the ZetaPALS DLS machine (Brookhaven Instruments).

Collectively, these findings illustrated that the protein coronas formed on these two 306-O12B LNP and 306-N16B LNP differ in protein compositions, fractions, and biological functions, which play the dominant role in determining the in vivo fate of these two LNPs.

### Discussion

In recent years, increasing interest was seen in using the LNPs to deliver messenger RNA (mRNA) therapeutics. The in vitro synthetic mRNA has the potential to produce therapeutically relevant proteins “in vivo” to control and treat a broad spectrum of diseases, including AIDS, Zika, rare diseases, cancer, and coronavirus. To leveraging this transformative technology to benefit human health, the mRNA needs to be sent to the diseased organs and cells to enable specific expression of therapeutic proteins, and subsequently, create a desired therapeutic effect. Lipid nanoparticles (LNPs) represent one of the leading non-viral delivery systems for nucleic acid delivery and considerable efforts have been made to employ LNPs for mRNA delivery. However, the fact is that the majority of developed LNPs have targeted the liver after systemic administration, therefore new strategies are demanded to direct LNPs to other organs, such as the lung, kidney, heart, and brain. It is known that some LNPs containing specific amine heads, such as imidazole, adamantly, and neuron transmitter, delivered mRNA, siRNA, and ASO to spleen and brain. These LNPs were identified through a labor-intensive screening approach, and it is currently still impossible to generate a common rule to guide the rational design of LNPs with predictable organ-selectivity. More recently, the Siegwart lab discovered and developed a selective organ targeting (SORT) strategy to engineer LNPs to target extrahepatic tissues, for example the lung and spleen, with the adding of an extra excipient. This advance is exciting, but it made the original four components system to five components that somehow unfavorably complicated the formulation.

Disclosed herein is the in vivo organ-selectivity of LNPs can be precisely tuned by simply changing the linker structure in the lipidoid tails without complicating the LNPs formulation. By changing the linker from ester bond (referred as O-series) to amide bond (referred as N-series), the mRNA delivery specificity enabled by LNPs was switched from the liver to the lung. This finding was first observed from the 306-amine head-based O-series and N-series LNPs. By examining the mRNA delivery targetability of a secondary library of N-series LNPs, it was confirmed that the lung-targeting ability enabled by amide bond linkage is generally applicable to other classes of amine heads. Targeted RNA delivery to the lung is an attractive concept, which offers tremendous opportunities to target currently be defined as “undruggable” targets for the treatment a variety of pulmonary diseases. In this study, it is demonstrated that 306-N16B LNP can specifically transport mRNA to the pulmonary endothelial cells, an important therapeutic target that involves in acute respiratory distress syndrome, inflammation, and thrombosis.

Proteomic study was performed to try to explain why such a small structural difference between the O-series and N-series LNPs leads to dramatically different organ selectivity. These two tail structures may have specific affinity to some distinct serum proteins that would somehow regulate the protein compositions of coronas formed on the surface of the LNPs during the circulation. It has been reported that the protein corona plays a key role in determining the in vivo fate of nanoparticle. Disclosed results revealed that the number and type of absorbed plasma proteins in the protein coronas of 306-O12B and 306-N16 LNPs are different. Only 6 common proteins were found among the top 20 most abundant proteins in these two LNPs. Even there are same sets of proteins are found in both protein coronas, they may have totally different biological functions because they are randomly oriented in the protein corona layer. One example is the ApoE, on one hand, it has been widely reported that endogenous ApoE could be absorbed to the LNPs surface following i.v. injection to facilitate the transport of encapsulated RNAs to hepatocytes by lipoprotein receptor-mediated endocytosis, while on the other hand, ApoE could also be deployed to direct nanoparticles to the brain.

In summary, this study demonstrated a strong correlation of the structure-activity relationships between lipidoid tail chemistry and organ selectivity for mRNA delivery. The mechanistic study using proteomics indicated that the protein composition in the protein coronas of these two LNPs are different, suggesting the unique tail structures may influence the interaction of LNPs and biological fluids that may have an impact on the targetability of LNPs. However, further studies should be performed to fully elucidate the effect of these unique proteins on organ-targetability. The findings from this work provide a fundamental understanding of how lipid structure may affect the in vivo fate of LNPs by manipulating protein corona functions.

### Example 10: Synthesis of N-Series Lipidoids and In Vivo Screening of mRNA-Loaded LNPs

Nine bioreducible N-series lipidoids were generated by reacting the acrylamide tails (N16B, N14B, and N12B) with different commercially available amine heads using a solvent-free Michael addition reaction (FIG. 28A). LNPs were then formulated with the previously optimized formulation condition of LNP for mRNA delivery with 50% synthetic active lipidoid, 38.5% cholesterol, 10% DOPC, and 1.5% DMG-PEG2000 in molar ratios. To investigate the in vivo systemic mRNA delivery profiles of these LNPs, firefly luciferase mRNA (fLuc mRNA) encoding firefly luciferase was encapsulated, a reporter protein that can be visualized in vivo using the IVIS imaging system (Perkin Elmer). Interestingly, the bioluminescence signals in the N-series LNPs treated mice were mainly located in the lungs (FIG. 28B). In addition, it was found that the chemical structure of lipidoids plays a critical role in determining the in vivo mRNA delivery efficacy of the formulated LNPs. As shown in FIG. 28C, the quantified luciferase bioluminescence intensity in the lungs at 6 h post-injection demonstrates that LNPs incorporated with the 306 amine head exhibited the highest delivery efficacy, as compared with the 304 and 113 LNPs. The delivery efficacy also increases with the increasing of the tail length of lipidoids, lipidoids armed with the N16B tail exhibited the highest potency than that with N14B and N12B tail.

### Example 11: Targeting Different Pulmonary Subcellular Populations by Changing the Amine Head Structure of N-Series LNPs

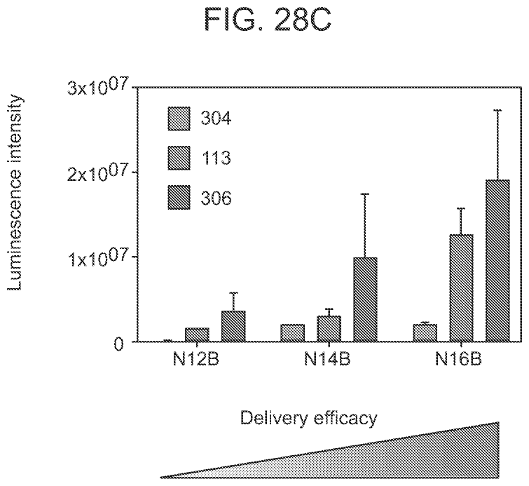

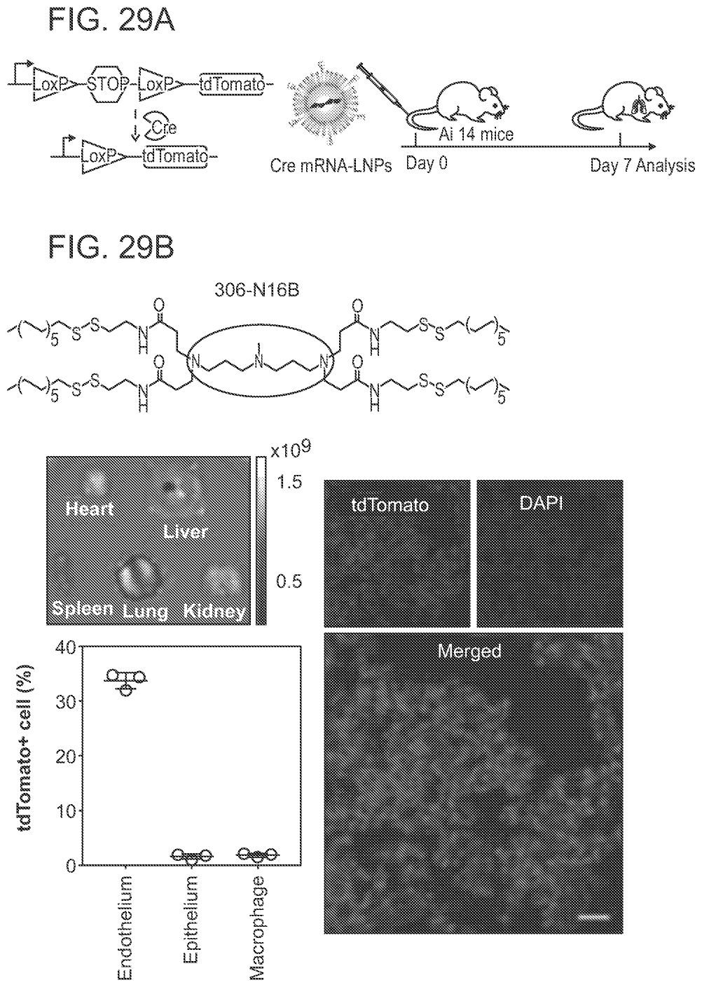

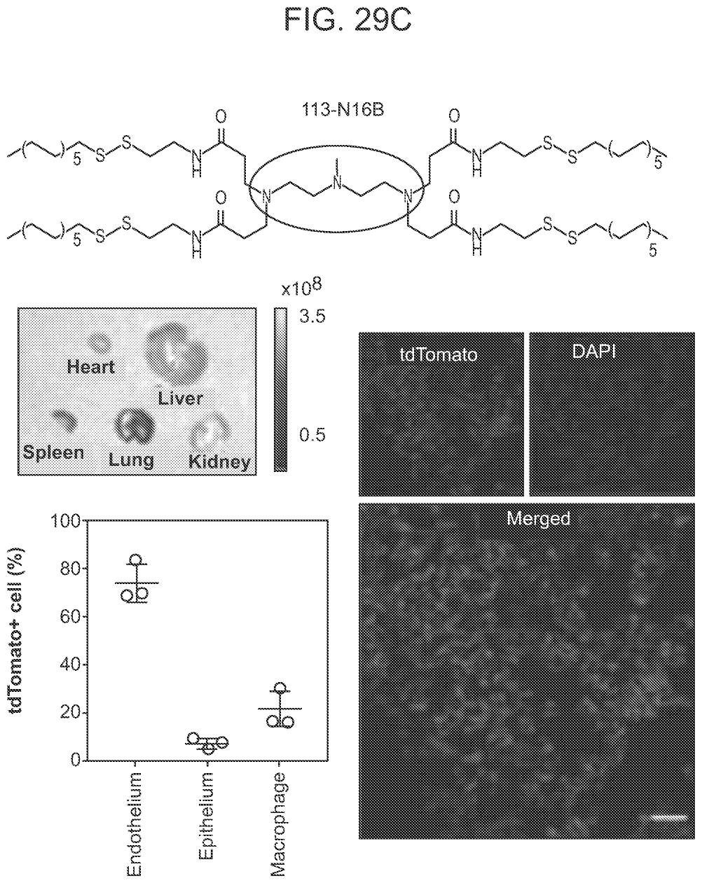

The clinical translation of mRNA-based therapeutics requires the specific mRNA delivery to the organs and cell types of interest. The lungs can be affected by many diseases caused by dysfunction of multiple cellular compartments, including endothelial, epithelial, and immune cells. To identify the lung cell subpopulations that are specifically transfected, the genetically engineered Cre/LoxP Ai14 reporter mouse line was used to achieve cell-type-specific expression of the tdTomato protein. The Ai14 mice was injected with the two most potent lung-targeting LNPs (306-N16B and 113-N16B). Cre mRNA, encoding Cre recombinase, was used as an mRNA cargo in these experiments. Once delivered into the cells, Cre recombinase removes the stop cassette and activates the expression of tdTomato fluorescence signal in the edited cells (FIG. 29A). As shown in the ex vivo image of organs collected from the treated mice (FIGS. 29B & 29C), the red tdTomato signal was detected specifically in the lungs of both 306 and 113-N16B LNP treated mice, and tdTomato positive cells were observed by using confocal imaging of lung sections. To further identify and quantify the transfected specific cell types in the lungs, lung tissues were processed into single-cell suspensions and analyzed using flow cytometry. For the 306-N16B LNP, 33.6% of pulmonary endothelium was transfected, compared with 1.5% of epithelium and 1.9% of macrophages (FIG. 29B), indicating that the 306-N16 LNP is capable of selectively deliver mRNA to the pulmonary endothelial cells. In contrast, 113-N16B LNP delivered Cre mRNA preferentially to endothelial cells (69.6% of total endothelial cells were transfected), but also to macrophages (18.9%) and epithelium (7.3%) (FIG. 29C). These findings demonstrate that different pulmonary cell populations can be targeted by simply tuning the head structure of N-series LNPs, setting the stage for potential therapeutic applications for lung diseases associated with specific pulmonary cells.

### Example 12: Characterization of Proteins Constituting the Protein Corona in 306-O12B LNP and 306-N16B LNP

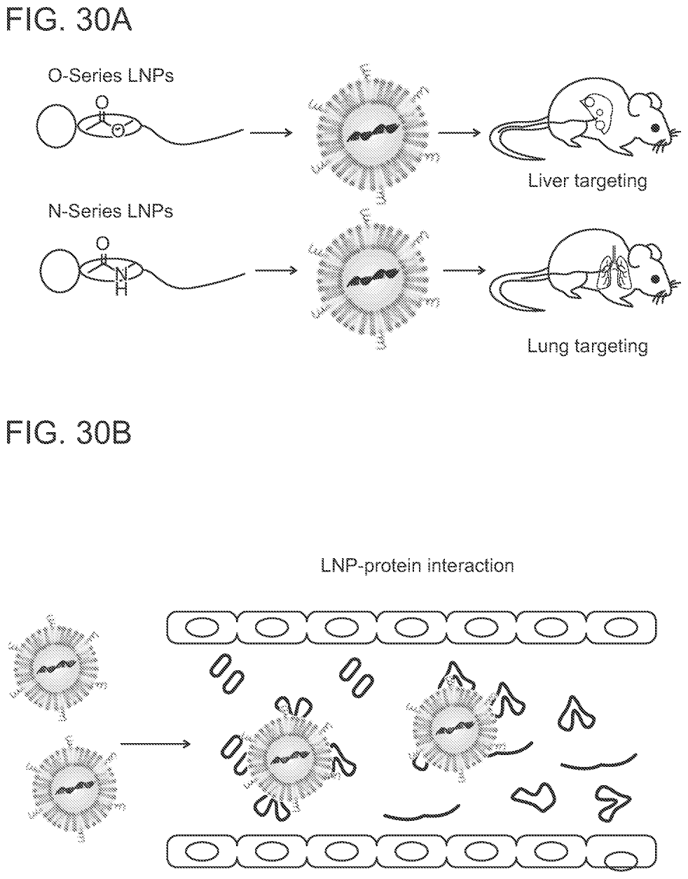

It is believed that the outer surface of nanoparticles (NPs) is immediately masked with a layer of biomolecular corona shortly after intravenous administration, which can dramatically alter the surface properties of NPs and determine their in vivo fate. The controlled adsorption of specific plasma proteins on the NPs surface may selectively direct NPs to specific organs. It was previously reported that O-series LNPs could specifically deliver mRNA to the liver (M. Qiu et al., Lipid nanoparticle-mediated codelivery of Cas9 mRNA and single-guide RNA achieves liver-specific in vivo genome editing of Angptl3. Proc. Natl. Acad. Sci. U.S.A. 118, e2020401118 (2021)). The dramatically different in vivo organ-targetability of O- and N-series LNPs (FIG. 30A) might be attributed to the serum proteins formed on their surfaces (FIG. 30B). To test this, the proteins on two representative LNPs, 306-O12B and 306-N16B were identified and quantified. These two LNPs were incubated with mouse plasma at 37° C. for 1 h, and isolated and recovered the protein-coated LNPs using centrifugation followed by extensive washing with PBS. Proteomics was then performed to analyze the composition of proteins on LNPs. 1838 and 1088 proteins were identified and quantified on the 306-O12B LNP and 306-N16B LNP, respectively.

The top 20 most-abundant corona proteins are listed, which may play the dominant role in the protein corona, for each LNP in Table 3. 306-O12B LNP and 306-N16B LNP only shared 6 common proteins among the top 20, supporting that the proteins absorbed on the LNP surface may participate in the in vivo targetability. ApoE was identified as the second dominant protein composition in the corona of liver-targeting 306-O12B LNP (FIG. 24A), consistent with a prior study demonstrating apolipoprotein E (ApoE)-mediated delivery of LNPs to the liver. In contrast, the top-3 proteins in the corona of lung-targeting 306-N161B LNP are serum albumin, fibrinogen beta chain, and fibrinogen gamma chain (FIG. 24B). It has been reported that fibrinogen coating can improve endothelial cell adhesion and endothelialization.

TABLE 3
Top 20 most-abundant corona proteins identified
in the protein corona of LNPs

| No. | 306-O12B | 306-N16B |
|---|---|---|
| 1 | Albumin | Albumin |
| 2 | Apolipoprotein E | Fibrinogen beta chain |
| 3 | Complement C1q subcomponent | Fibrinogen gamma chain |
| | subunit B | |
| 4 | Immunoglobulin heavy constant | Fibrinogen alpha chain |
| | mu | |
| 5 | Apolipoprotein A-I | Fibronectin |
| 6 | Complement C1q subcomponent | Ceruloplasmin |
| | subunit C | |
| 7 | Complement C1q subcomponent | Immunoglobulin heavy constant |
| | subunit A | alpha (Fragment) |
| 8 | Apolipoprotein B-100 | Prothrombin |
| 9 | Beta-globin | Immunoglobulin heavy constant |
| | | mu |
| 10 | Apolipoprotein A-IV | Complement C3 |
| 11 | Apolipoprotein C-I | H-2 class I histocompatibility |
| | | antigen, Q10 alpha chain |
| 12 | Serotransferrin | Inter-alpha-trypsin inhibitor |
| | | heavy chain H3 |
| 13 | Pregnancy zone protein | Carboxylesterase 1C |
| 14 | Immunoglobulin heavy constant | Apolipoprotein A-I |
| | alpha (Fragment) | |
| 15 | Immunoglobulin kappa constant | Vitronectin |
| 16 | Immunoglobulin kappa variable | Inter-alpha-trypsin inhibitor |
| | 17-121 (Fragment) | heavy chain H2 |
| 17 | Fibrinogen beta chain | Protein AMBP |
| 18 | Alpha globin 1 | Inter-alpha-trypsin inhibitor |
| | | heavy chain H1 |
| 19 | Immunoglobulin heavy constant | Alpha-1-antitrypsin 1-4 |
| | gamma 2C (Fragment) | |
| 20 | Vitronectin | Bradykinin |

Proteins highlighted in bold are unique proteins among the top 20 for indicated LNPs.

The top 20 proteins were further classified according to their molecular weight (Mw) (FIG. 24C). The protein corona of 306-O12B LNP was enriched in low molecular weight proteins with 80% of proteins having an Mw<60 kDa, while 55% of proteins in the protein corona of 306-N161B LNP are larger than 60 kDa. The proteins were also categorized based on their isoelectric point (pI). As shown in FIG. 24D, 50% and 80% of proteins displaying a negative charge (pI<7) at physiological pH 7.4 constitute the protein corona of 306-O12B LNP and 306-N16B LNP, respectively.

Finally, the proteins were sorted according to their biological functions (FIG. 24E). Proteins abundant on 306-O12B LNP and 306-N16B LNP are associated with lipid metabolism, complement activation, immune responses, acute-phase response, and coagulation. It should be noted, however, that aside from the other plasma proteins, the highest enriched proteins in the corona of 306-O12B LNP are the apolipoproteins that involve in lipid and cholesterol metabolism (FIG. 24E), while coagulation-relevant corona proteins represent the largest fraction in the corona of 306-N16B LNP (FIG. 24E). Collectively, these findings illustrated that the protein coronas formed on 306-O12B LNP and 306-N16B LNP differ in protein compositions, fractions, and biological functions, which may play critical roles in determining their in vivo tissue targeting.

### Example 13: 306-N16B LNP Allows Lung-Specific Delivery of Genome Editing Cas9 mRNA

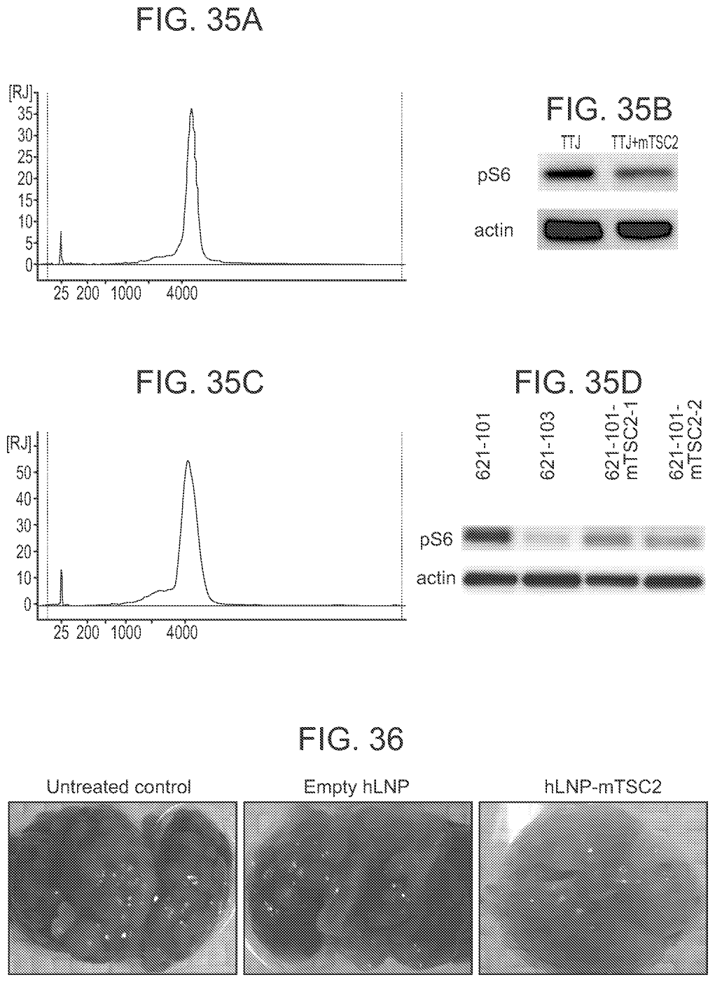

As demonstrated herein, the 306-N16B LNP is capable of specifically delivering fLuc mRNA and Cre mRNA to the lungs. The length of these two mRNAs is less than 2000 nucleotides (1960 nucleotides for fLuc mRNA and 1350 nucleotides for Cre mRNA). Some specific diseases may require the delivery of even larger mRNA molecules. It was tested whether 306-N16B LNP could similarly have the potential to deliver longer mRNA to the lung. 306-N16B LNP was formulated with Cas9 mRNA, a genome editing mRNA that has 4521 nucleotides which is more than three times longer than the fLuc mRNA, the LNP was then injected into the Babl/c mice. After 6 h, mice were sacrificed, and organs were collected for further western blot analysis to detect the Cas9 protein expression in different organs. Notably, the Cas9 protein can be only detected in the lung (FIG. 35), indicating that 306-N16B LNP-mediated specific delivery of Cas9 mRNA to the lung.

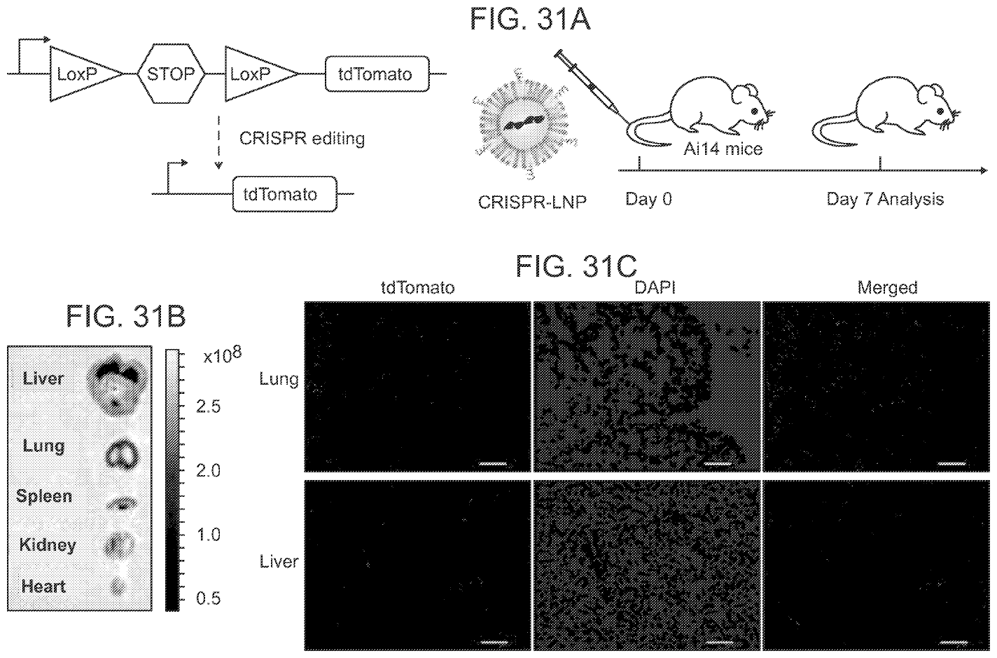

After confirmed that 306-N16B LNP could specifically deliver Cas9 mRNA to the lungs, it was tested if Cas9 mRNA and s single guide RNA in 306-N16B LNP can be co delivered to the lungs to enable lung-specific CRISPR-Cas9 genome editing. To test this, injected Ai14 mice with Cas9 mRNA and a LoxP-targeting sgRNA (sgLoxP) loaded 306-N16B LNP at a dose of 1.67 mg/kg of total RNA (Cas9 mRNA/sgLoxP=1/1.2, wt/wt) and analyzed gene editing specificity at day 7 post-injection (FIG. 31A). As shown in FIG. 31B, fluorescence signals were mainly seen in the lungs, a weak fluorescence signal was also observed in the liver. tdTomato positive cells were seen from the microscopy images of tissue sections (FIG. 31C). The results indicated that 306-N16B LNP was able to co-deliver Cas9 mRNA and sgRNA to both lungs and liver in mice to allow genome editing applications. However, it was found that 306-N16B LNP showed less tissue selectivity in co-delivering Cas9 mRNA and sgRNA as compared with the single mRNA delivery that could almost exclusively deliver mRNA to the lungs. This indicates that the incorporation of a shorter sgRNA could alter the physical property of formed LNP and change the organ-tropism of LNP. Including additional components into the LNP formulation can change the delivery efficiency and tropism.

### Example 14: LNP for Lung-Targeted TSC2 mRNA Delivery for the Treatment of LAM In Vivo

Pulmonary lymphangioleiomyomatosis (LAM) is a rare genetic lung disease that is characterized by aberrant mTORC1 hyperactivity caused by inactivating mutations of the Tuberous Sclerosis Complex 1 or 2 (TSC1 or TSC2) genes. Rapamycin (sirolimus), an mTORC1 inhibitor, is FDA-approved for the treatment of LAM, although ongoing loss of lung function and need for lung transplantation can still occur. Thus, there is an urgent need to develop new therapies for the treatment of LAM. Therefore, it was evaluated whether the lung targeting LNPs can deliver mRNA encoding the TSC2 gene for the treatment of LAM.

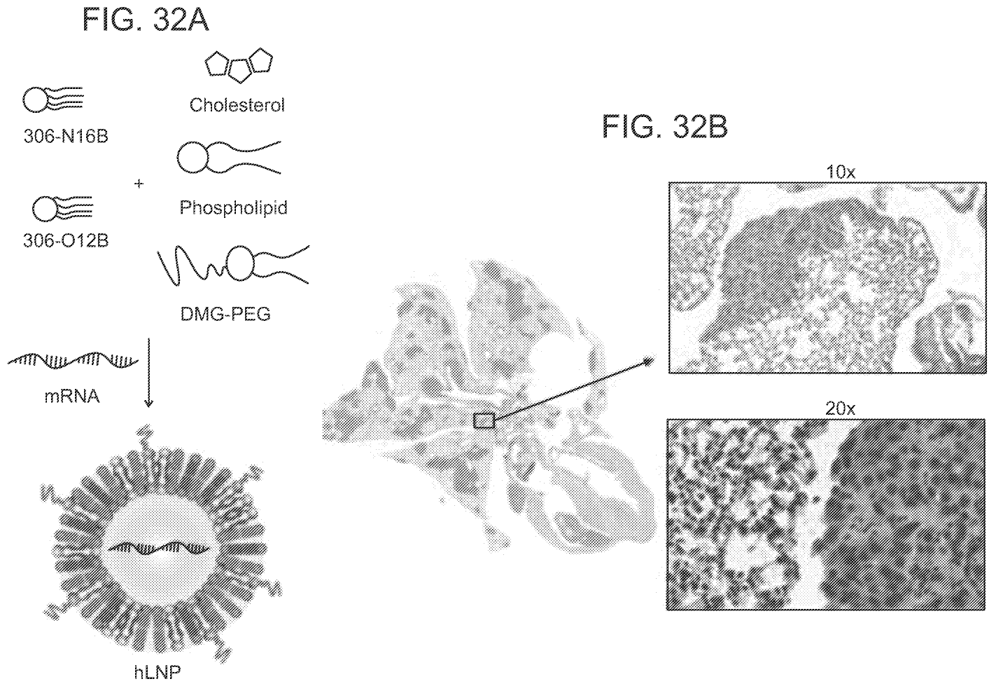

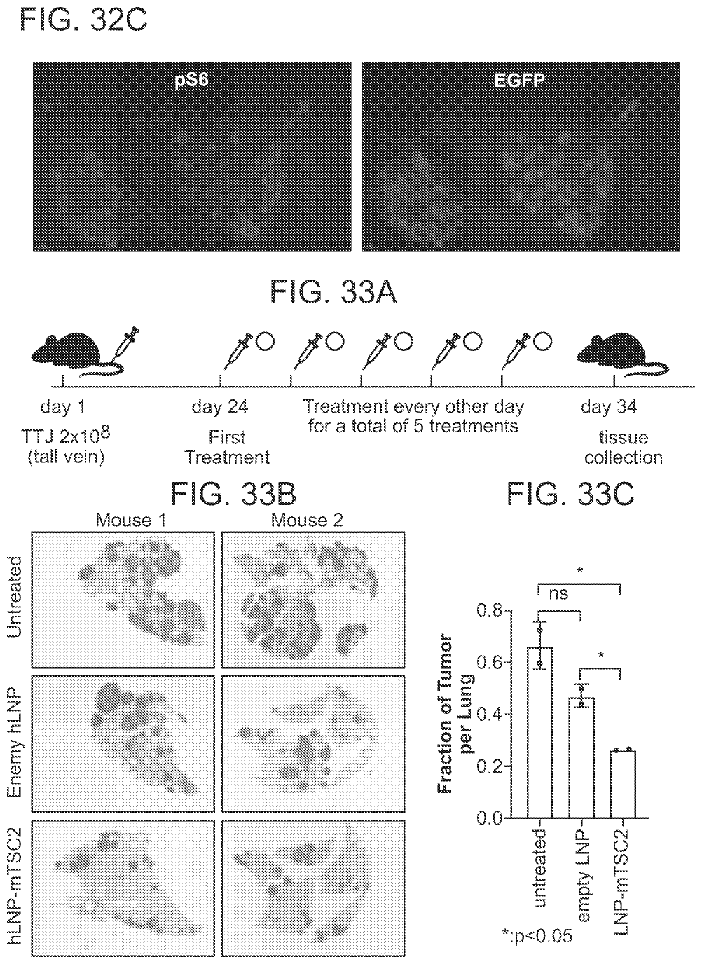

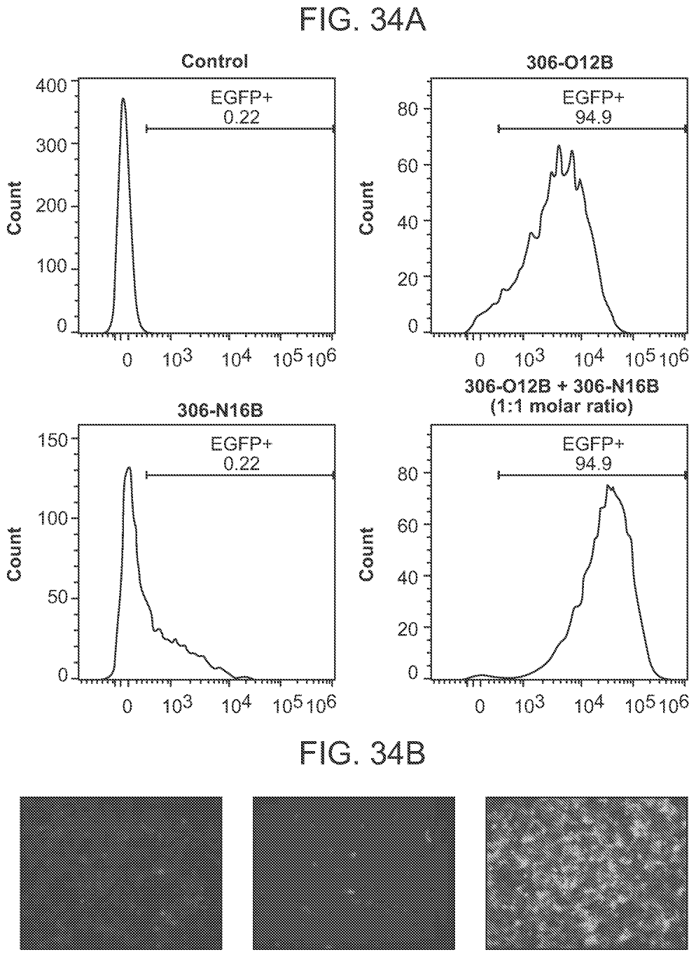

The feasibility of the LNPs was tested to deliver EGFP mRNA into mouse TSC2-null TTJ cells, which are used in an established model of LAM. In vitro, it was found that the lung targeting 306-N16B LNP showed low delivery efficacy in transporting mRNA to TTJ cells, but the liver targeting 306-O12B LNP exhibited a relatively higher transfection efficiency (FIG. 34A). Therefore, a hybrid LNP formulated with 50% of 306-O12B and 50% 306-N16B in molar ratio (referred to as hLNP) was designed (FIG. 32A). Such hybrid LNP can efficiently deliver cargos to the TTJ cells and also achieve lung targeted delivery in vivo. Interestingly, such hLNP showed a much higher transfection efficiency towards TTJ cells when compared with 306-O12B and 306-N16B LNPs, and uniform expression of EGFP was observed (FIG. 34B).

To determine whether hLNP can deliver mRNA to TSC2-deficient cells in vivo, TTJ cells were injected intravenously into syngeneic 6-week-old C57BL/6J mice to form LAM-like nodules in the lung as previously reported. hLNP encapsulating EGFP mRNA were injected via tail vein. The mice was sacrificed and collected the lungs after 6 hours after injection. EGFP expression in the lung was analyzed through immunohistochemistry (IHC). Remarkably, as shown in FIG. 32B, EGFP expression was primarily detected in TTJ-derived tumor nodules but not in adjacent normal lung tissue. Phospho-S6 (a marker of TSC2-deficient cells) immunostaining revealed strong co-localization of phospho-S6 and EGFP (FIG. 32C), confirming that hLNP enables specific delivery of EGFP mRNA to TTJ cells.

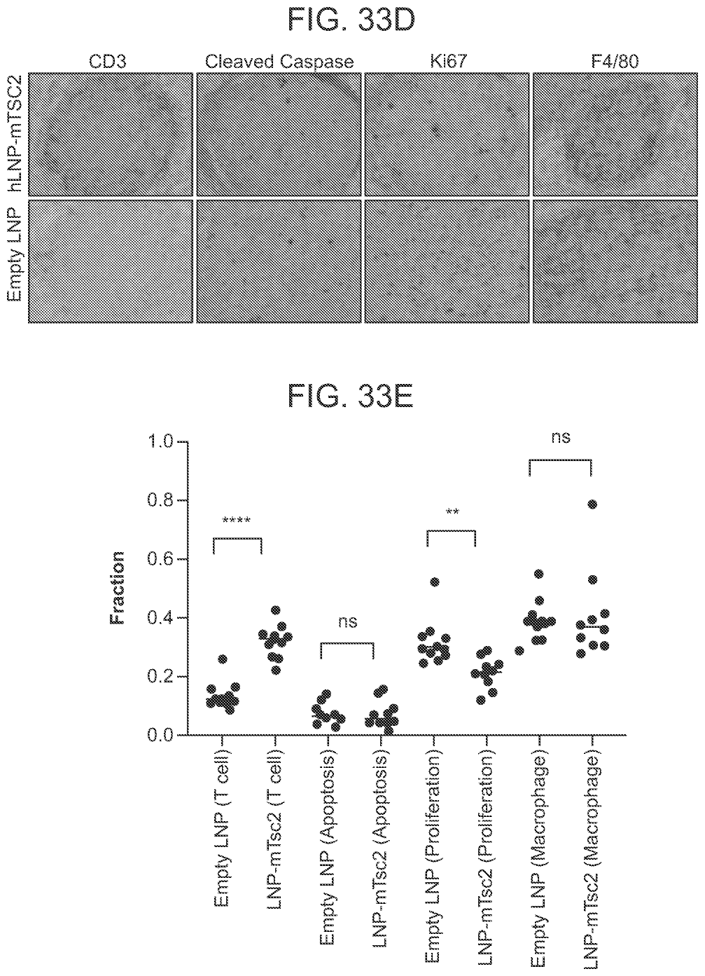

Next was evaluated the therapeutic effect of hLNP loaded with Tsc2 mRNA in this preclinical model. Full-length (5445 nucleotides) mouse wild-type Tsc2 mRNA was transcribed in vitro. In vitro, this mRNA suppressed the expression of phospho-S6 in TSC2-null cells (FIGS. 26A-26B), indicating successful suppression of the mTORC1 pathway. Similarly, re-expressing wild-type TSC2 mRNA in patient-derived TSC2-deficient cells significantly inhibited mTORC1 signaling (FIGS. 26C-26D). TTJ tumor-bearing mice was treated with Tsc2 mRNA encapsulated hLNP (0.75 mg/kg) or empty hLNP, every other day for a total of 5 doses, starting on day 24 after TTJ cell inoculation, with 2 mice in each group. Mice without treatment were also included as a control (FIG. 33A). The Tsc2 mRNA-loaded hLNP significantly suppressed tumor growth compared to that of empty LNPs or without treatment (FIG. 36). The overall size of tumor nodules in mice treated with Tsc2 mRNA LNP was reduced by 60% or 44% compared to that of the untreated group or the group treated with empty LNP, respectively (FIGS. 33B & 33C). No significant difference was observed between the empty LNP treatment group and the untreated group. Further evaluated was the tumor proliferation and apoptosis using IHC against Ki67 and cleaved caspase 3, respectively (FIGS. 33D & 33E). Cell proliferation within tumor modules was decreased by Tsc2 mRNA LNP treatment compared to that of empty LNP. No significant difference in apoptosis was observed between the two groups, suggesting that tumor apoptosis is not a major mechanism of tumor reduction in this model. Wildtype TSC2 restoration through mRNA delivery may thus induce immune-mediated mechanisms that reduce the TTJ cells tumor burden. To test this possibility, macrophage and T cell tumor infiltration by IHC against F4/80 and CD3 was assessed. No difference in macrophage infiltration was observed between the groups, but the Tsc2 mRNA-loaded hLNP treatment group showed enhanced tumor T cell infiltration (FIG. 33E), demonstrating that restoration of TSC2 protein expression induces an immune mediated therapeutic response in this preclinical model of LAM.

### Discussion

In recent years, increasing interest was seen in using the LNPs to deliver messenger RNA (mRNA) therapeutics. The in vitro synthetic mRNA has the potential to produce therapeutically relevant proteins “in vivo” to control and treat a broad spectrum of diseases, including AIDS, Zika, rare diseases, cancer, and coronavirus. To leveraging this transformative technology to benefit human health, the mRNA needs to be sent to the diseased organs and cells to enable specific expression of therapeutic proteins, and subsequently, create a desired therapeutic effect. Lipid nanoparticles (LNPs) represent one of the leading non-viral delivery systems for nucleic acid delivery and considerable efforts have been made to employ LNPs for mRNA delivery. However, the fact is that the majority of developed LNPs have targeted the liver after systemic administration, therefore new strategies are demanded to direct LNPs to other organs, such as the lungs, kidneys, heart, and brain. These LNPs were identified through a labor-intensive screening approach, and it is currently still impossible to generate a common rule to guide the rational design of LNPs with predictable organ-selectivity. More recently, the Siegwart lab discovered and developed a selective organ targeting (SORT) strategy to engineer LNPs to target extrahepatic tissues, for example, the lung and spleen, with the adding of an extra excipient. This advance is exciting, but it made the original four components system into five components that somehow unfavorably complicated the formulation.

Disclosed herein is the in vivo organ-selectivity of LNPs can be precisely tuned by simply changing the linker structure in the lipidoid tails without complicating the LNPs formulation. By changing the linker from ester bond (referred to as O-series) to amide bond (referred to as N-series), the mRNA delivery specificity enabled by LNPs was switched from the liver to the lung. A proteomic study was performed to try to explain why such a small structural difference between the O-series and N-series LNPs leads to dramatically different organ selectivity. These two tail structures may have a specific affinity to some distinct serum proteins that would somehow regulate the protein compositions of coronas formed on the surface of the LNPs during the circulation. It has been reported that the protein corona plays a key role in determining the in vivo fate of nanoparticles. Disclosed results reveale that the number and type of absorbed plasma proteins in the protein coronas of 306-O12B and 306-N16 LNPs are different. 14 distinct proteins were found among the top 20 most abundant proteins in these two LNPs. Even there are same sets of proteins are found in both protein coronas, they may have different biological functions because they are randomly oriented in the protein corona layer. One example is the ApoE, on one hand, it has been widely reported that endogenous ApoE could be absorbed to the LNPs surface following i.v. injection to facilitate the transport of encapsulated RNAs to hepatocytes by lipoprotein receptor-mediated endocytosis, while on the other hand, ApoE could also be deployed to direct nanoparticles to the brain. It was demonstrated that the binding model of 306-O12B and 306-N16B to albumin, the most abundant protein identified in the protein coronas of these two LNPs, was different that may subsequently influence the orientation as well as the biological function of albumin on the surface of LNPs.

Targeted RNA delivery to the lung is an attractive concept, which offers tremendous opportunities to target currently be defined as “undruggable” targets for the treatment of a broad spectrum of pulmonary diseases. Herein it is demonstrated that different pulmonary cells can be easily targeted by tuning the head structure of LNPs. Herein it is disclosed that 306-N16B LNP can specifically transport mRNA to the pulmonary endothelial cells, an important therapeutic target that involves acute respiratory distress syndrome, inflammation, and thrombosis. However, mRNA containing 113-N16B LNP was able to transfect not only pulmonary endothelial cells but also macrophages and even epithelial cells after systemic administration. Furthermore, it is also demonstrated that co-delivering Cas9 mRNA and sgRNA into one single 306-N16B LNP achieved genome editing in the lung of mice which may allow further development of genome editing based therapies for the treatment of pulmonary genetic diseases. More importantly, it is demonstrated that disclosed developed hybrid LNP was able to specifically deliver Tsc2 mRNA to TSC2-deficient TTJ tumor cells in a preclinical LAM model and significantly inhibit tumor cell growth, providing critical proof-of-concept for the use of LNP for the treatment of pulmonary diseases.

In summary, this study demonstrated a strong correlation of the structure-activity relationships between lipidoid tail chemistry and organ and cell selectivity for mRNA delivery. The mechanistic study using proteomics indicated that the protein composition in the protein coronas of these two LNPs are different, suggesting the unique tail linker structures may have a profound impact on the interaction of LNPs and biological fluids that substantially influence the targetability of LNPs. However, further studies should be performed to fully elucidate the effect of these unique proteins on organ targetability. The findings from this work provide a fundamental understanding of how lipid structure may affect the in vivo fate of LNPs by manipulating protein corona functions.

### INCORPORATION BY REFERENCE

All U.S. and PCT patent publications and U.S. patents mentioned herein are hereby incorporated by reference in their entirety as if each individual patent publication or patent was specifically and individually indicated to be incorporated by reference. In case of conflict, the present application, including any definitions herein, will control.

### OTHER EMBODIMENTS

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

1. A composition comprising: a pharmaceutical agent assembled with a lipid composition, wherein the lipid composition comprises a lipidoid having structural Formula (I): 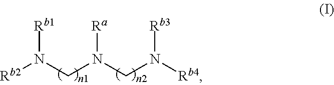
or a pharmaceutically acceptable salt thereof, wherein:
R^{a} is an alkyl;
n1 and n2 are each independently 1, 2, 3, or 4; and
R^{b1}, R^{b2}, R^{b3} and R^{b4} are each independently H, 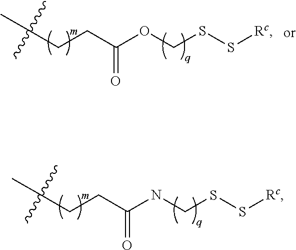
wherein:
* indicates the point of attachment to N;
each m is independently 1, 2, 3, 4, or 5;
each q is independently 1, 2, 3, 4, or 5; and
each R^{c} is independently an alkyl or an alkenyl; and
wherein at least one of R^{b1}, R^{b2}, R^{b3} and R^{b4} is not H.

2. The composition of claim 1, wherein R^{a} is C₁-C₃ alkyl.

3. The composition of claim 1, wherein n1 is 1, 2 or 3.

4. (canceled)

5. The composition of claim 1, wherein n2 is 1, 2 or 3.

6-9. (canceled)

10. The composition of claim 1, wherein R^{b1}, R^{b2}, R^{b3} and R^{b4} are each independently

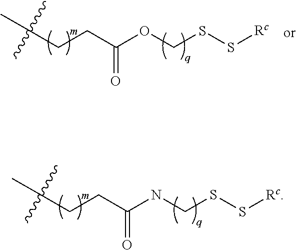

11. (canceled)

12. (canceled)

13. The composition of claim 1, wherein each R^{c} is independently C₄-C₁₆ alkyl or C₄₋₁₆ alkenyl.

14. The composition of claim 1, wherein each R^{c} is independently C₄-C₂₀ alkyl.

15. The composition of claim 1, wherein each m is independently 1, 2, or 3.

16. (canceled)

17. (canceled)

18. The composition of claim 1, wherein each q is independently 1, 2, 3, or 4.

19. (canceled)

20. (canceled)

21. The composition of claim 1, wherein the lipidoid of Formula (I), or the pharmaceutically acceptable salt thereof, is present in the lipid composition at a molar percentage of no more than about 60%.

22. The composition of claim 1, wherein the lipid composition further comprises a steroid.

23. (canceled)

24. The composition of claim 1, wherein the lipid composition further comprises a polymer-conjugated lipid.

25. (canceled)

26. The composition of claim 1, wherein the lipid composition further comprises a phospholipid.

27. (canceled)

28. (canceled)

29. The composition of claim 1, wherein the pharmaceutical agent comprises a polynucleotide, an oligonucleotide, a polypeptide, an oligopeptide, a small molecule compound, or any combination thereof regulate a target gene or a gene product thereof.

30-40. (canceled)

41. The composition according to claim 1, wherein, in the lipidoid of Formula (I) or the pharmaceutically acceptable salt thereof,
R^{b1}, R^{b2}, R^{b3} and R^{b4} are each independently H or 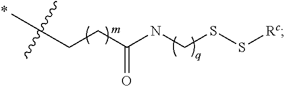
and
at least one of R^{b1}, R^{b2}, R^{b3} and R^{b4} is

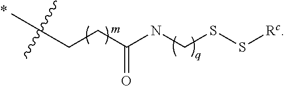

42-56. (canceled)

57. The composition of claim 0, wherein the lipidoid is selected from: 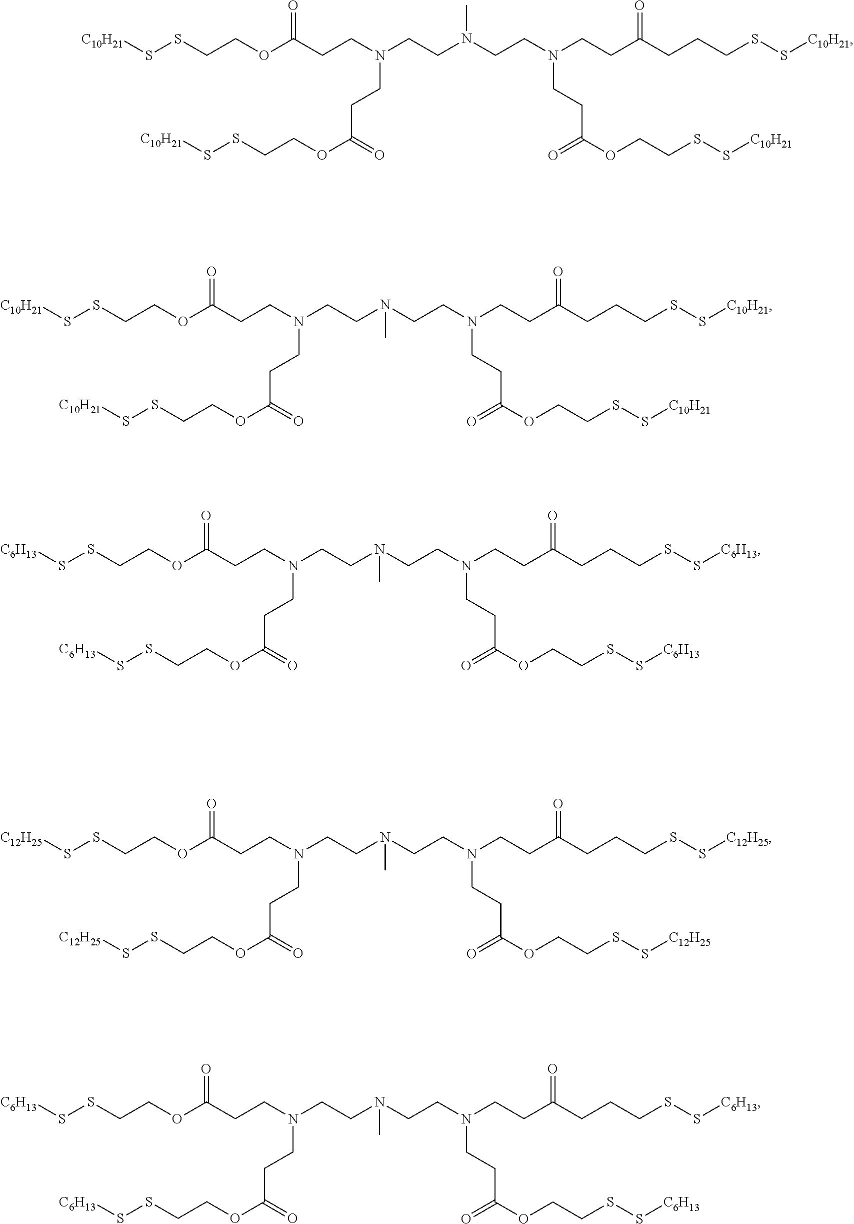
and any pharmaceutically acceptable salt of any of the foregoing.

58-64. (canceled)

65. A method for preferential delivery of a pharmaceutical agent to a lung or a lung cell in a subject in need thereof, the method comprising administering the composition according to claim 41, thereby providing a greater amount, expression or activity of the pharmaceutical agent in the lung or the lung cell of the subject as compared to that achieved in a non-lung organ or a non-lung cell in the subject.

66-68. (canceled)

69. The composition according to claim 1, wherein, in the lipidoid of Formula (I) or the pharmaceutically acceptable salt thereof,
R^{b1}, R^{b2}, R^{b3} and R^{b4} are each independently H or 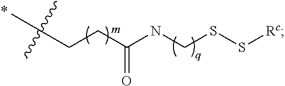
and
at least one of R^{b1}, R^{b2}, R^{b3} and R^{b4} is

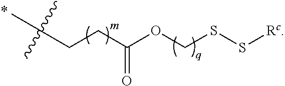

70-84. (canceled)

85. The composition of claim 0, wherein the lipidoid is selected from: 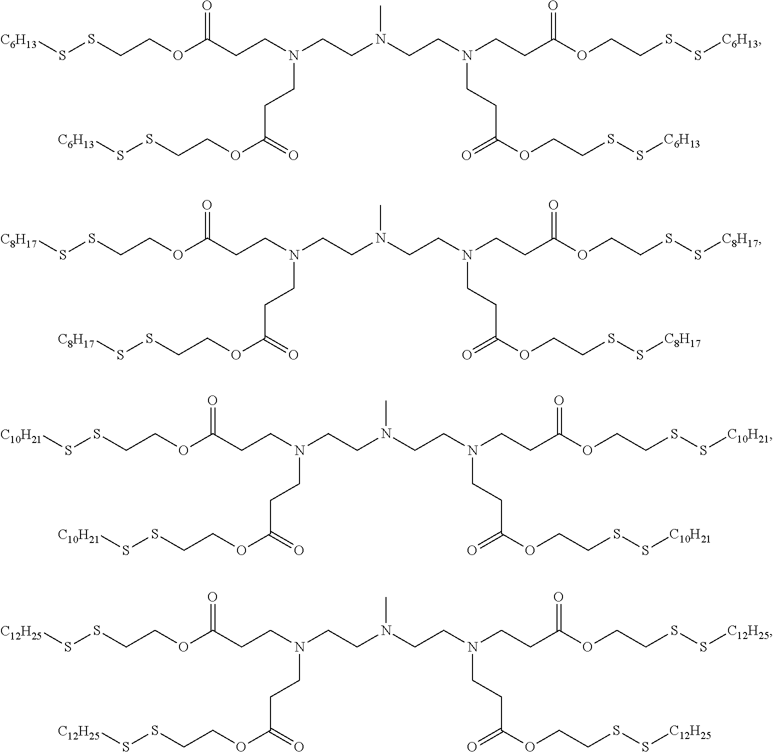 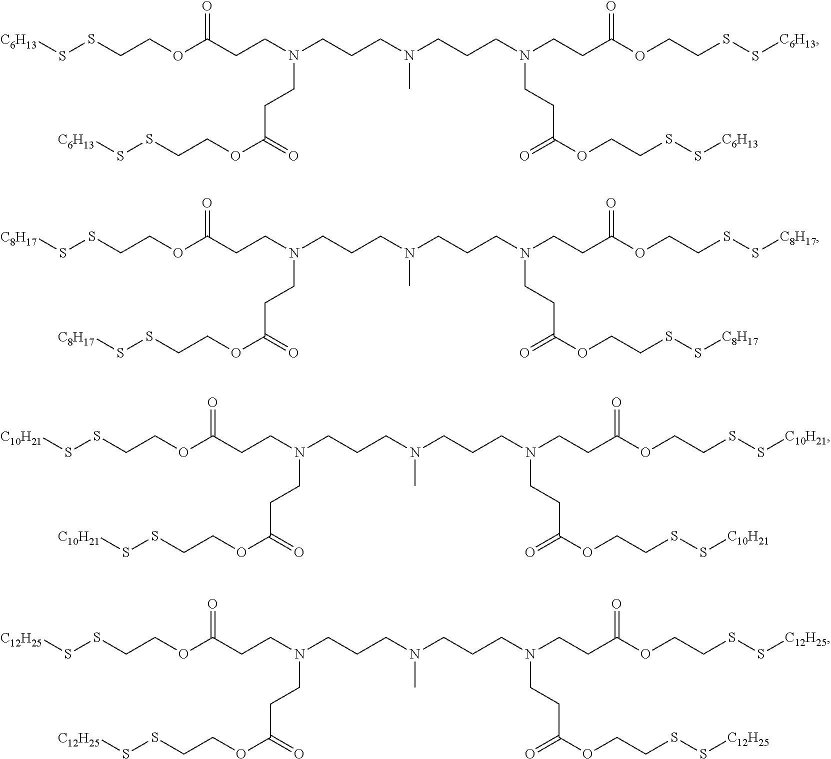
and any pharmaceutically acceptable salt of any of the foregoing.

86-92. (canceled)

93. A method for preferential delivery of a pharmaceutical agent to liver or a liver cell in a subject in need thereof, the method comprising administering the composition according to claim 69, thereby providing a greater amount, expression or activity of the pharmaceutical agent in the liver or the liver cell of the subject as compared to that achieved in a non-liver organ or a non-liver cell in the subject.

94-96. (canceled)
